(19) United States
(12) Patent Application Publication
(10) Pub. No.: US 2024/0158809 A1
(43) Pub. Date: May 16, 2024

(54) HIGH-TRANSDUCING HSV VECTORS

(71) Applicant: PeriphaGen, Inc., Pittsburgh, PA (US)

(72) Inventors: David M. Krisky, Pittsburgh, PA (US); James B. Wechuck, Pittsburgh, PA (US)

(73) Assignee: PeriphaGen, Inc., Pittsburgh, PA (US)

(21) Appl. No.: 18/359,013

(22) Filed: Jul. 26, 2023

Related U.S. Application Data
(62) Division of application No. 16/088,393, filed on Sep. 25, 2018, now Pat. No. 11,753,653.

(51) Int. Cl. C12N 15/86; A61K 48/00; C12N 7/00

(52) U.S. Cl. CPC C12N 15/86; A61K 48/0025; C12N 7/00; C12N 2710/16621; C12N 2710/16643; C12N 2710/16662; C12N 2830/008

(57) ABSTRACT

Disclosed herein are high transducing replication defective herpes simplex virus (HSV) vectors of McKrae strain.

### CROSS REFERENCE TO RELATED APPLICATIONS

The present application is a Division of U.S. application Ser. No. 16/088,393, filed Sep. 25, 2018, which is the National Stage of International application Ser. No. 17/24,092, filed Mar. 24, 2017, which claims priority to U.S. Provisional Application No. 62/313,391, filed Mar. 25, 2016, the entire contents of which are herein incorporated by reference.

### SEQUENCE LISTING

The contents of the electronic sequence listing (P113870000US02-SEQ-EMB.xml; Size: 180,145 bytes; and Date of Creation: Jul. 21, 2023) is herein incorporated by reference in its entirety.

### BACKGROUND

Systemic delivery of certain therapeutic agents can be problematic for agents with poor pharmacokinetics and/or a risk of off target adverse effects. Local injection at particular target sites may require highly invasive techniques or be infeasible. Delivery of agents by viral vectors allows the ability to specifically target cell populations to provide local production and/or delivery of agents.

### SUMMARY OF THE INVENTION

The present disclosure provides compositions and methods for viral vector delivery of agents to target cells.

In some embodiments, the disclosure provides variants of herpes simplex virus

(HSV) McKrae strain whose genome contains an alteration such that the variant fails to express a functional protein characterized by an amino acid sequence of SEQ ID NO: 16.

In some embodiments, the disclosure provides variants of herpes simplex virus McKrae strain having a truncated genome of total size less than about 150,000 base pairs and including a deletion of one or more residues within an element corresponding to residues 126049 to 130014 of SEQ ID NO: 1.

In some embodiments, the disclosure provides vectors comprising a variant herpes simplex virus (HSV) McKrae strain genome which genome contains an alteration such that the variant fails to express a functional protein characterized by an amino acid sequence of SEQ ID NO: 2. In some embodiments, the vector comprises a neuron specific promoter. In some embodiments, the promoter is a calcitonin gene-related peptide (CGRP) promoter.

In some embodiments, the vector comprises a human cytomegalovirus (HCMV) enhancer. In some embodiments, the vector comprises a bovine growth hormone (BGH) polyadenylation signal. In some embodiments, the vectors comprise a nucleic acid that encodes a therapeutic polypeptide.

In some embodiments, the disclosure provides cells transduced with a HSV McKrae strain viral vector as described herein.

In some embodiments, the disclosure provides pharmaceutical compositions comprising an HSV McKrae strain viral vector as described herein and a pharmaceutically acceptable carrier.

In some embodiments, the disclosure provides methods of propagating a vector comprising a variant herpes simplex virus (HSV) McKrae strain genome which genome contains an alteration such that the variant fails to express a functional protein characterized by an amino acid sequence of SEQ ID NO: 16, the method comprising steps of: (i) infecting cultured ICP4 complementing cells containing DNA encoding HSV protein ICP4 with the vector, and (ii) isolating supernatant from the culture of step (i).

In some embodiments, the method comprises a step of purifying vector in the supernatant by chromatography. In some embodiments, the method comprises a step of concentrating the purified vector. In some embodiments, purified vector is concentrated by tangential flow filtration.

In some embodiments, the disclosure provides methods of preparing a vector comprising a variant herpes simplex virus (HSV) McKrae strain genome which genome contains an alteration such that the variant fails to express a functional protein characterized by an amino acid sequence of SEQ ID NO: 16, and wherein the vector expresses a marker element, the method comprising incubating cells transfected with:
(a) a first nucleic acid molecule:
   (i) comprising a portion of HSV McKrae strain genome but does not encode a functional protein characterized by an amino acid sequence of SEQ ID NO: 16; and
   (ii) comprising a first homology region (HR1) and a second homology region (HR2), and
(b) a second nucleic acid molecule comprising a sequence that encodes a marker element, wherein the sequence is flanked by a first homology region (HR1′) and a second homology region (HR2′), wherein HR1 is homologous to HR1′ and HR2 is homologous to HR2′ such that the sequence that encodes the marker element in the second nucleic acid molecule integrates into the first nucleic acid molecule via homologous recombination.

In some embodiments, the cells are ICP4 complementing cells. In some embodiments, the cells complement ICP4 and at least one other viral gene. In some embodiments, the cells complement ICP4 and at least one immediate early gene. In some embodiments, the cells are ICP4, ICP27, and UL55 complementing cells. In some embodiments, the cells are ICP4, ICP22, and ICP47 complementing cells.

In some embodiments, the marker element is a polypeptide. In some embodiments, the polypeptide is detectable by fluorescence. In some embodiments, the marker element is a green fluorescent peptide. In some embodiments, the method comprises a step of purifying viral plaques that express the marker element.

In some embodiments, the disclosure provides methods of preparing a vector comprising a variant herpes simplex virus (HSV) McKrae strain genome which genome contains an alteration such that the variant fails to express a functional protein characterized by an amino acid sequence of SEQ ID NO. 16, and wherein the vector expresses an agent of interest, the method comprising incubating cells transfected with:
a) a first nucleic acid molecule:
   (i) comprising a portion of HSV McKrae strain genome but does not encode a functional protein characterized by an amino acid sequence of SEQ ID NO: 16; and
   (ii) comprising a sequence that encodes a marker element, wherein the sequence that encodes the marker element is flanked by a first homology region (HR1) and a second homology region (HR2); and
(b) a second nucleic acid molecule comprising a sequence that encodes an agent of interest, wherein the sequence encoding the agent of interest is flanked by a first homology region (HR1′) and a second homology region (HR2′), wherein HR1 is homologous to HR1′ and HR2 is homologous to HR2′ such the sequence encoding the agent of interest is integrated into the first nucleic acid molecule via homologous recombination.

In some embodiments, the cells are ICP4 complementing cells. In some embodiments, the cells complement ICP4 and at least one other viral gene. In some embodiments, the cells complement ICP4 and at least one immediate early gene. In some embodiments, the cells are ICP4, ICP27, and UL55 complementing cells. In some embodiments, the cells are ICP4, ICP22, and ICP47 complementing cells.

In some embodiments the method comprises a step of purifying viral plaques that do not express the marker element.

In some embodiments, the disclosure provides methods of expressing a polypeptide in dorsal root ganglion (DRG) of a subject comprising administering to the subject an HSV McKrae strain vector as described herein. In some embodiments, the vector is administered in vivo. In some embodiments, the vector is administered by contact with skin. In some embodiments, the vector is administered by intradermal injection.

In some embodiments, the disclosure provides methods of measuring transduction efficiency in dorsal root ganglion (DRG) of an HSV McKrae strain viral vector comprising (a) contacting the skin of an animal with an HSV McKrae strain viral vector (b) removing DRG tissue from the animal, and (c) assaying the number of HSV genomes transduced in the DRG. In some embodiments, the number of genomes is measured by an amplification technique. In some embodiments, the number of genomes is measured by quantitative polymerase chain reaction (PCR).

In some embodiments, the disclosure provides methods of measuring transduction efficiency in dorsal root ganglion (DRG) of an HSV McKrae strain viral vector that contains an expression cassette comprising a polypeptide payload, the method comprising steps of: (a) contacting the skin of an animal with an HSV McKrae strain viral vector, (b) removing DRG tissue from the animal, and (c) assaying the amount of a polypeptide encoded by a nucleic acid of the expression cassette. In some embodiments, the amount of polypeptide is measured by an immunoassay. In some embodiments, the amount of polypeptide is measured by an enzyme linked immunosorbent assay (ELISA).

### BRIEF DESCRIPTION OF THE DRAWINGS

The drawings are for illustration purposes only, not for limitation.

FIG. 1 depicts an exemplary graph that shows the number of HSV genomes per L4-L6 dorsal root ganglia (DRG) detected in a qPCR assay as a result of different doses of replication-defective viral vector injected into the footpad.

FIG. 2 depicts an exemplary graph that shows the total number of transcripts of payload in L4-L6 DRG at 5, 14, 47, 77, and 131 days post administration with HSV viral vectors having a HCMV promoter.

FIG. 3 depicts an exemplary graph that shows the number of transcripts of payload per genome in L4-L6 DRG at 5, 14, 47, 77, and 131 days post administration with HSV viral vectors having a HCMV promoter.

FIG. 4 depicts an exemplary graph that shows the number of GFP transcripts per genome and the number of HSV-1 genomes per L4-L6 DRG as a result of administering viral vectors with different promoters.

FIG. 5 depicts an exemplary graph that shows the total number of GFP transcripts and total transcripts per genome in L4-L6 DRG over 18 days after administration of HSV viral vectors with tissue specific promoters.

FIG. 6 depicts an exemplary graph that shows the number of total GFP transcripts in L4-L6 DRG over 8 weeks after administration of HSV viral vectors with tissue specific promoters.

FIG. 7 depicts an exemplary graph that shows the total number of GFP transcripts in L4-L6 DRG over 8 weeks after administration of HSV viral vectors with tissue specific promoters.

FIG. 8 depicts an exemplary graph that shows the total number of transcripts of payload per genome in L4-L6 DRG after administration with HSV viral vectors having a human cytomegalovirus (HCMV) promoter compared to HSV viral vectors having a chimeric calcitonin gene-related peptide (CGRP) promoter with an HCMV enhancer.

FIG. 9 depicts an exemplary HSV McKrae strain nucleotide sequence (SEQ ID NO: 1) which is identified as accession number JQ730035.1

FIG. 10 depicts an exemplary HSV McKrae strain ICP4 amino acid sequence (SEQ ID NO: 2).

FIG. 11 depicts an exemplary HSV McKrae strain ICP22 amino acid sequence (SEQ ID NO: 3).

FIG. 12 depicts an exemplary HSV McKrae strain ICP47 amino acid sequence (SEQ ID NO: 4).

FIG. 13 depicts an exemplary HSV McKrae strain nucleotide sequence of ICP4 (SEQ ID NO: 5).

FIG. 14 depicts an exemplary HSV McKrae strain nucleotide sequence of ICP22 (SEQ ID NO: 6).

FIG. 15 depicts an exemplary HSV McKrae strain nucleotide sequence ICP47 (SEQ ID NO: 7).

FIG. 16 depicts an exemplary human cytomegalovirus enhancer nucleotide sequence (SEQ ID NO: 8).

FIG. 17 depicts an exemplary calcitonin gene-related peptide nucleotide sequence (SEQ ID NO: 9).

FIG. 18 depicts an exemplary bovine growth hormone polyadenylation signal (SEQ ID NO: 10).

FIG. 19 depicts the extent of the ICP4 deletion resulting in the removal of the upstream promoter sequences of two additional immediate early viral genes: ICP22 and ICP47.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time.

Agent: As used herein, the term “agent” refers to a compound or entity of any chemical class including, for example, polypeptides, nucleic acids, saccharides, lipids, small molecules, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc.

Amelioration: As used herein, the term “amelioration” refers to the prevention, reduction or palliation of a state, or improvement of the state of a subject. Amelioration includes, but does not require complete recovery or complete prevention of a disease, disorder or condition.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%6, or less in either direction (greater than or less than) of the stated reference value unless otherwise stated or otherwise evident from the context (except where such number would exceed 100% f a possible value).

Characteristic sequence: As used herein, the term “characteristic sequence” refers to a sequence that is found in all members of a family of polypeptides or nucleic acids, and therefore can be used by those of ordinary skill in the art to define members of the family.

Combination therapy: As used herein, the term “combination therapy” refers to those situations in which a subject is simultaneously exposed to two or more therapeutic regimens (e.g., two or more therapeutic agents). In some embodiments, two or more agents or may be administered simultaneously; in some embodiments, such agents may be administered sequentially; in some embodiments, such agents are administered in overlapping dosing regimens.

Composition: As used herein, the term “composition” or a “pharmaceutical composition” refers to the combination of two or more agents as described herein for co-administration or administration as part of the same regimen. It is not required in all embodiments that the combination of agents result in physical admixture, that is, administration as separate co-agents each of the components of the composition is possible; however many patients or practitioners in the field may find it advantageous to prepare a composition that is an admixture of two or more of the ingredients in a pharmaceutically acceptable carrier, diluent, or excipient, making it possible to administer the component ingredients of the combination at the same time.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present disclosure, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% f the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker element: As used herein, the term “marker element” refers to a detectable or selectable agent. In some embodiments, a “marker element” is a detectable or selectable nucleic acid sequence. In some embodiments a “marker element” is an expression product (e.g., RNA or protein) whose presence or absence is detectable and/or selectable in cells. In some embodiments, an expression product is or comprises an enzyme. In some embodiments, an expression product is a fluorophore.

Nucleic acid: As used herein, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, CS-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof) In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the patient is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation, topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include—sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevent or prevention: As used herein, the terms “prevent” or “prevention”, when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Subject: As used herein, the term “subject” refers to a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Treatment: As used herein, the term “treatment” (also “treat” or “treating”) refers to any administration of a substance that partially or completely alleviates, ameliorates, relieves, inhibits, delays onset of, reduces severity of, and/or reduces incidence of one or more symptoms, features, and/or causes of a particular disease, disorder, and/or condition (e.g., neuropathy). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated to a viral genome or portion thereof. Certain vectors are capable of autonomous replication in a host cell into which they are introduced (e.g., bacterial vectors having a bacterial origin of replication, episomal mammalian vectors, herpes simplex virus (HSV) vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION

Various aspects of the invention are described in detail in the following sections. The use of sections is not meant to limit the invention. Each section can apply to any aspect of the invention. In this application, the use of “or” means “and/or” unless stated otherwise or clear from context to be disjunctive.

The present disclosure provides, among other things, compositions comprising HSV vectors and methods for use and production of same. In particular, the present disclosure relates to McKrae strain vectors for the delivery of payloads to neuronal cells.

### Viral Vectors and HSV

Viral vectors can be used to facilitate the transfer of nucleic acids into cells. Known viral vectors include those derived from retroviruses, adenoviruses, adeno-associated virus (AAV), vaccinia virus, and baculovirus. Vectors derived from herpes simplex viruses (HSV), such as herpes simplex virus 1 (HSV-1) and herpes simplex virus-2 (HSV-2) are particularly useful for delivery of agents to specifically targeted tissues. Considerations for choosing a particular vector and delivery system include, for example, characteristics of target cells, desired longevity of expression, virulence and invasiveness of the vector; and, size of the genetic material to be transferred.

HSV-1 vectors can typically accommodate up to 25 kb of foreign DNA sequences. HSV-1 has an approximate 152-kb double-stranded linear DNA genome that can be maintained episomally in the nucleus of cells. The HSV-1 virion is enveloped and approximately 110 nm in diameter. Viral infection is initiated in epithelial cells of the skin or mucosal membranes by binding of the viral envelope glycoproteins to heparin sulfate moieties on the plasma membrane. HSV is particularly well suited for the delivery of genes to the nervous system and possesses a natural tropism for sensory neurons. The virus can establish a latent state in which viral genomes persist for the life of the host as an intranuclear episomal element. The life-long persistence of latent genomes in human trigeminal ganglia without the development of sensory loss or histologic damage to the ganglia exemplifies the effectiveness of the latency mechanisms. Wild-type HSV virus may be reactivated from latency under the influence of a variety of stresses. However, recombinant viral vectors that are rendered replication defective retain the ability to establish a persistent quiescent state in neurons yet are unable to replicate (or reactivate) in the nervous system.

Vectors based upon HSV-1 may have one or more HSV genes necessary for replication rendered nonfunctional (e.g., by deletion or disruption). HSV genes necessary for replication include, for example, immediate early genes such as ICP4 and ICP 27. In some embodiments, the disclosure provides replication defective HSV vectors with one or more of ICP9, ICP4, ICP22, ICP27, and ICP47 deleted or disrupted. In some embodiments, the disclosure provides HSV vectors with a nonfunctional ICP4 gene. In some embodiments, the disclosure provides HSV vectors with nonfunctional ICP4, ICP22, and ICP47 genes. In some embodiments, the disclosure provides an HSV vector with ICP4 deleted and ICP22 and ICP47 disrupted. In some embodiments, the disclosure provides an HSV vector with ICP4 deleted and expression of ICP22 and ICP47 disrupted or delayed. In some embodiments, the disclosure provides an HSV vector with ICP4 deleted ICP0, ICP22, ICP27, and/or ICP47 not expressed as immediate early genes.

HSV-1 vectors that have deleted HSV genes can be produced in cell lines that express the deficient protein in trans. In some embodiments, HSV-1 vectors are produced in a mammalian cell line. In some embodiments, HSV-1 vectors are produced in a mammalian cell line of Vero lineage. In some embodiments, the cell line expresses ICP4. In some embodiments, the cell line expresses one or more of ICP0, ICP4, ICP22, ICP27, and ICP47. In some embodiments, the cell line expresses ICP4 and at least one additional immediate early gene. In some embodiments, the cell line expresses ICP4, ICP22, and ICP 47. In some embodiments, the cell line expresses ICP4, ICP22, and UL55. In some embodiments, the cell line expresses ICP4, ICP27 and UL55. In some embodiments, the cell line comprises a nucleic acid molecule having a simian virus 40 polyadenylation signal (SV40 pA) In some embodiments, viral vectors are produced in Vero 6-5C cells. In some embodiments, viral vectors are produced in Vero D cells.

### McKrae Strain

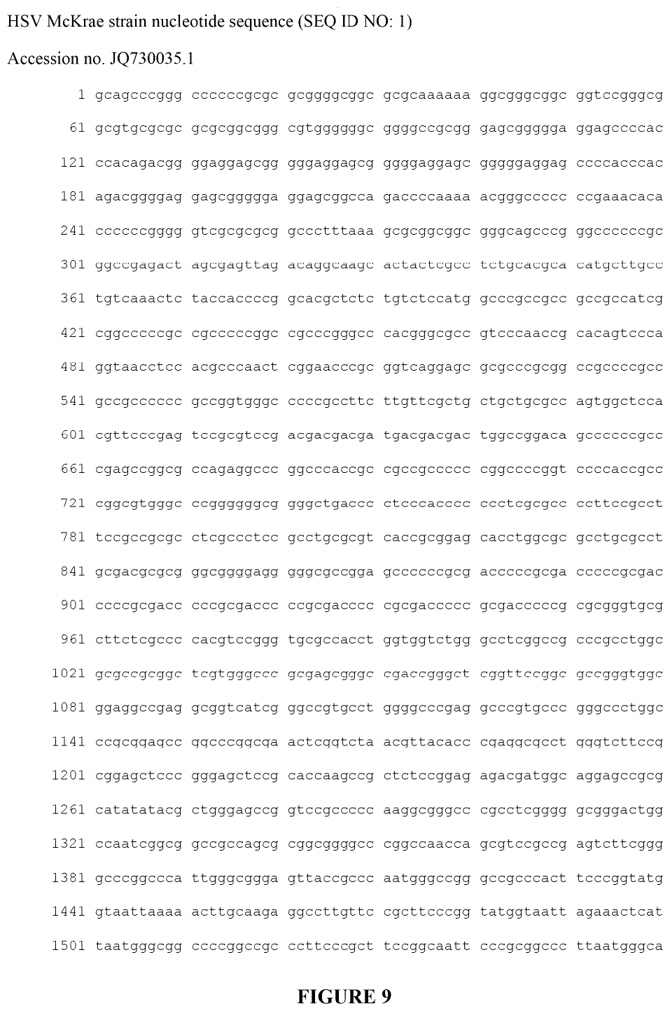

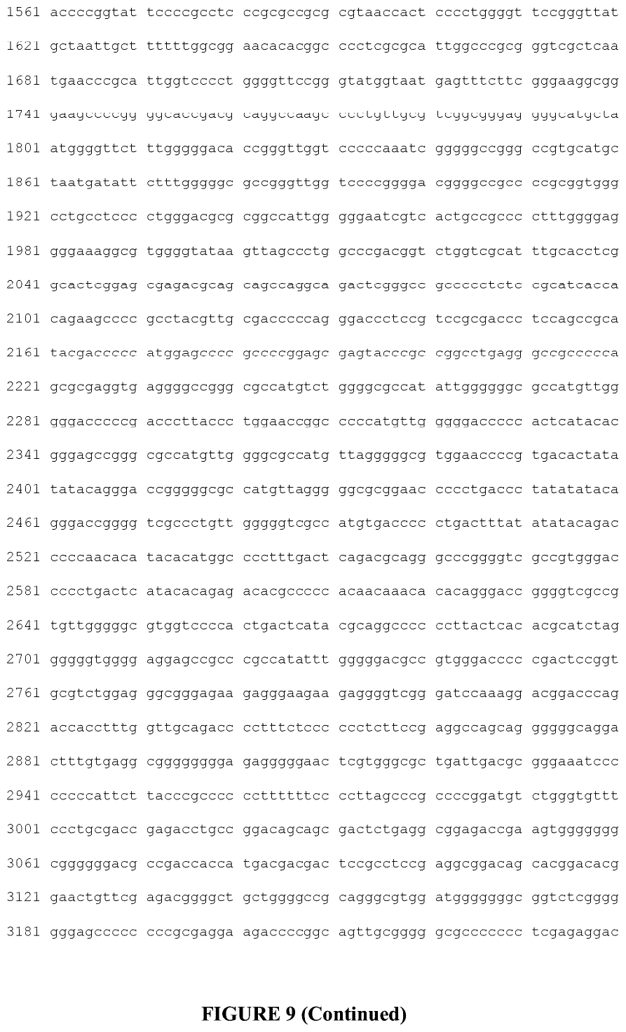

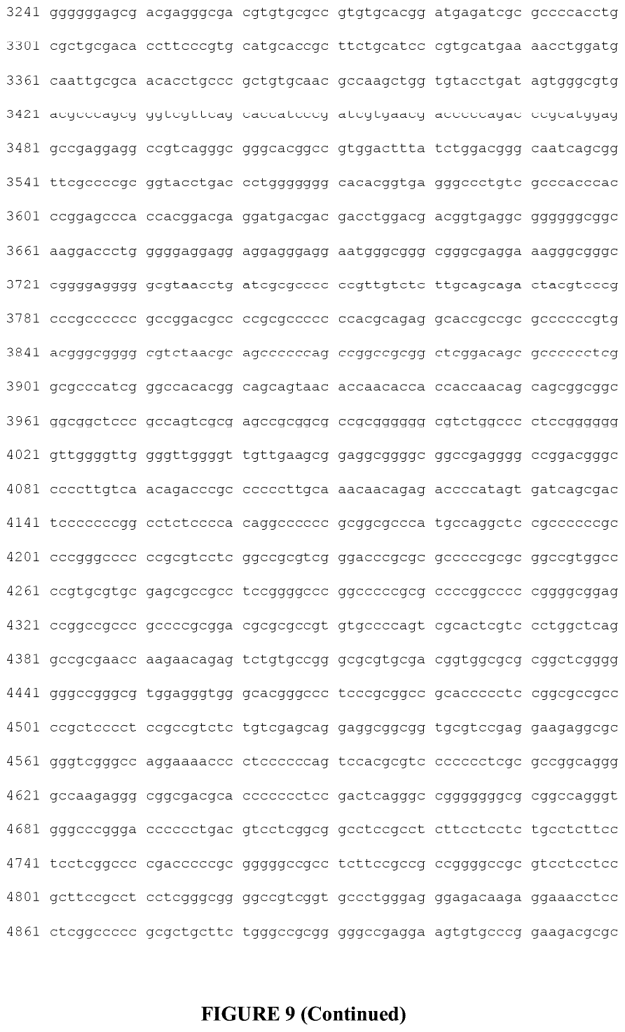

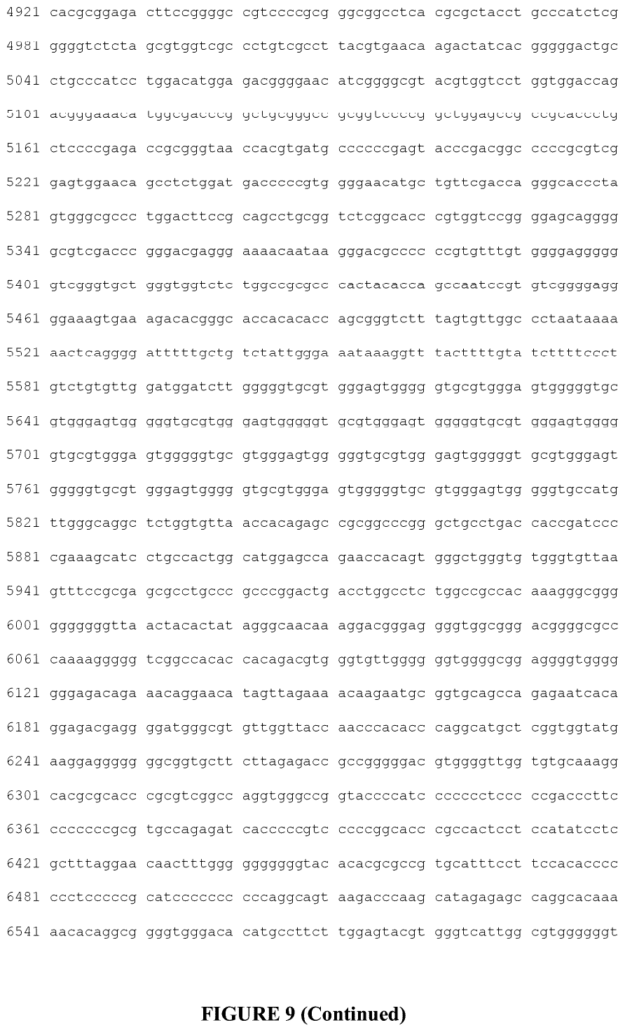

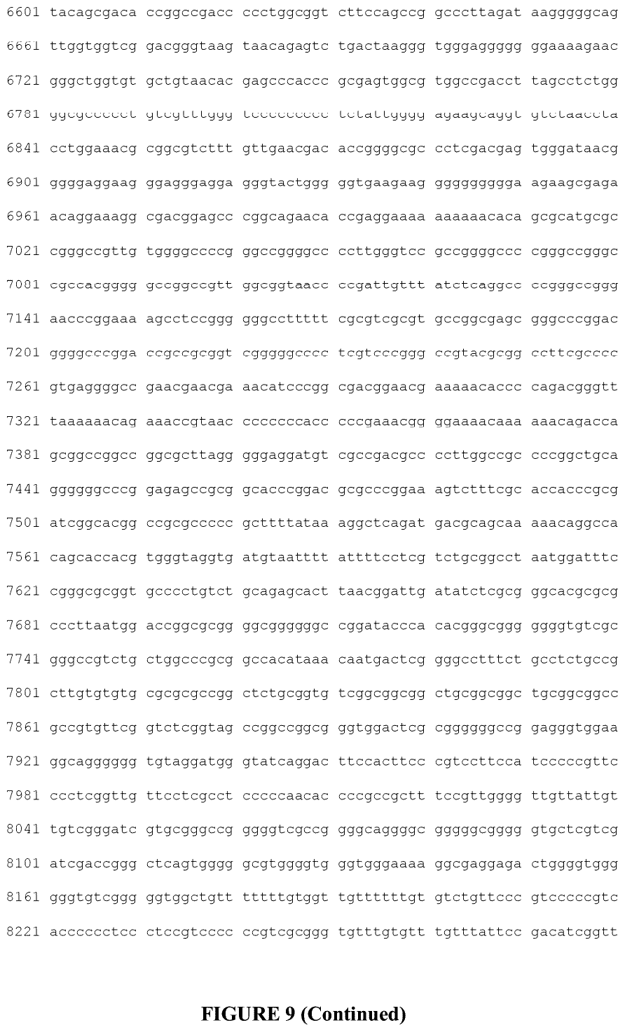

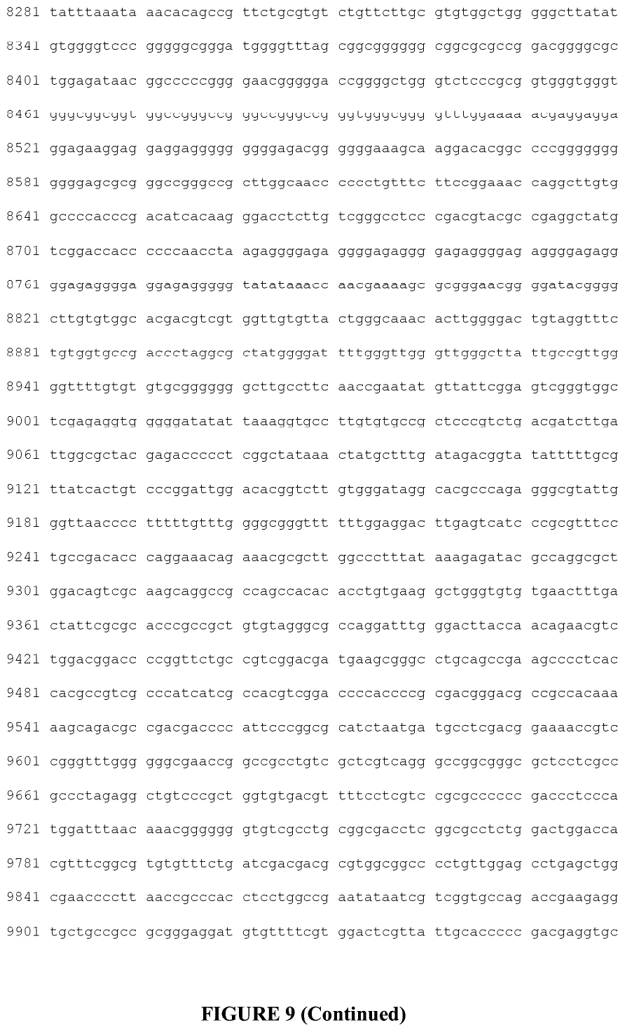

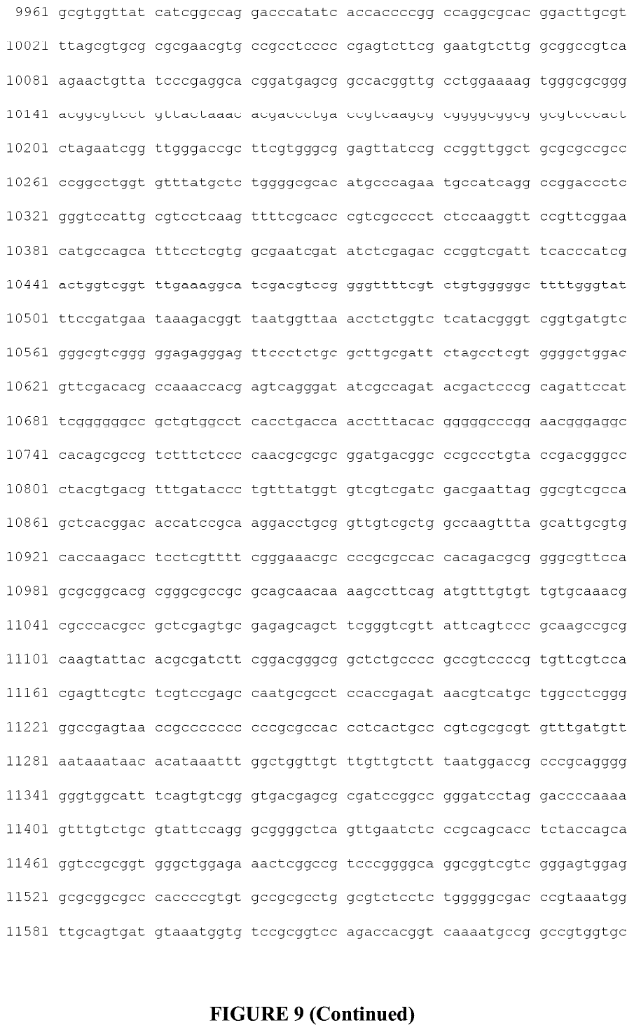

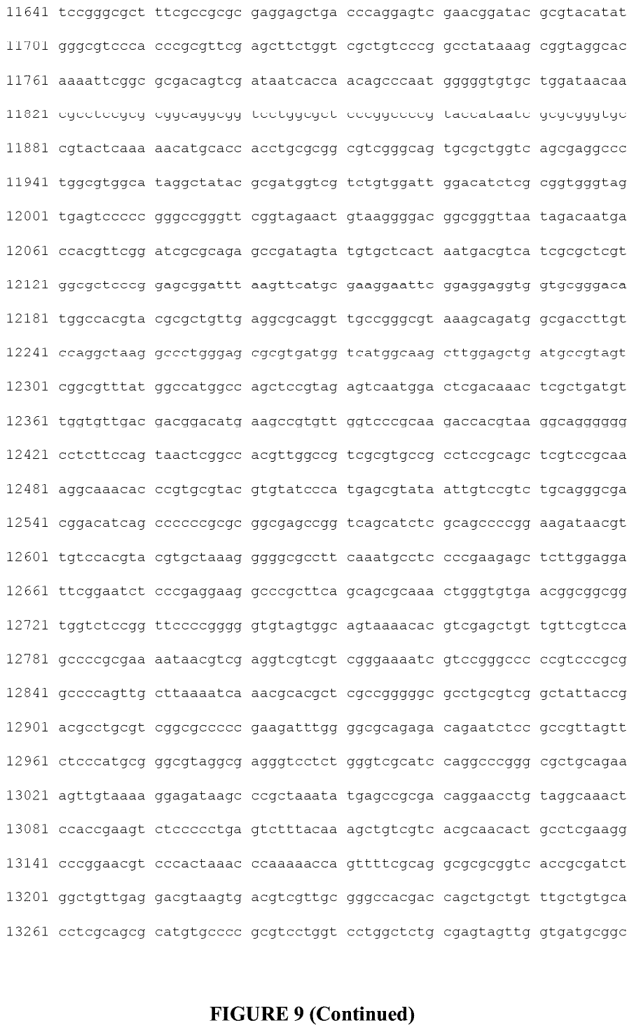

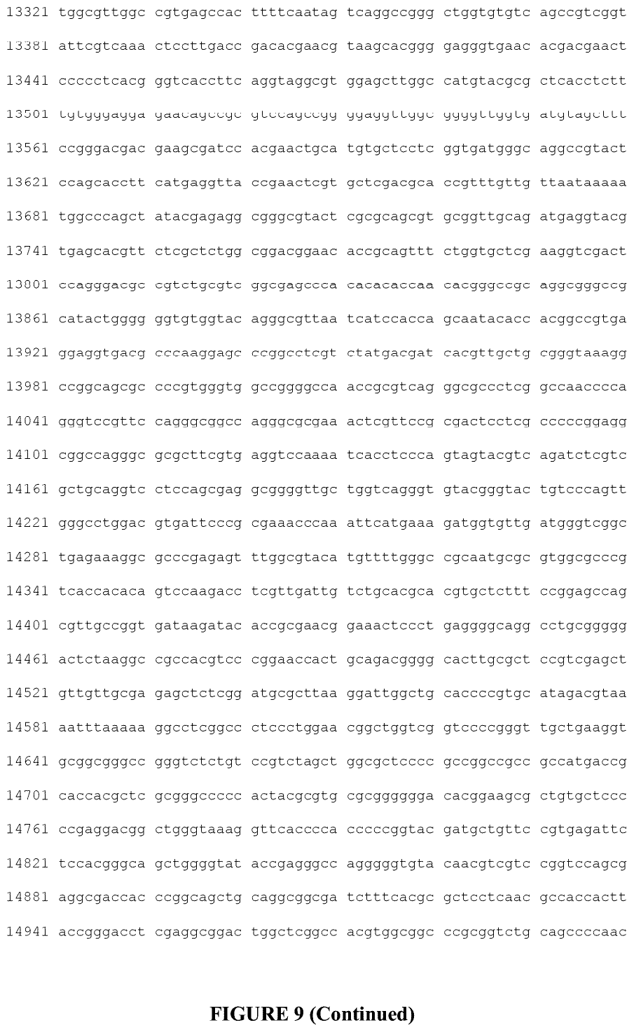

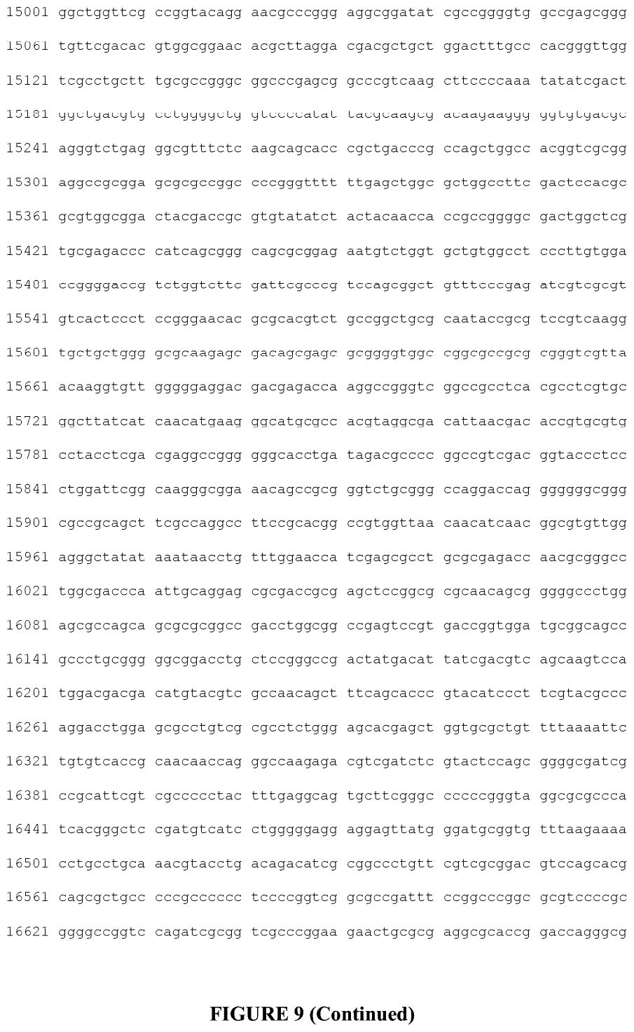

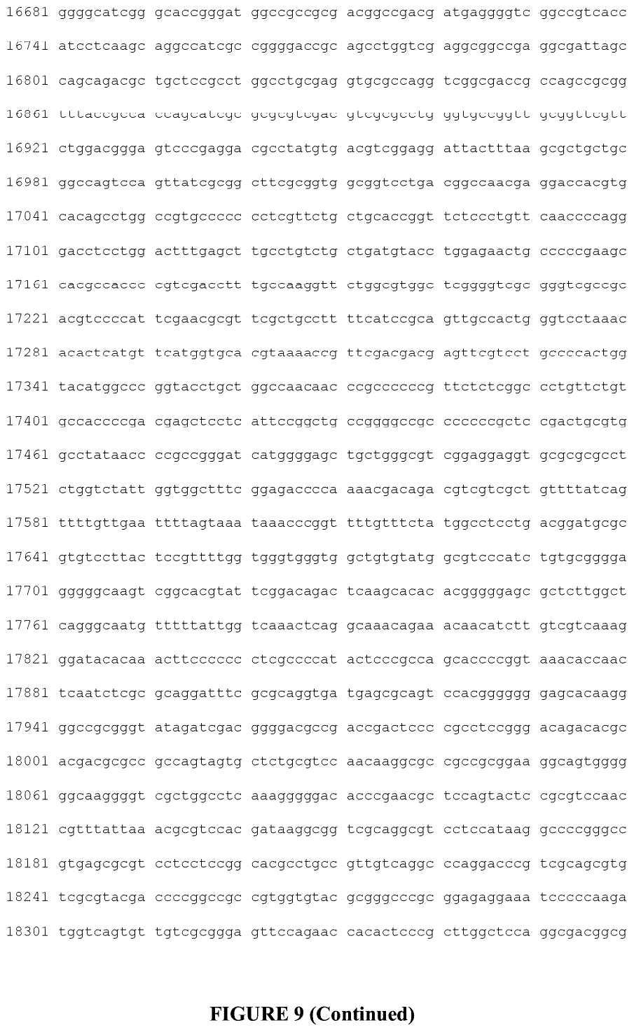

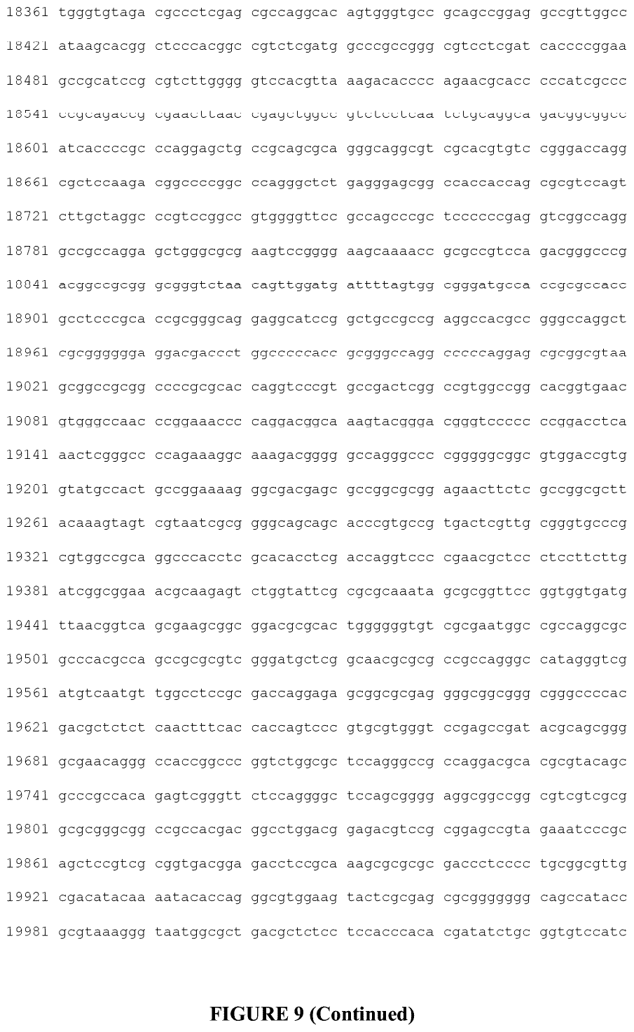

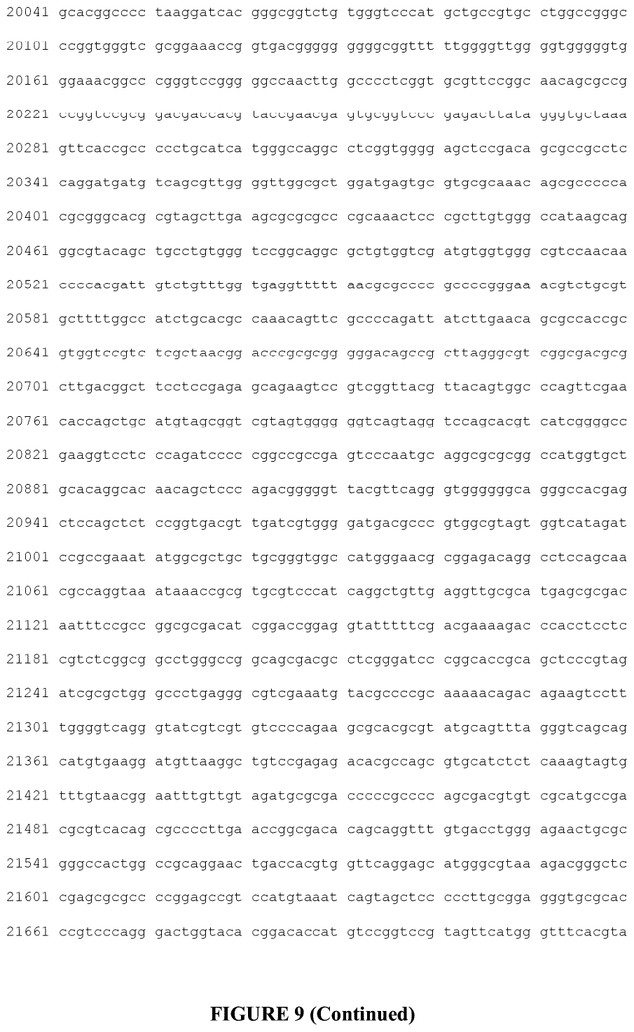

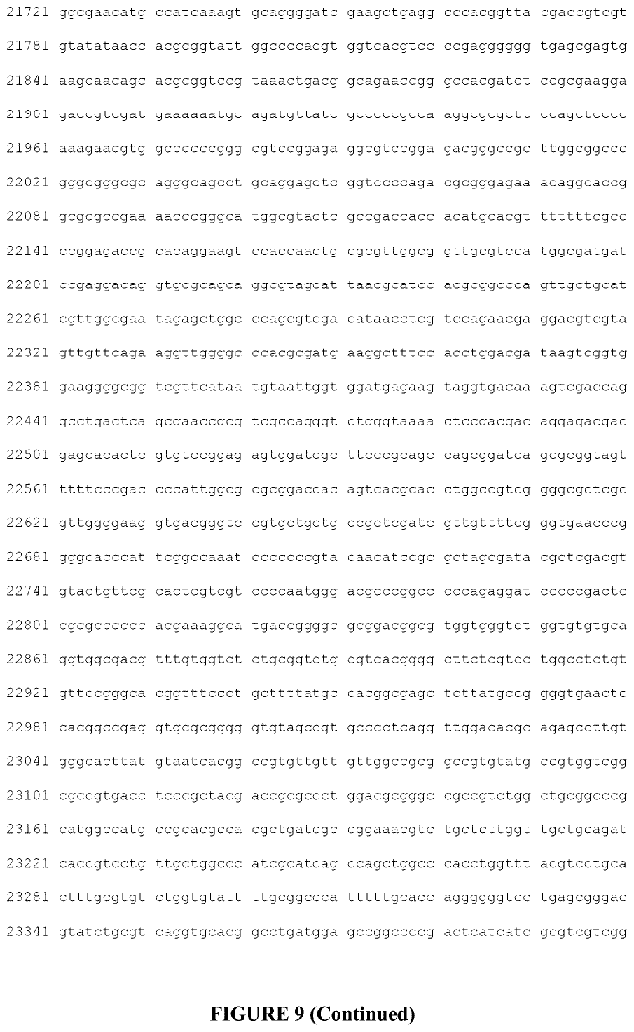

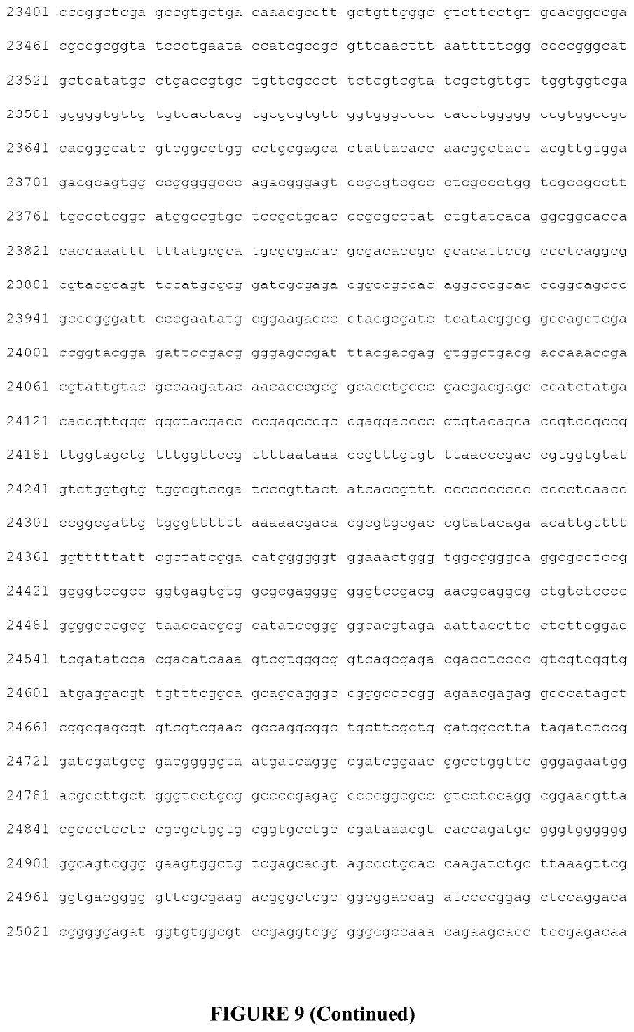

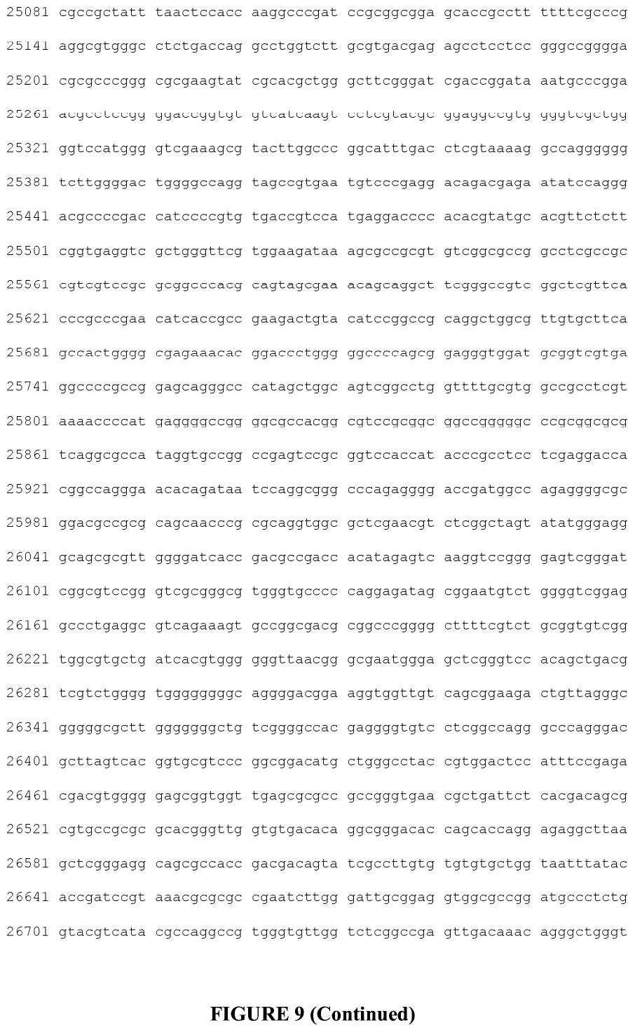

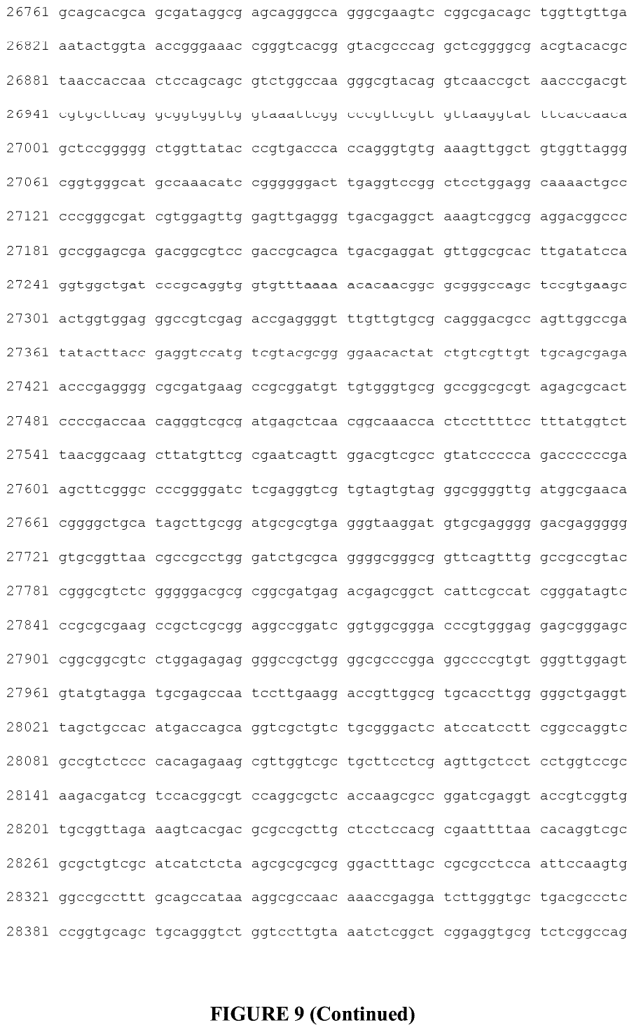

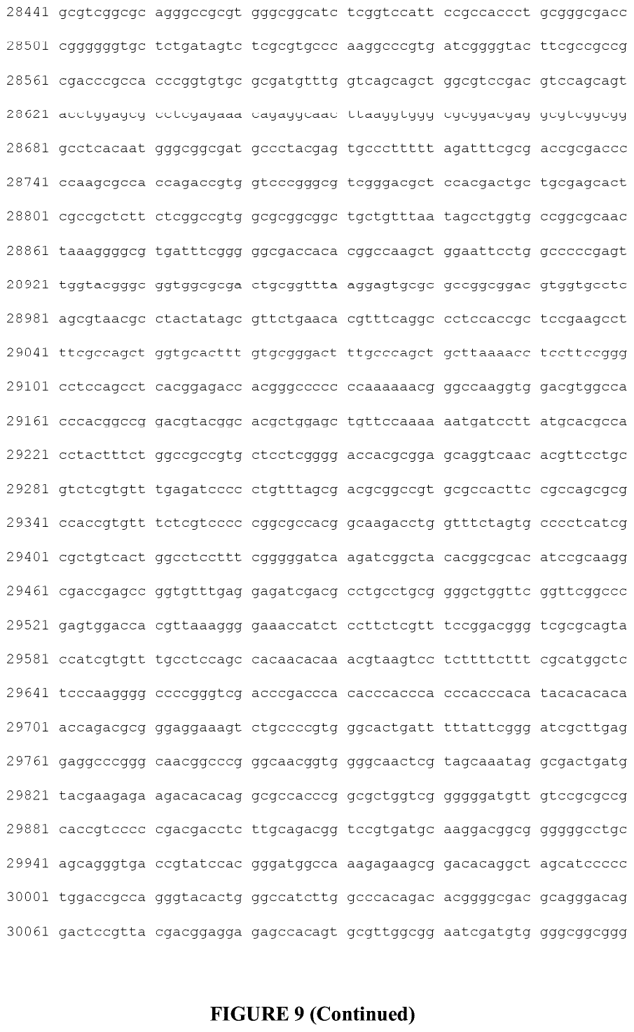

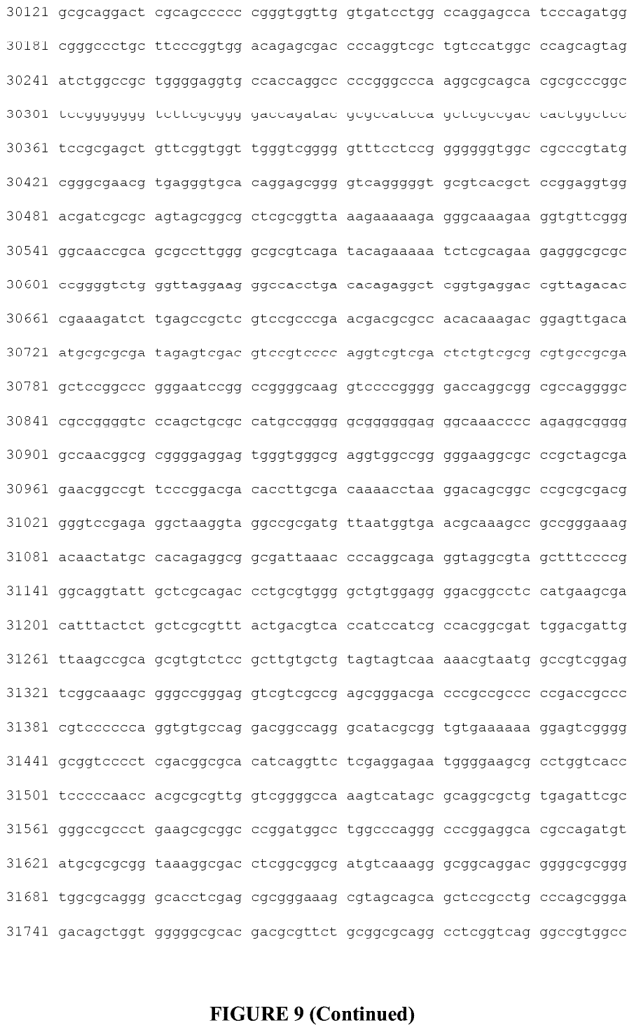

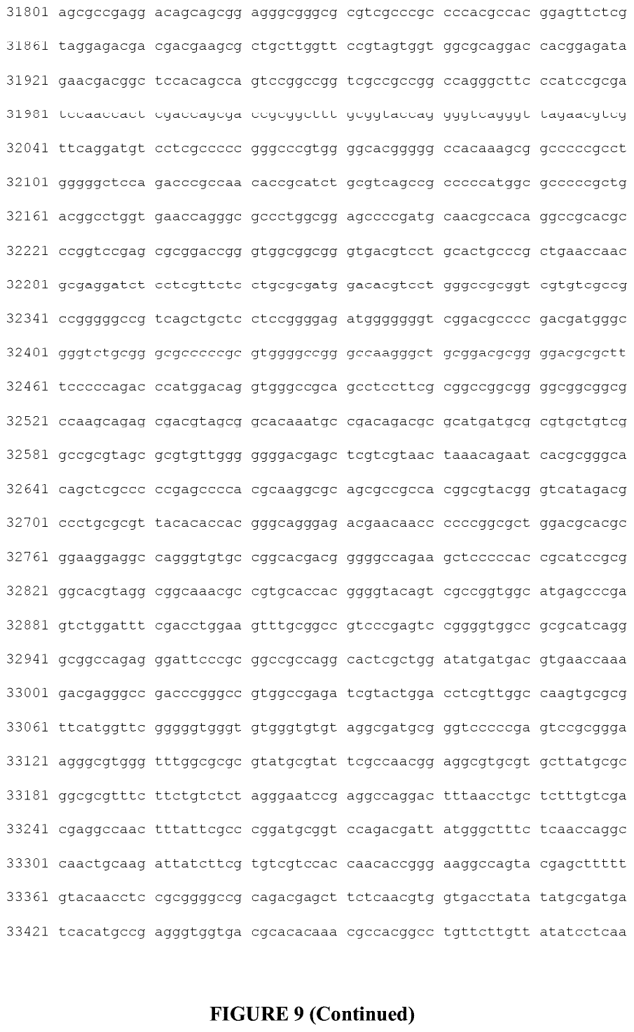

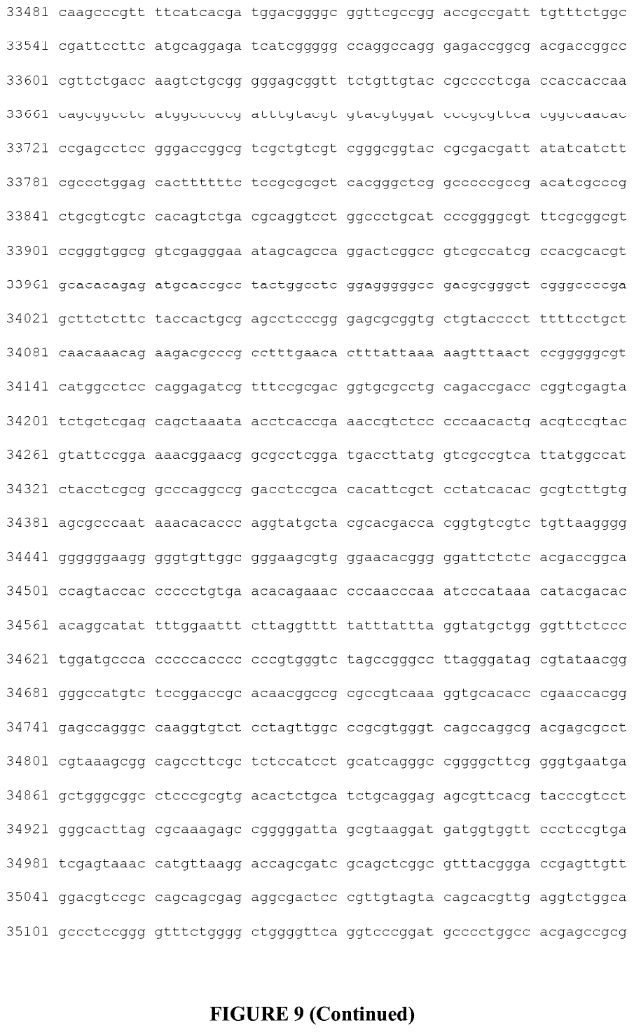

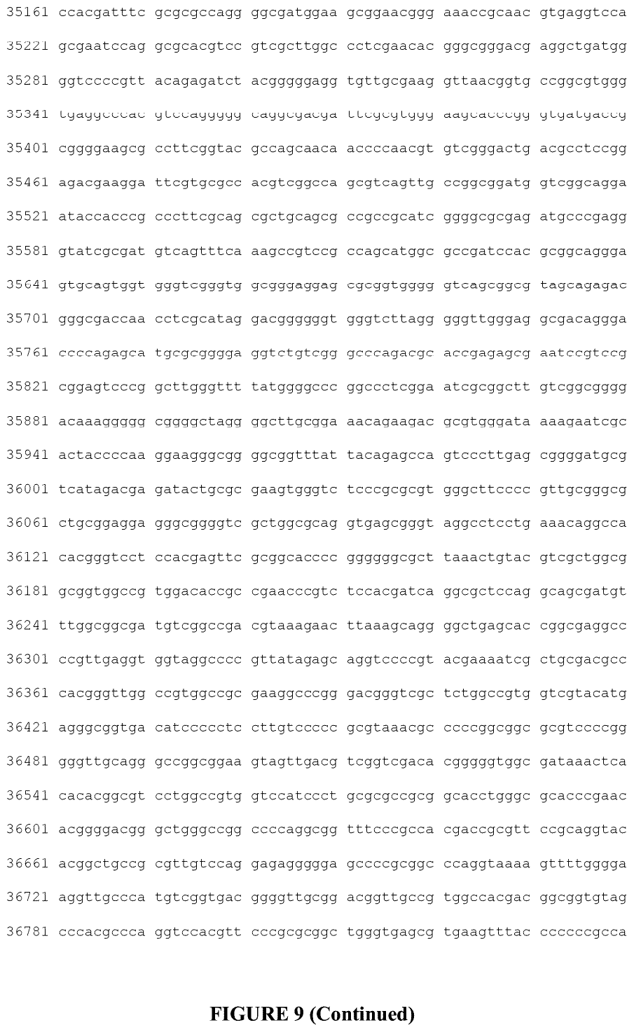

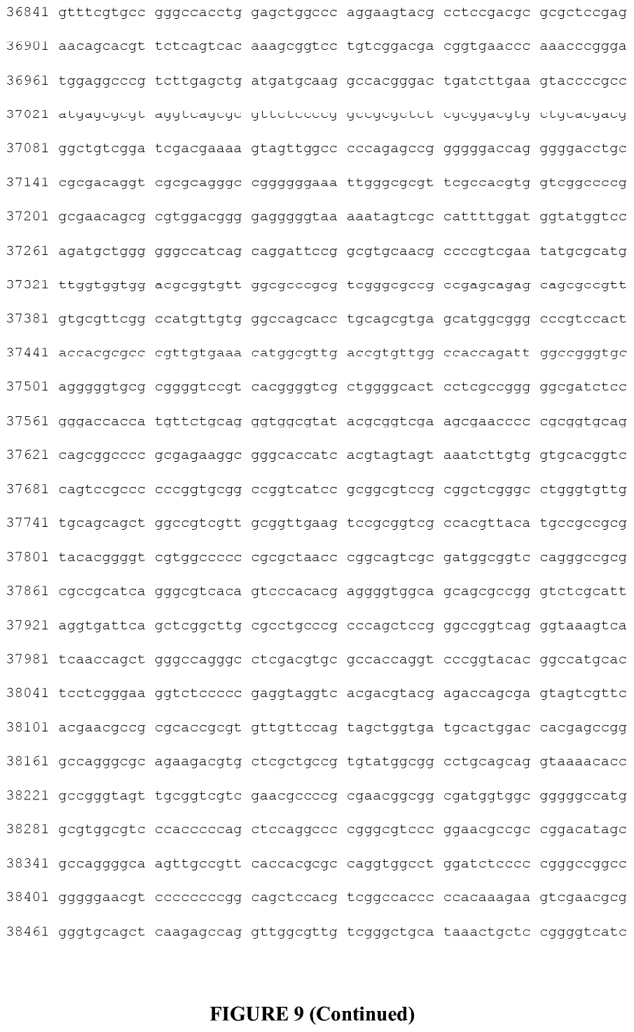

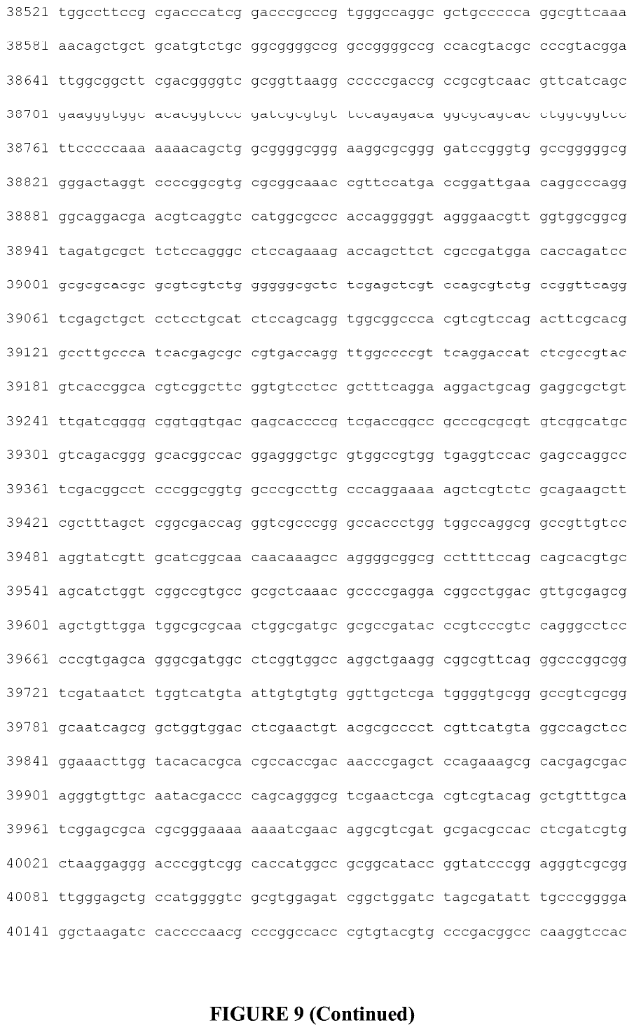

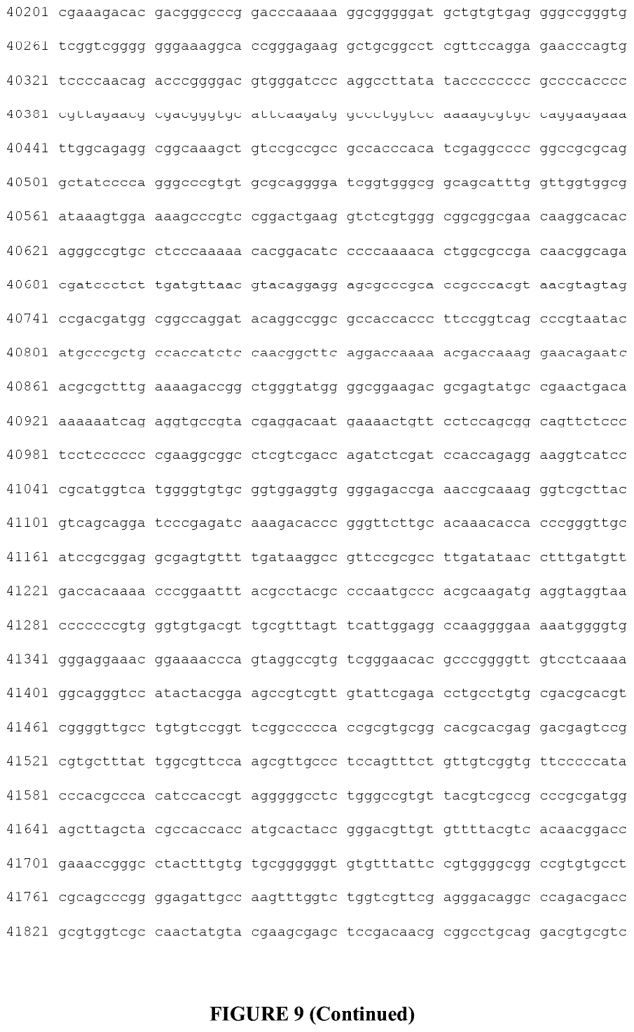

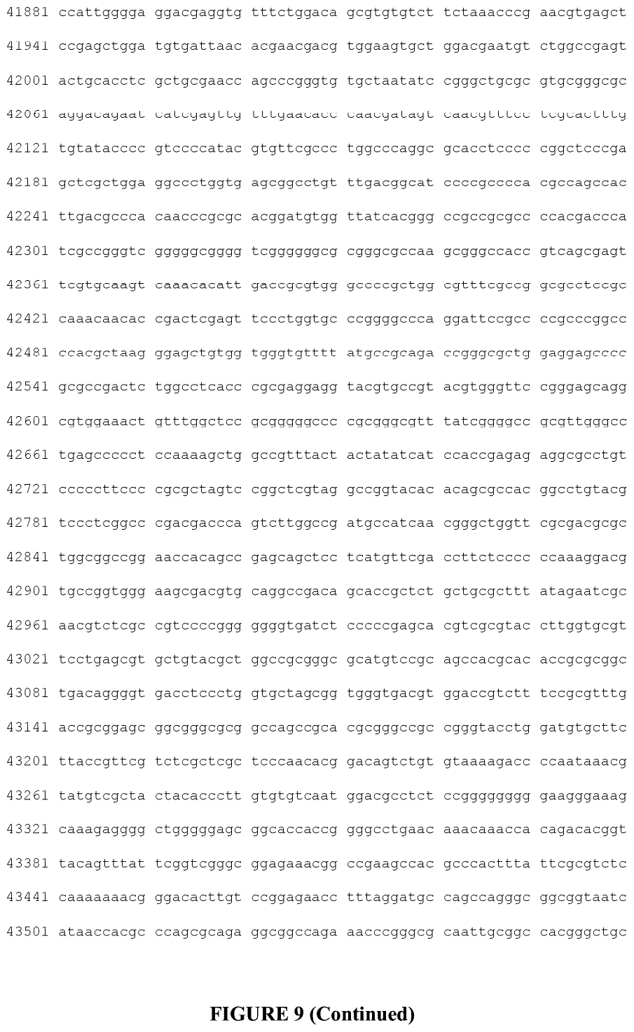

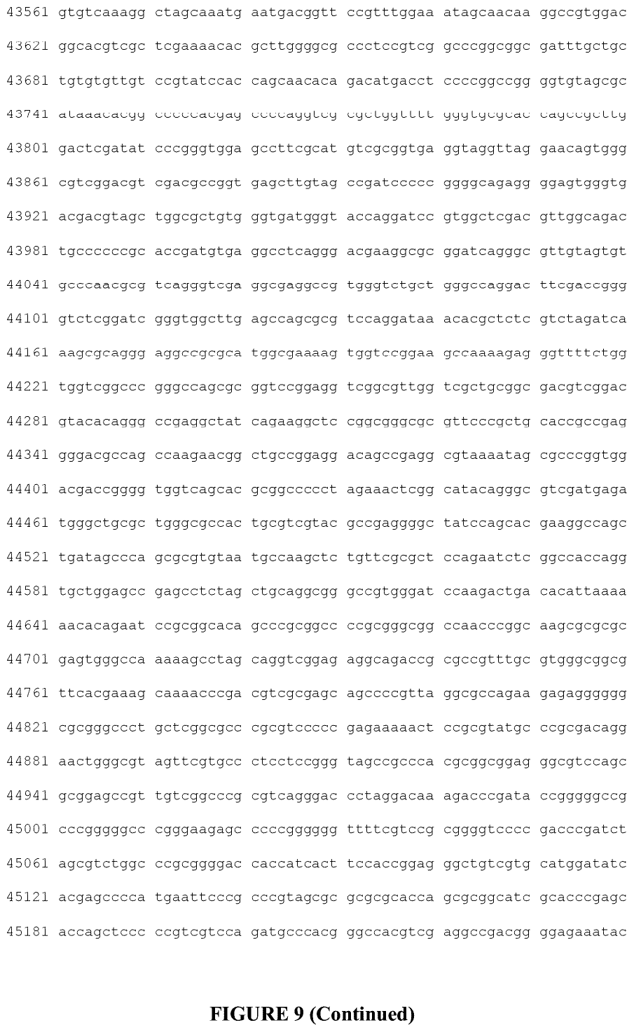

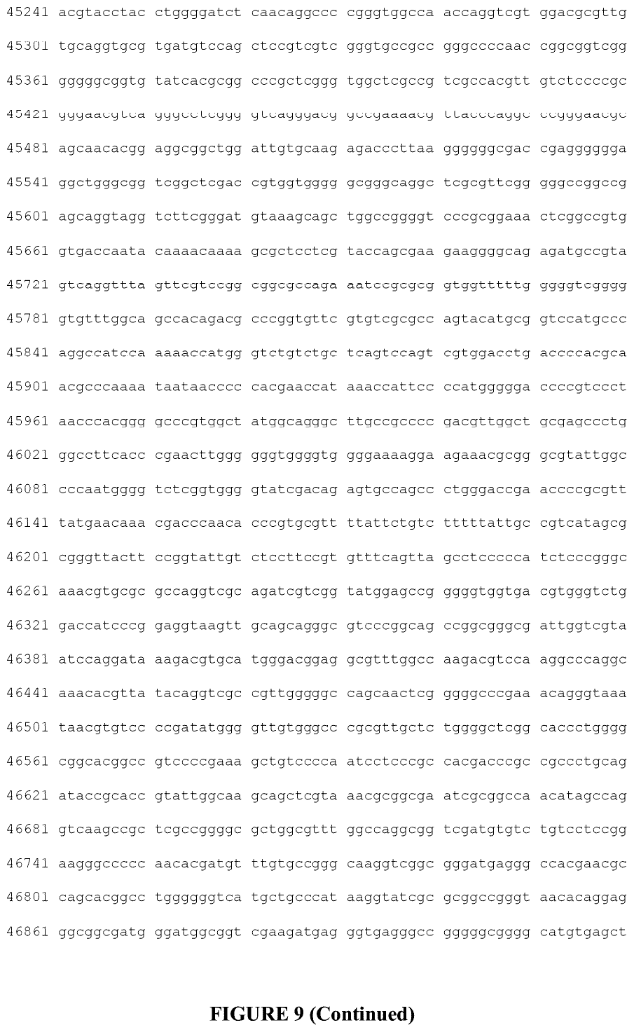

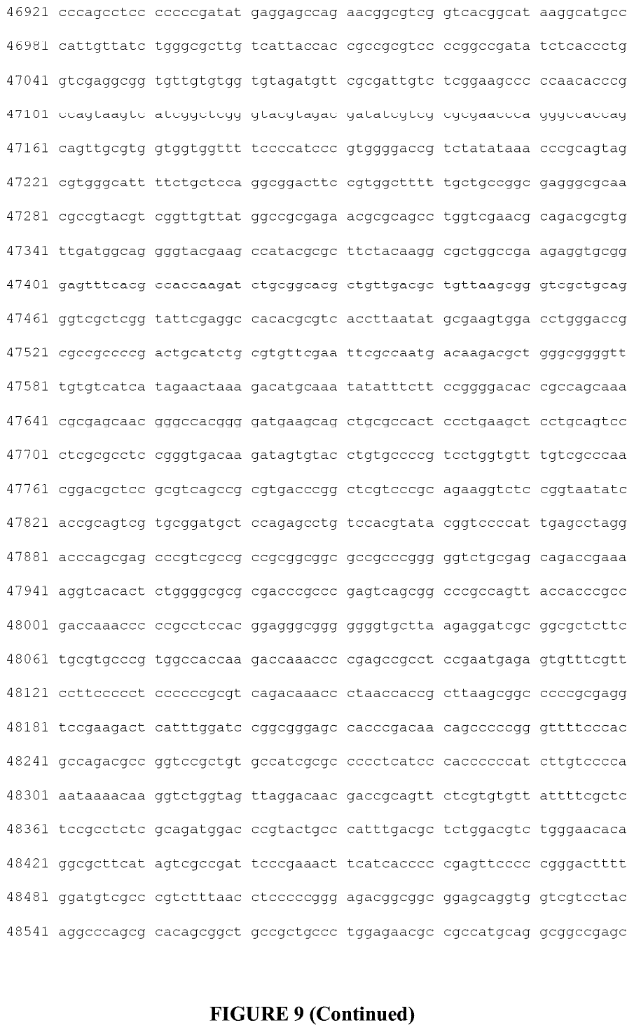

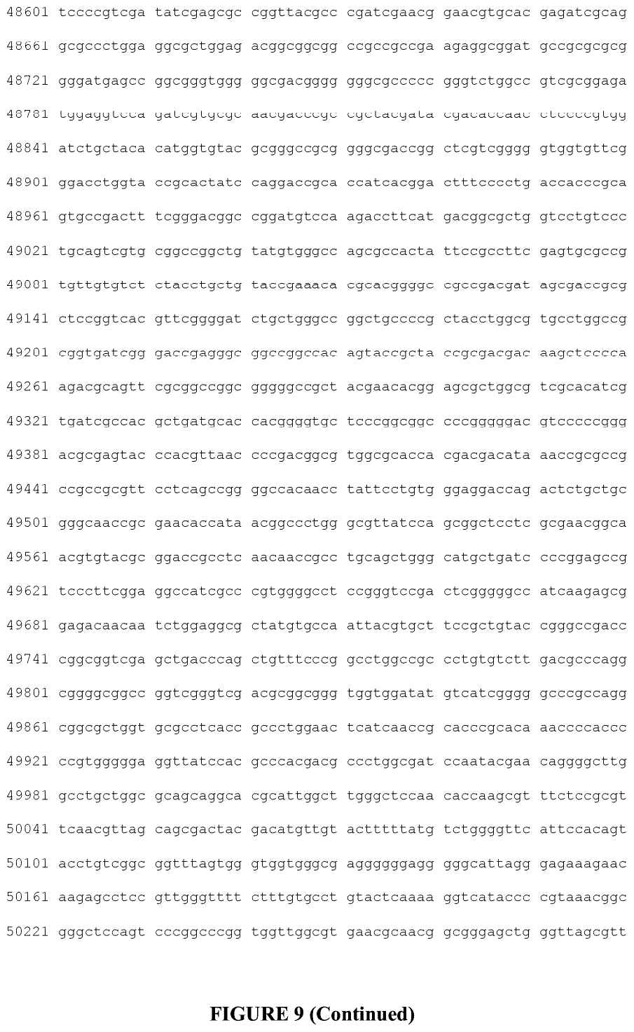

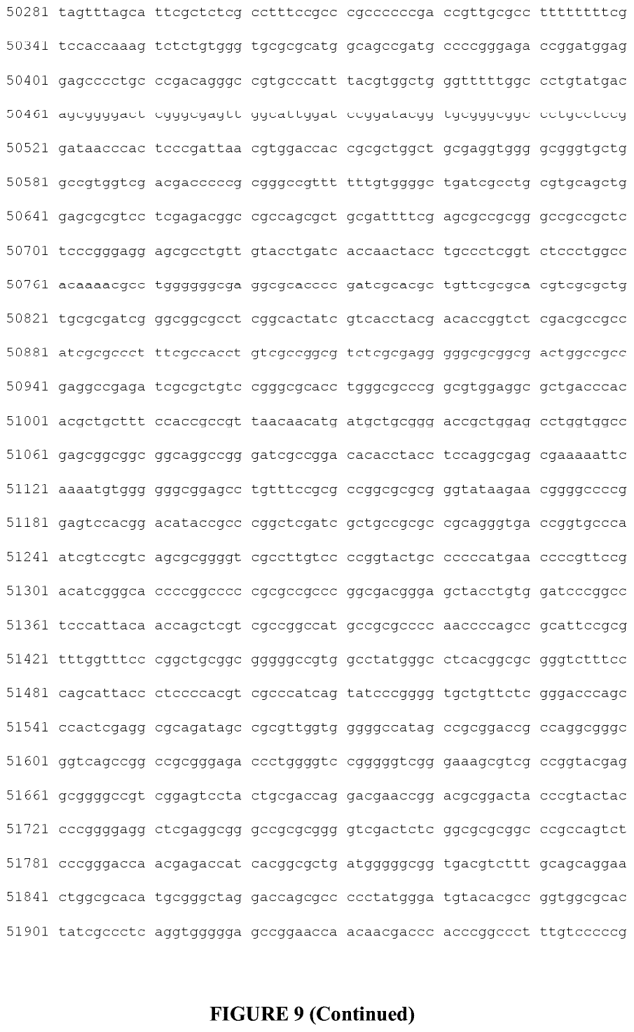

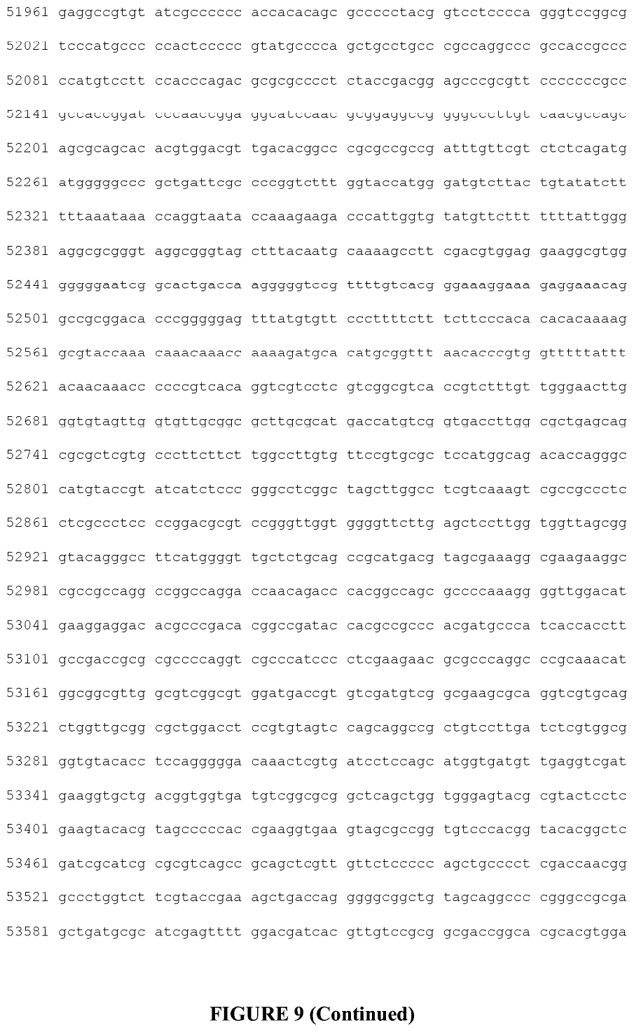

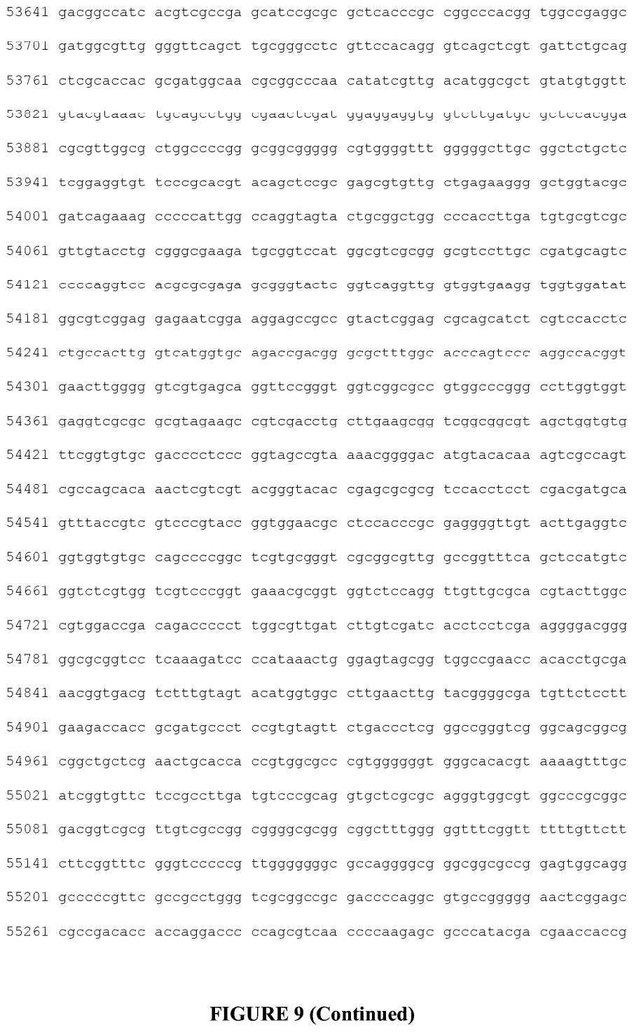

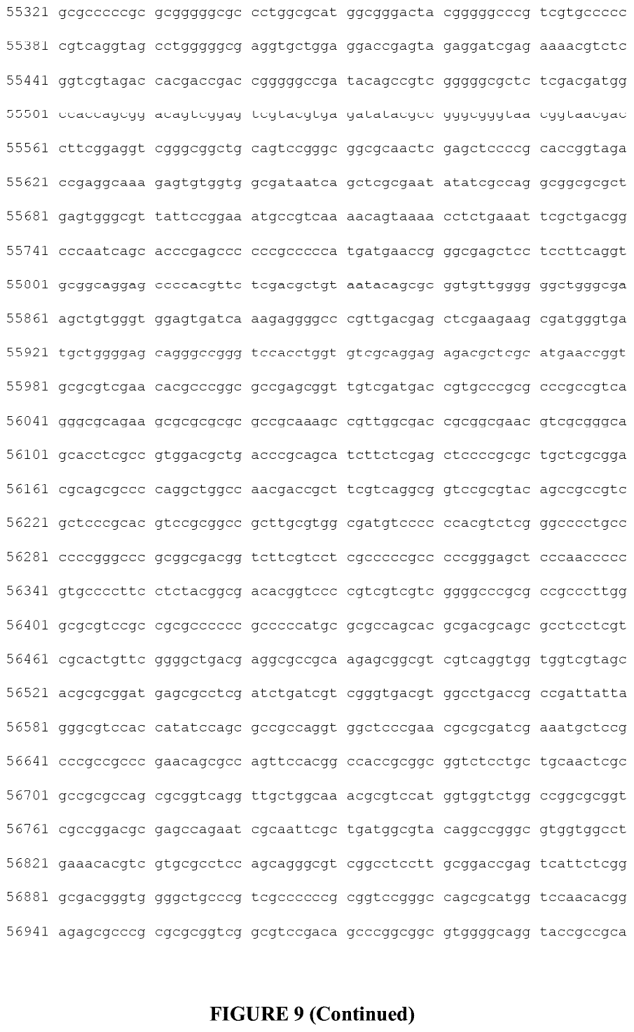

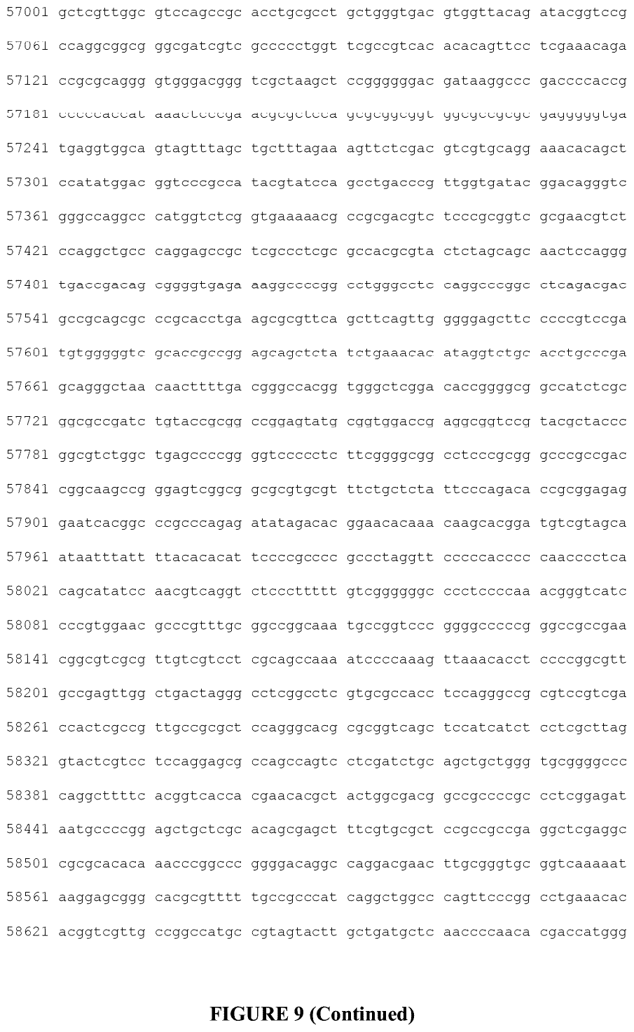

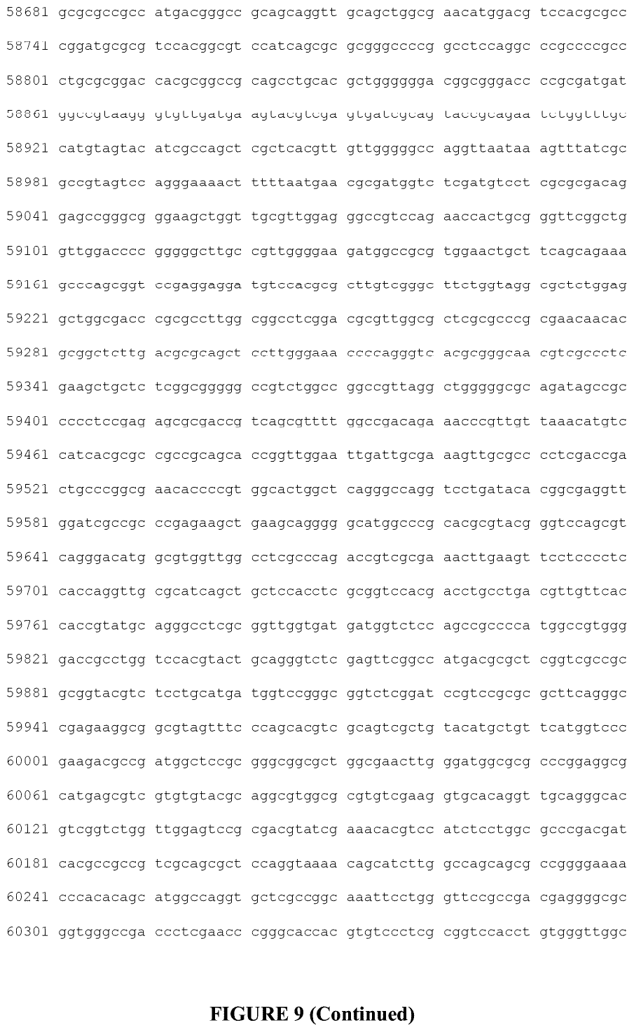

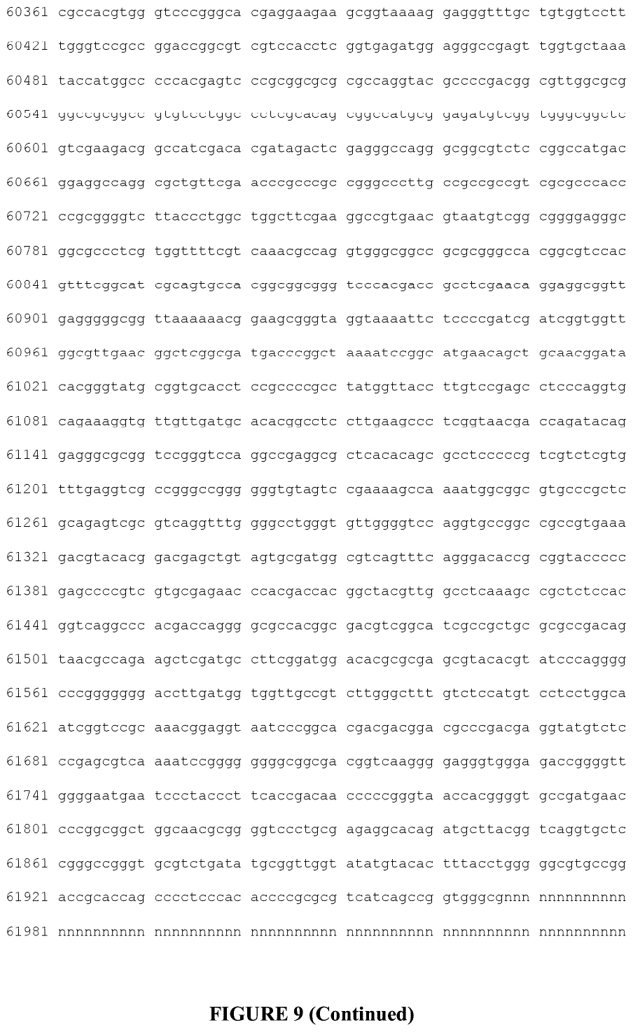

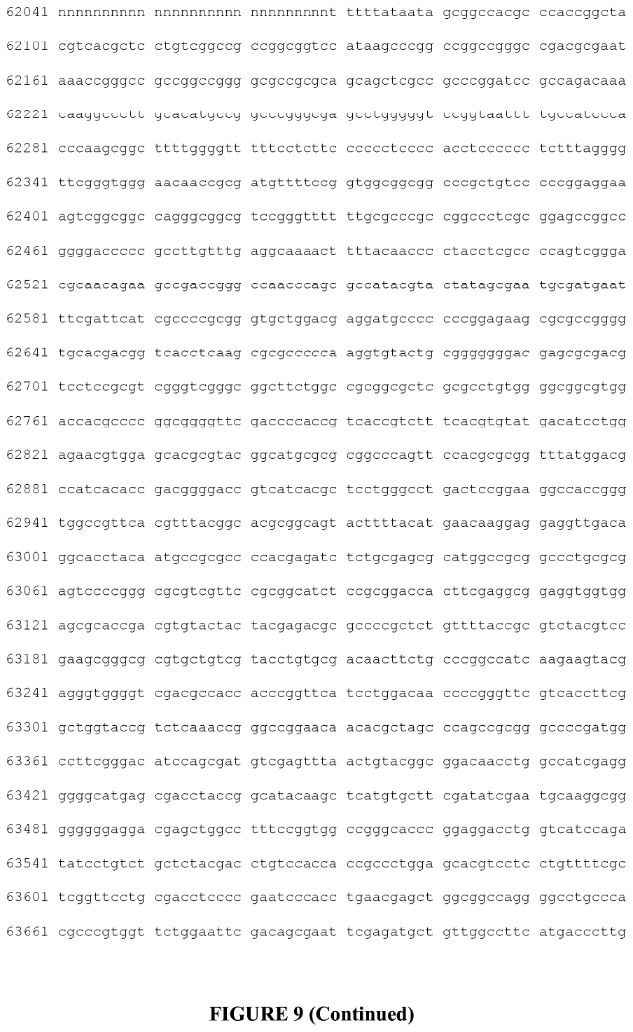

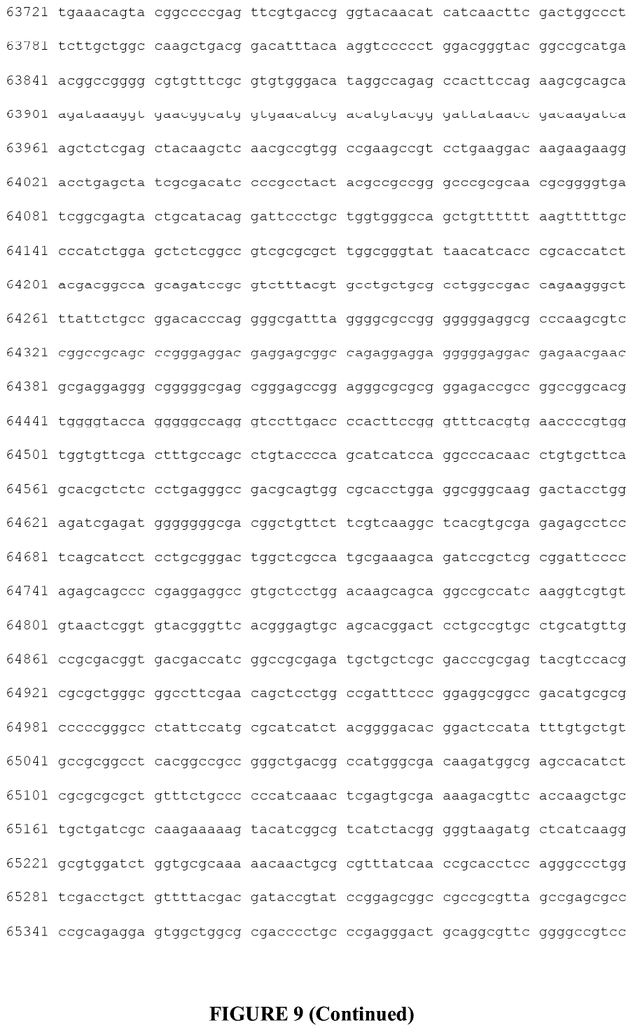

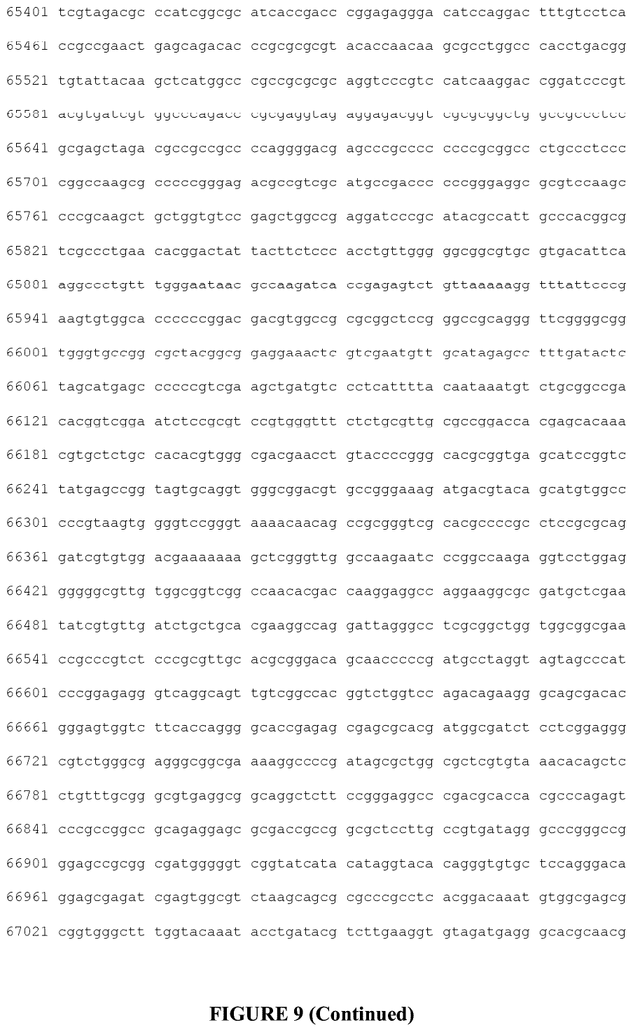

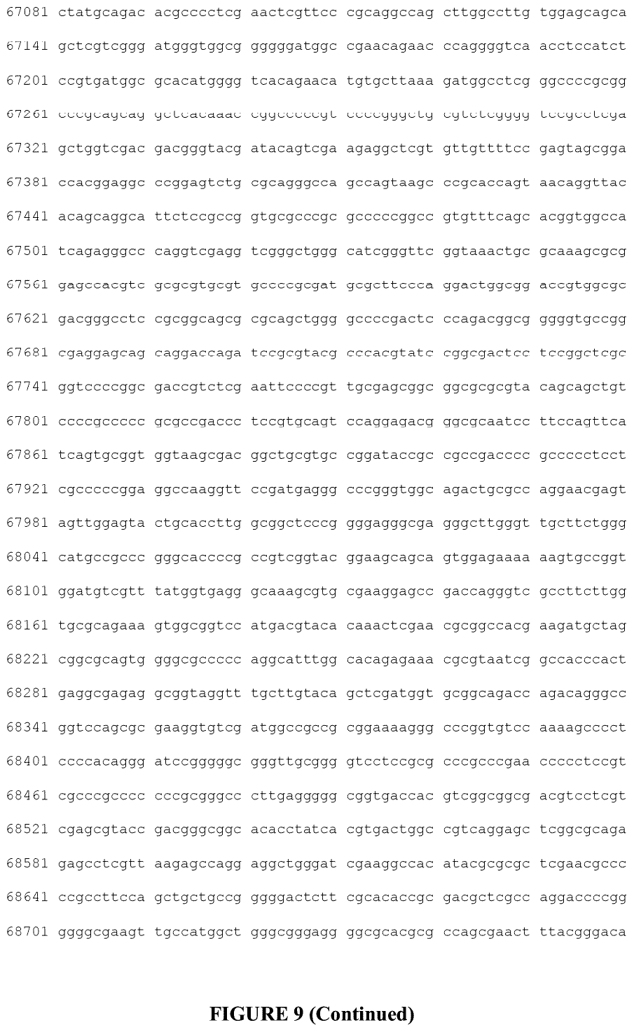

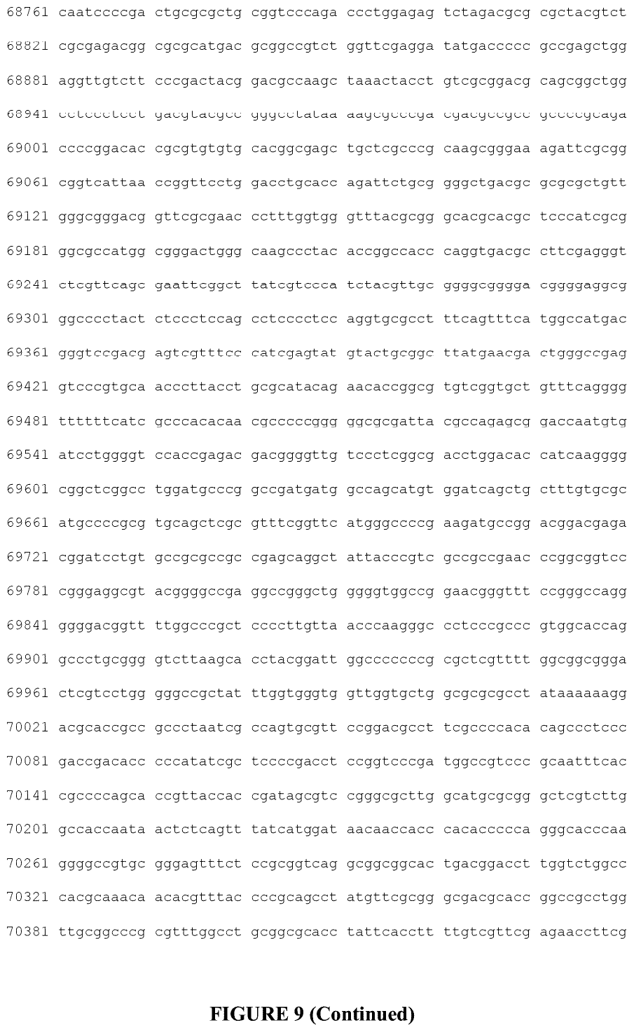

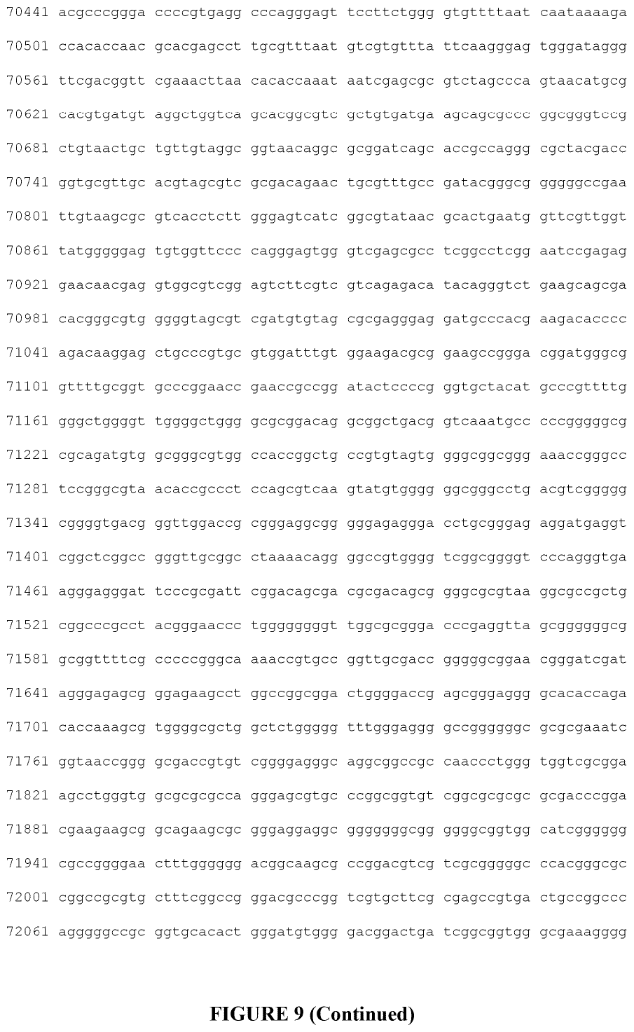

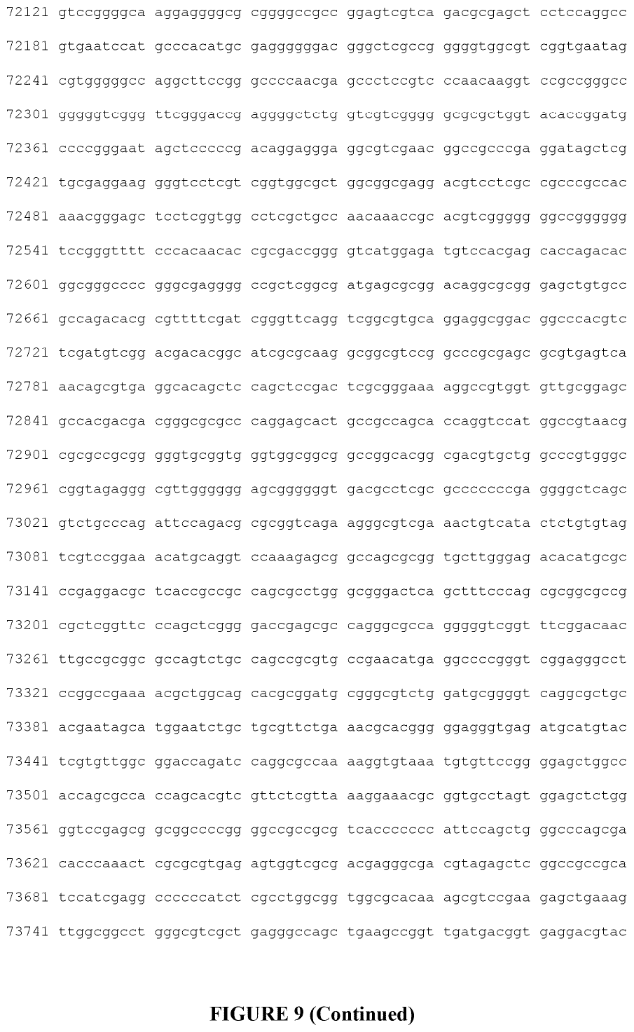

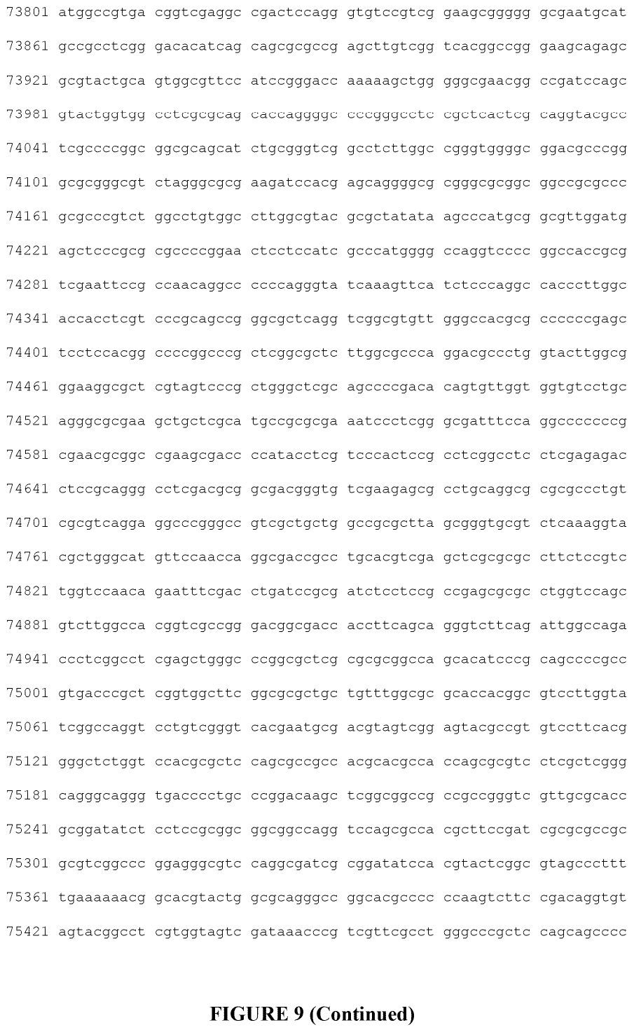

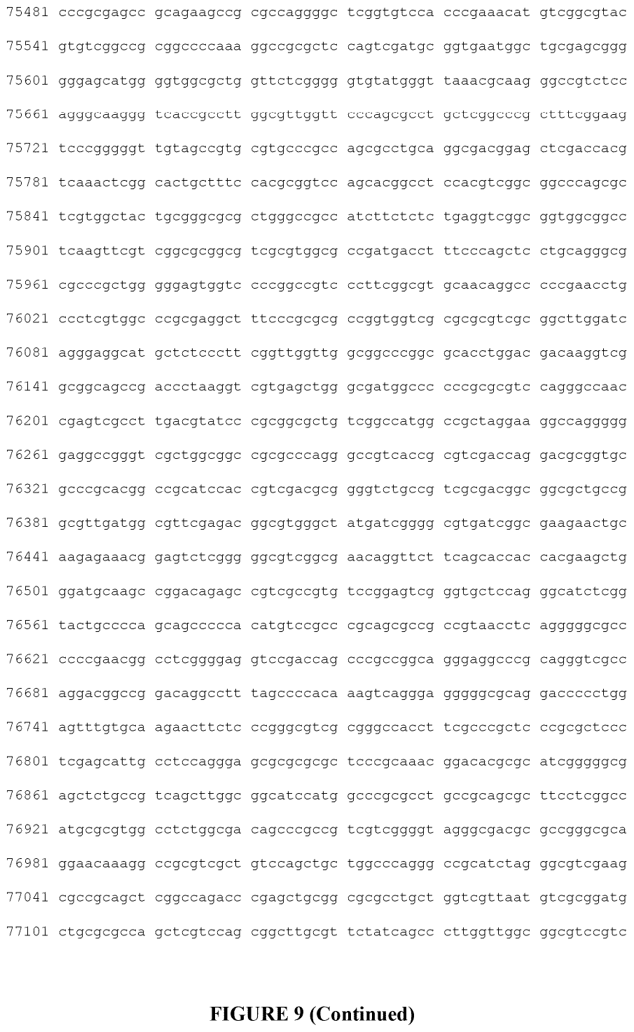

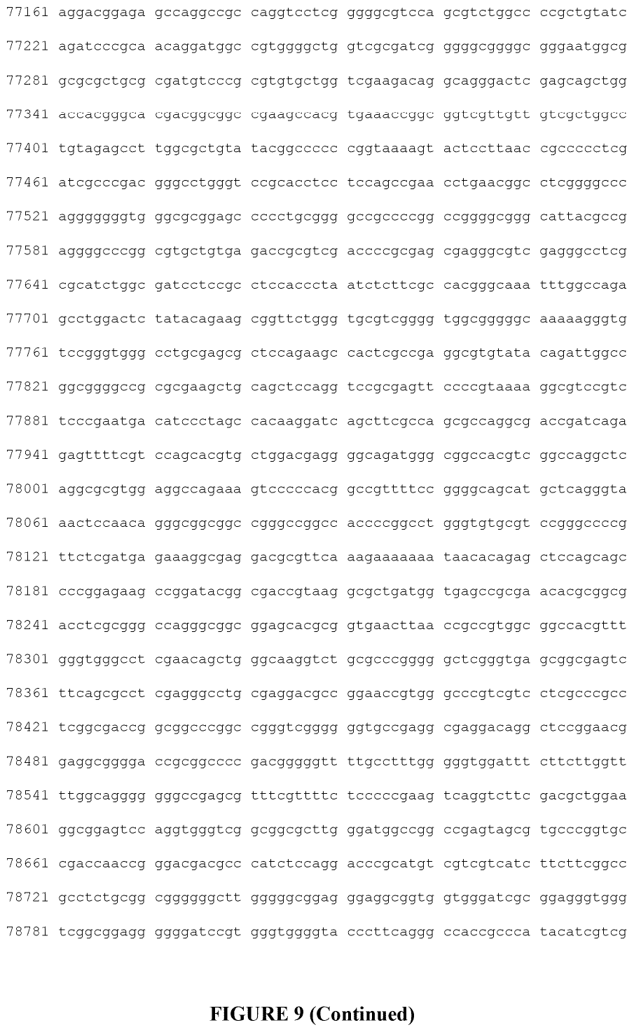

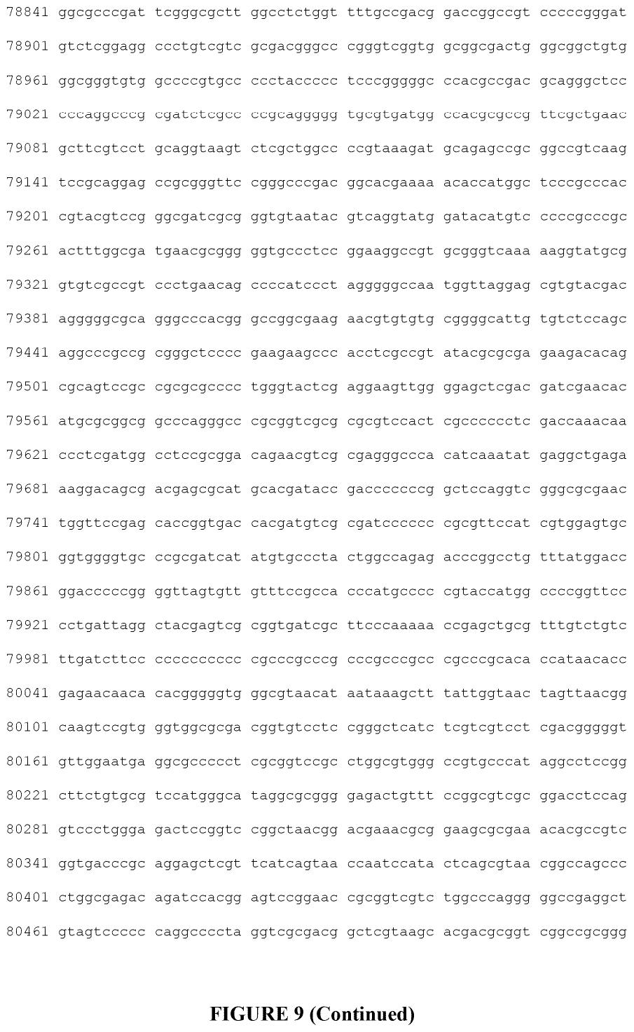

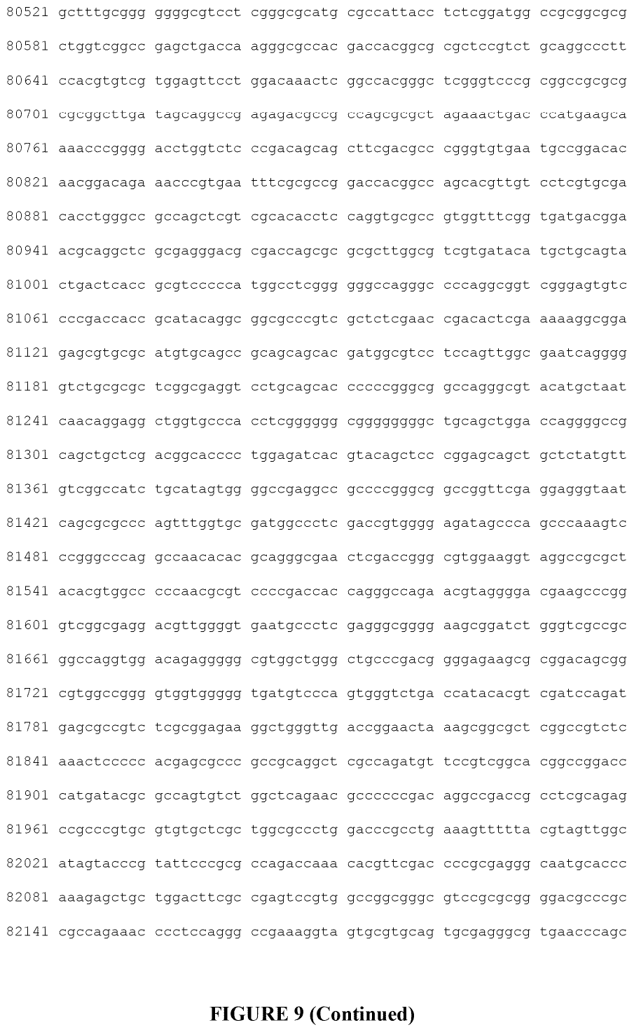

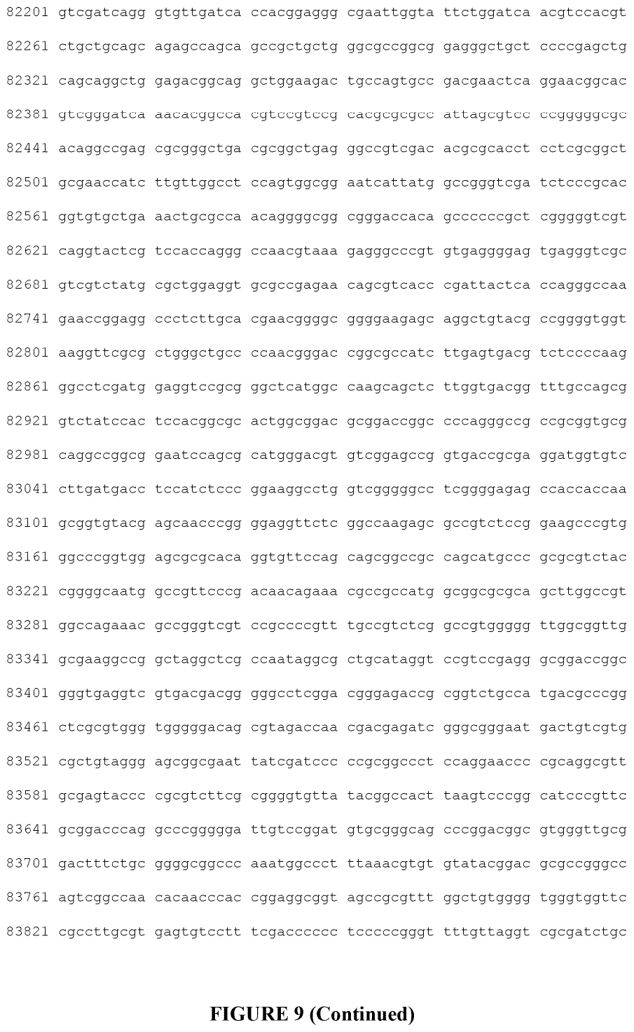

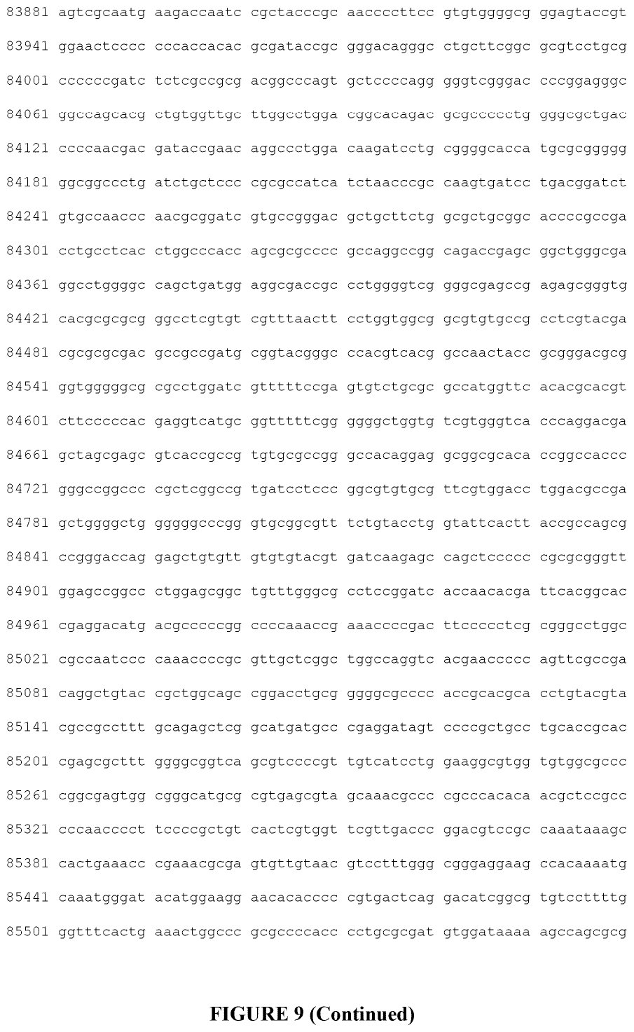

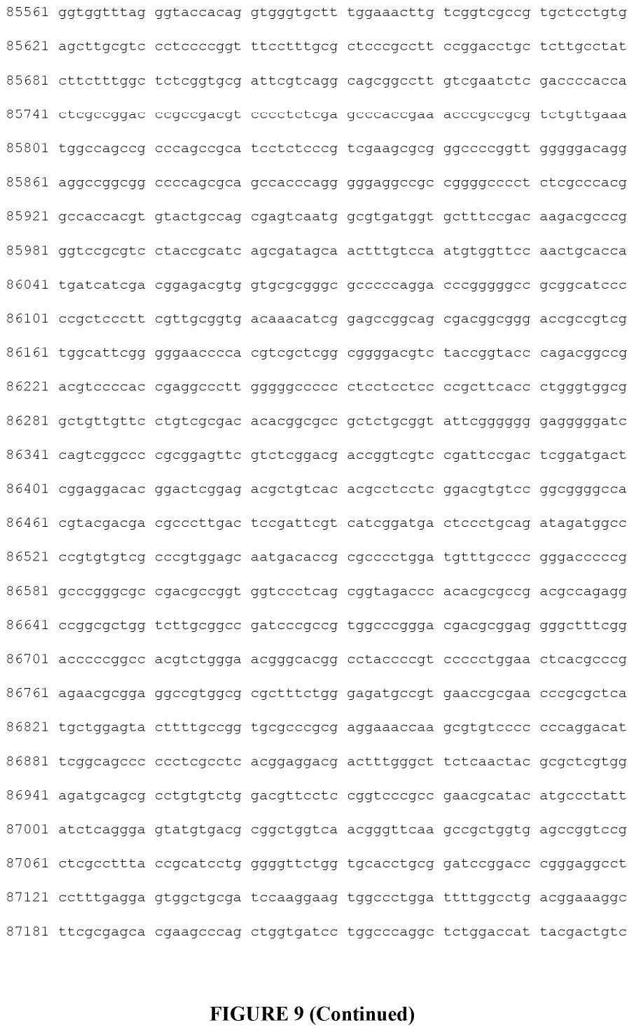

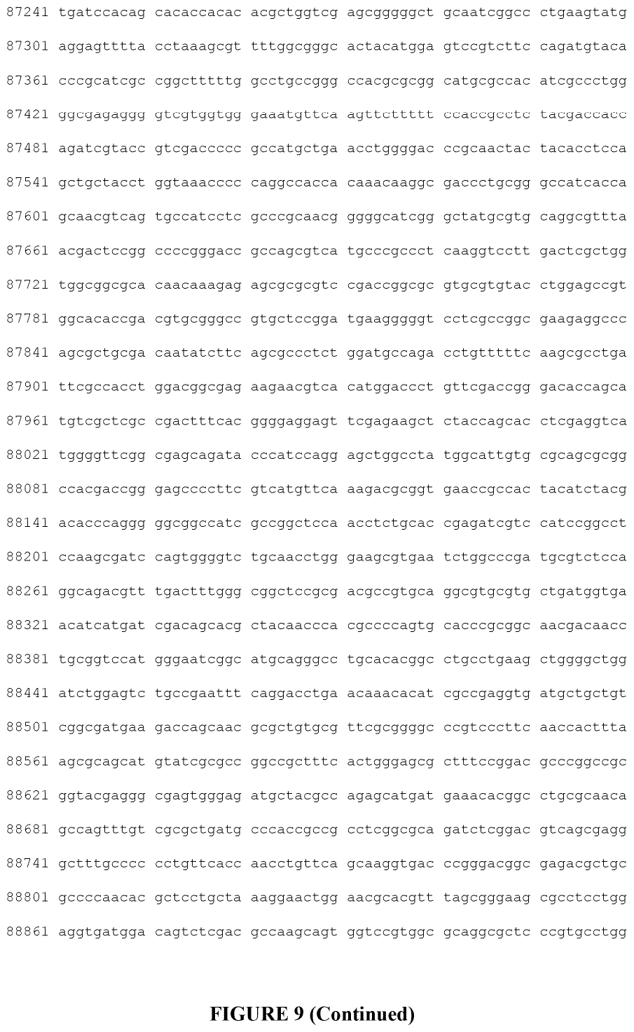

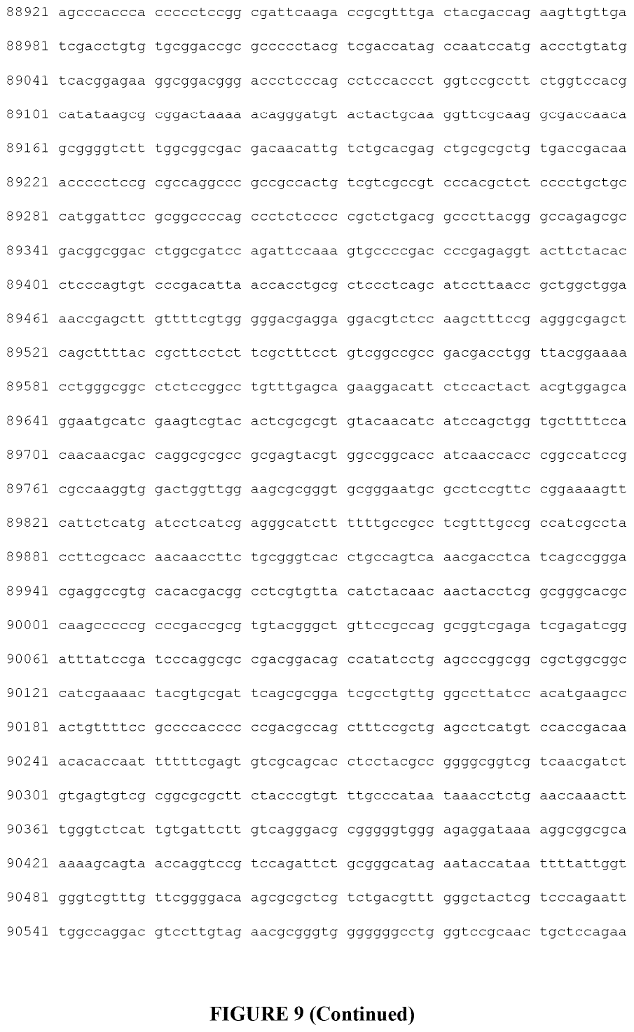

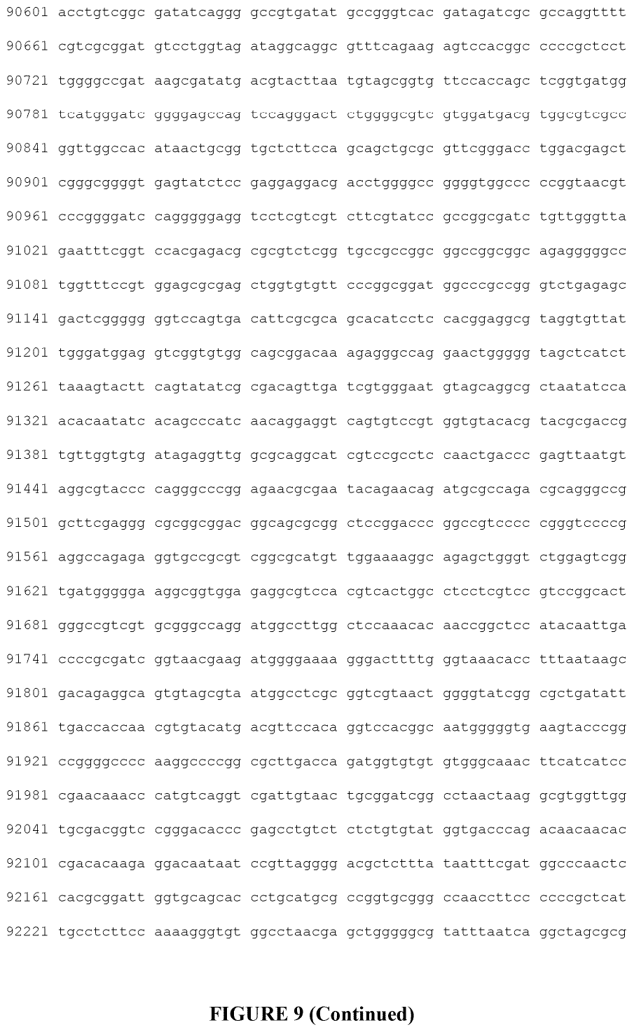

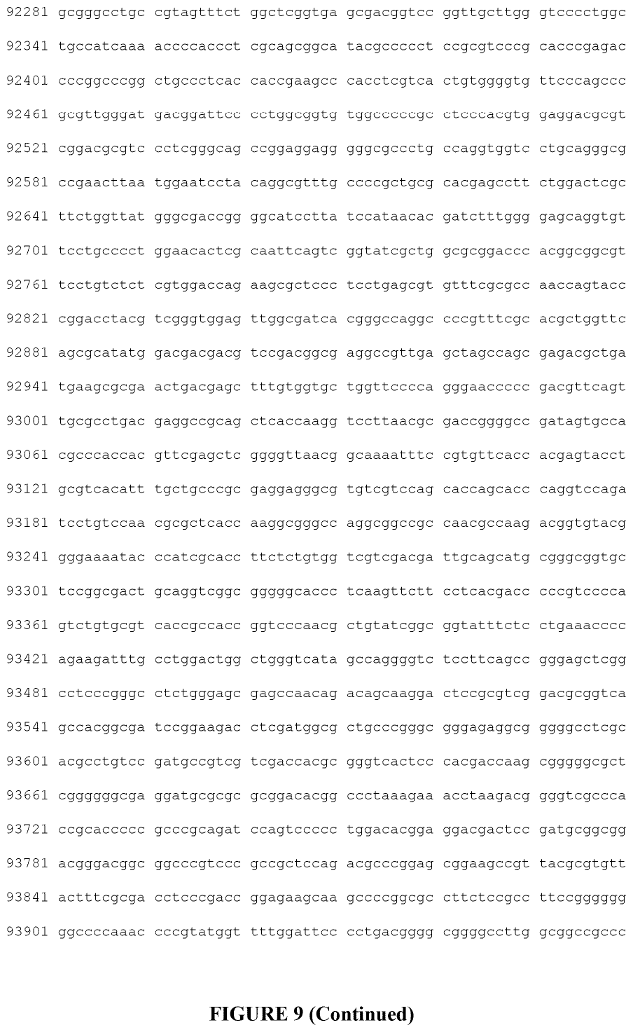

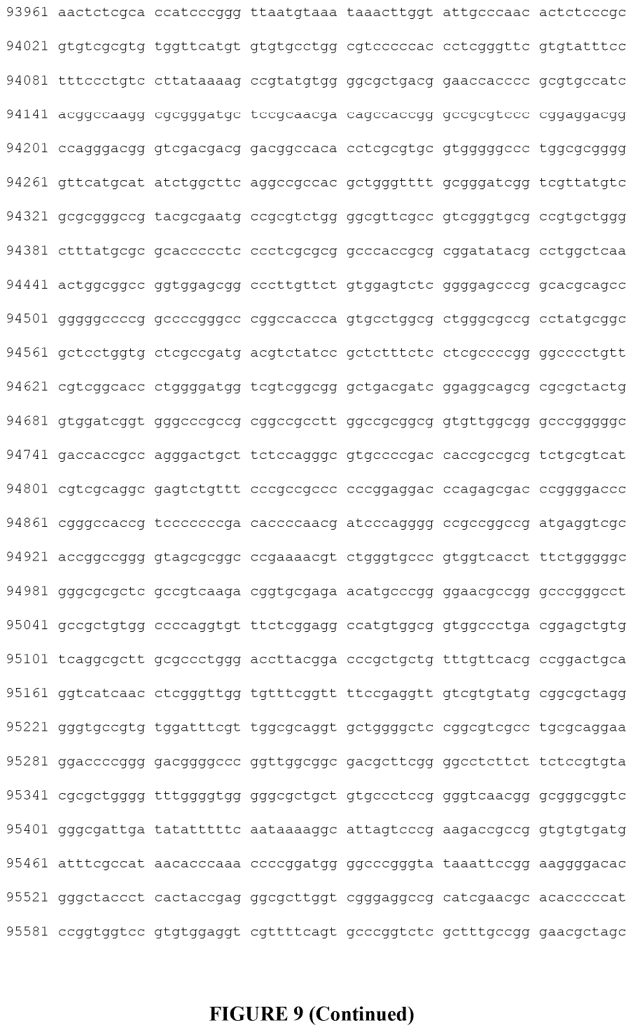

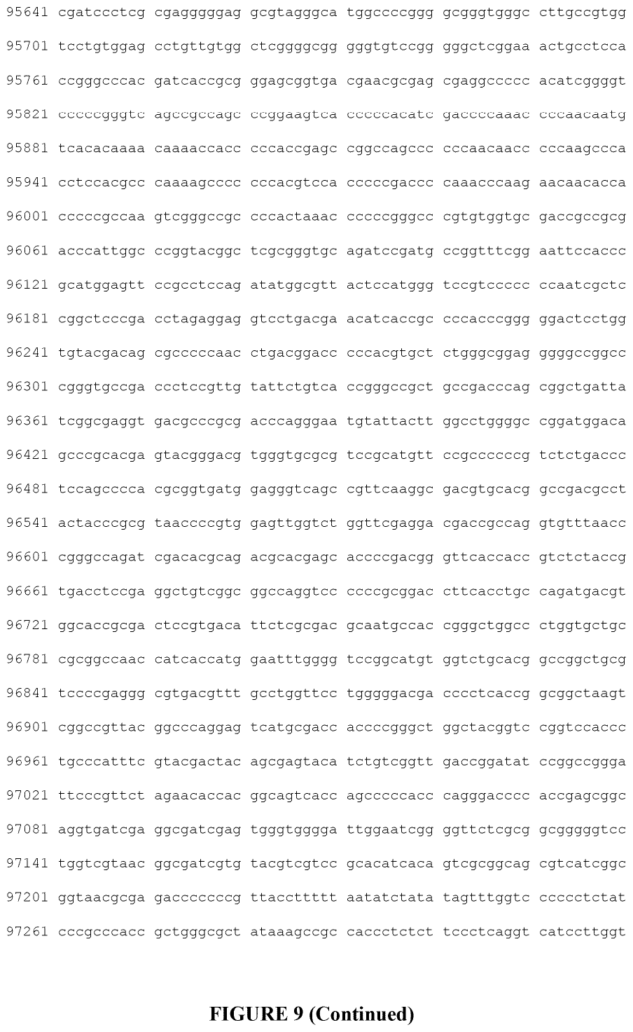

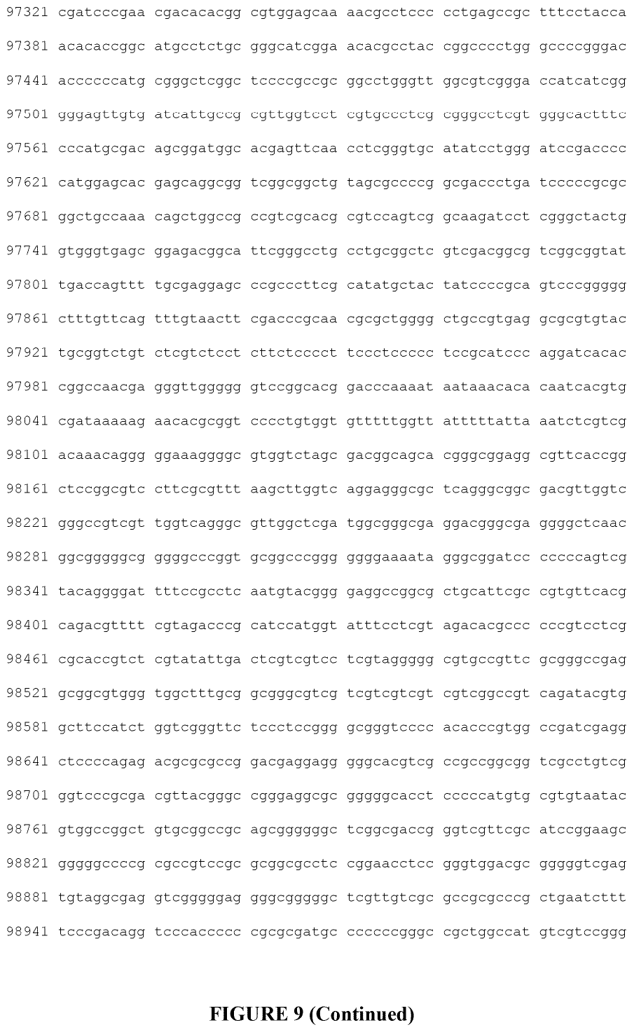

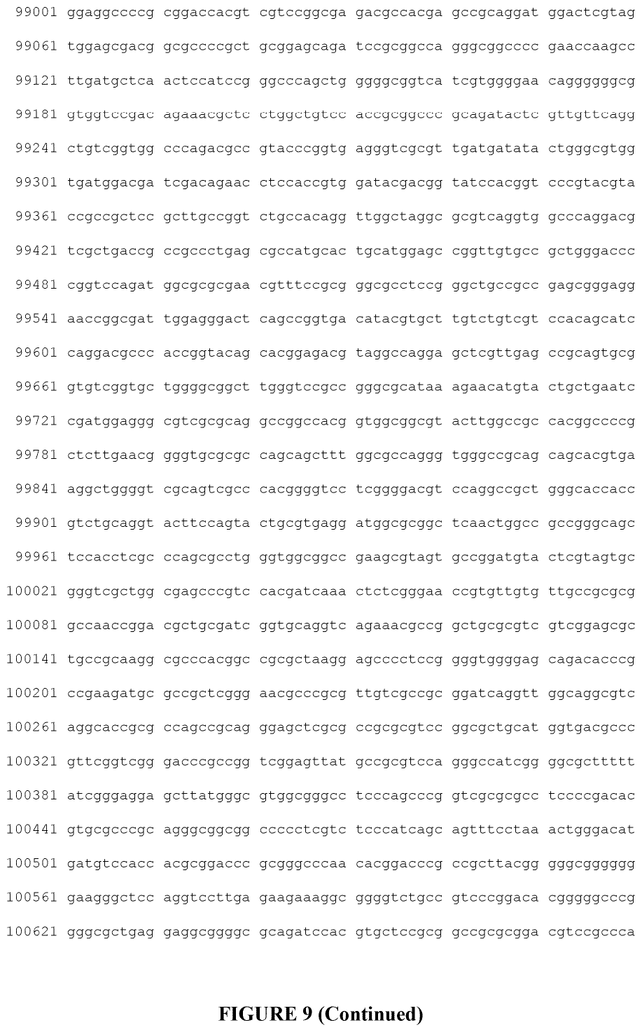

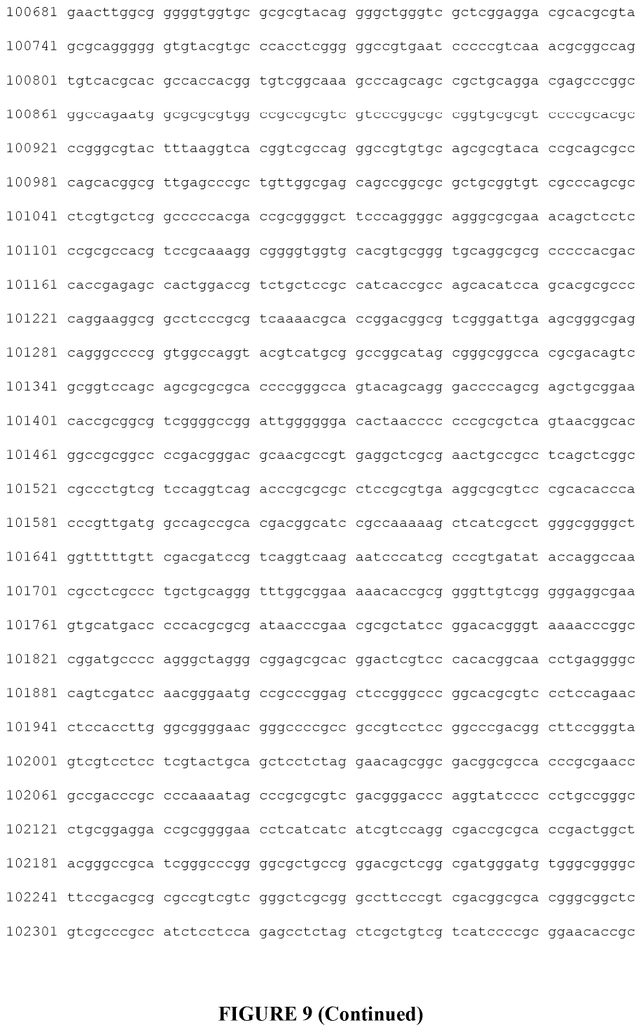

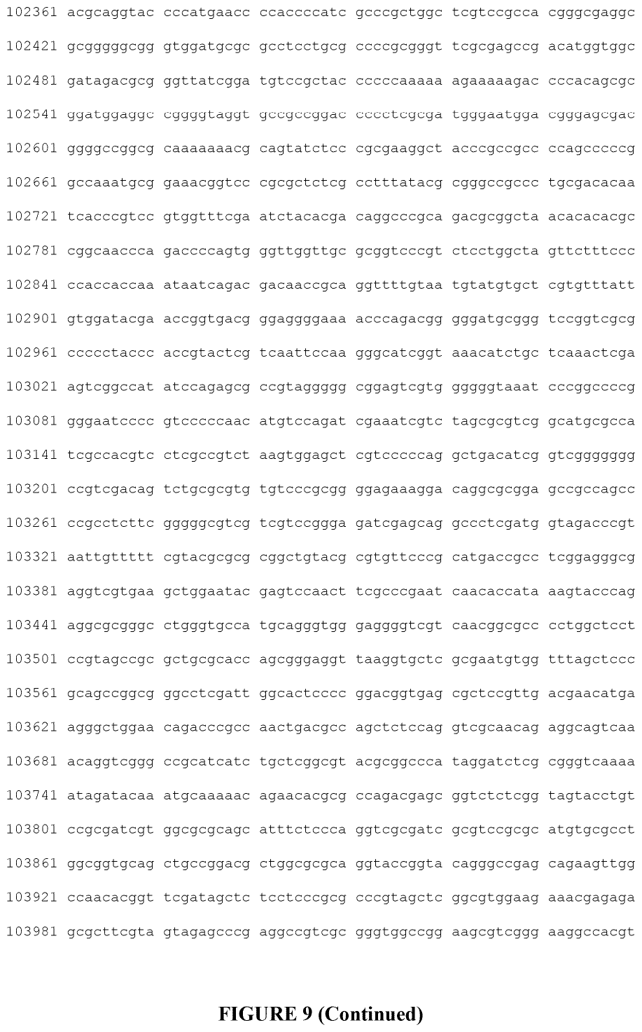

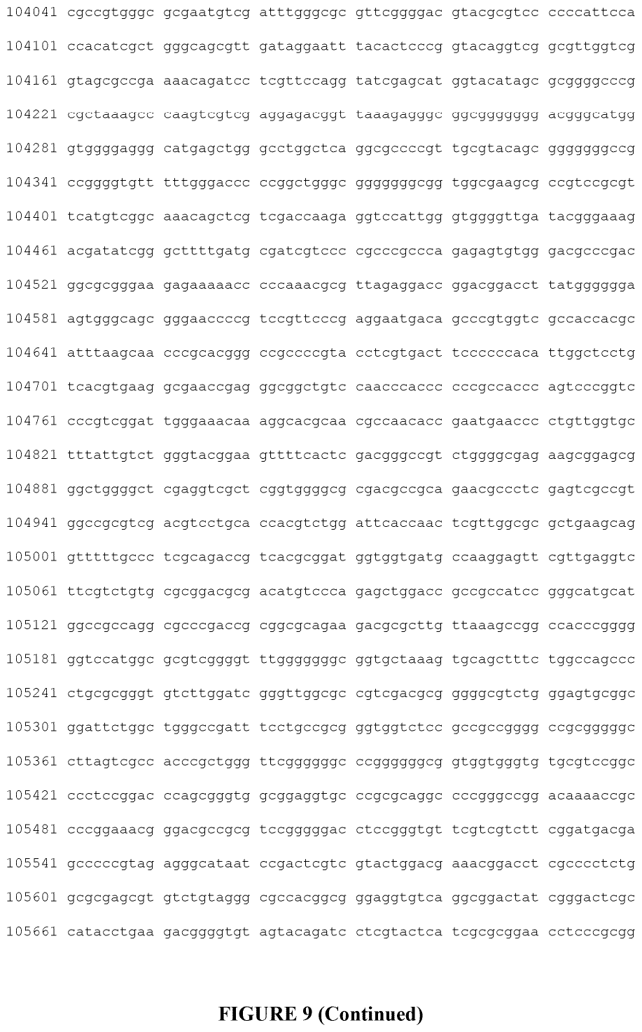

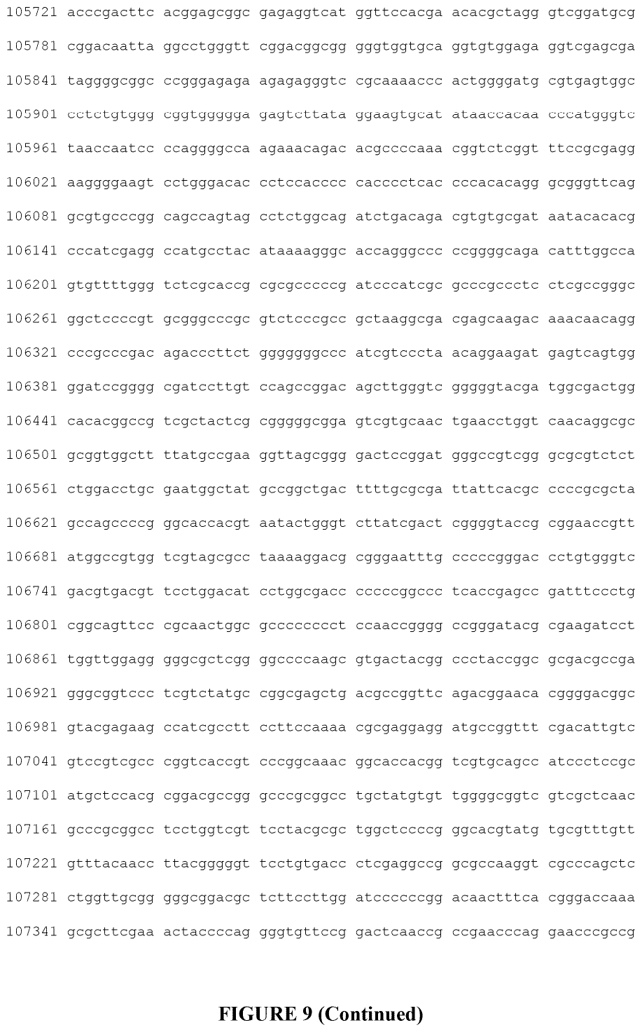

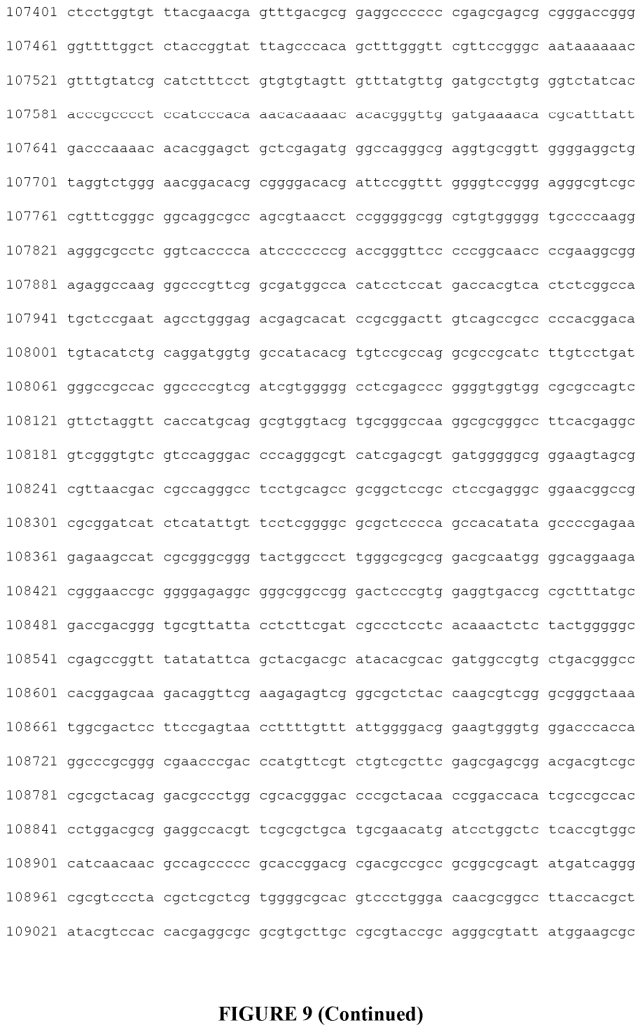

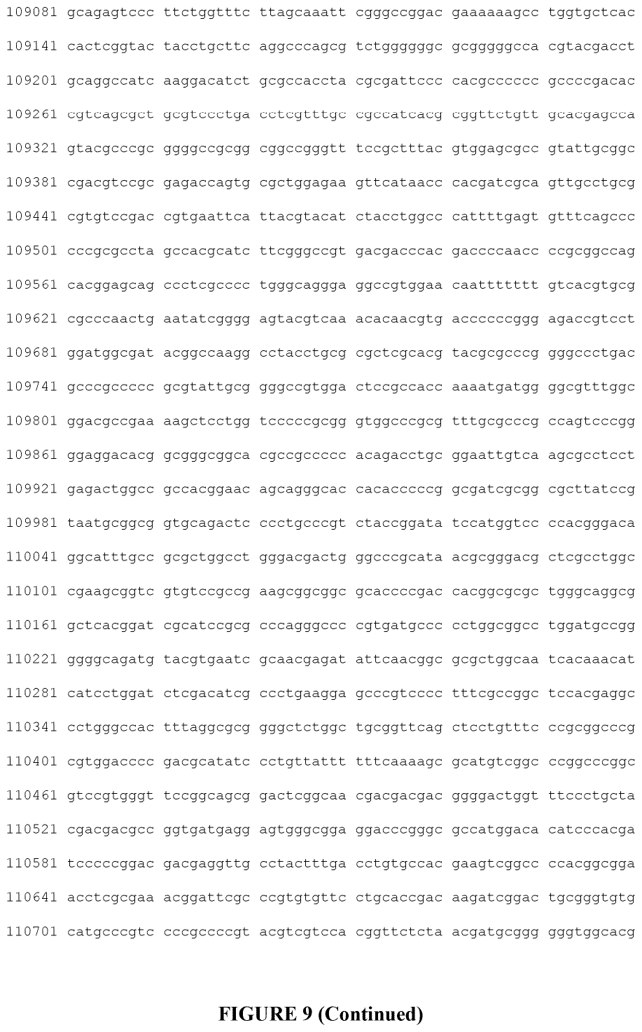

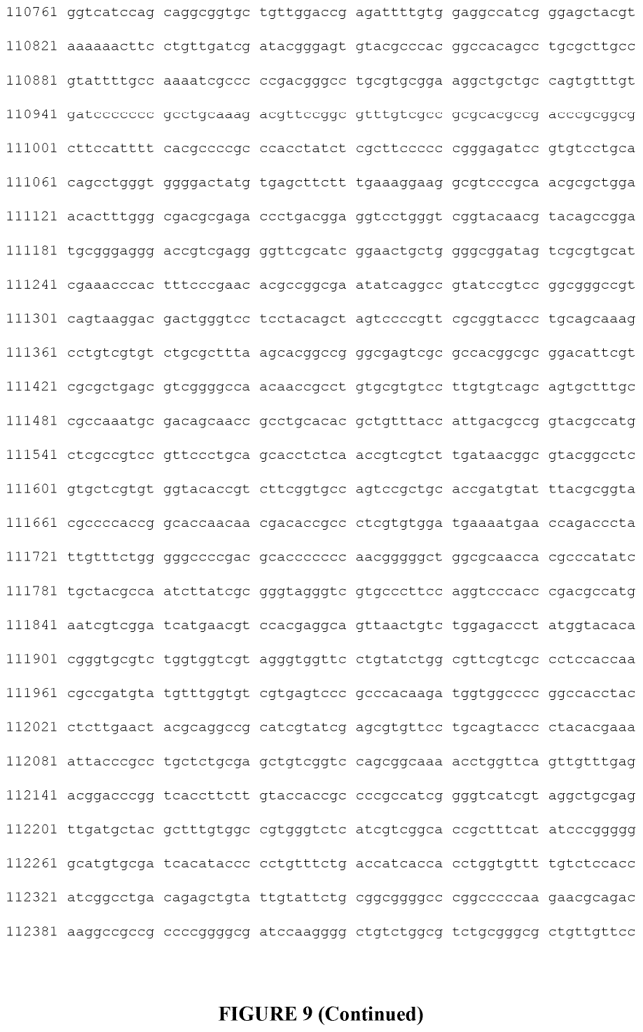

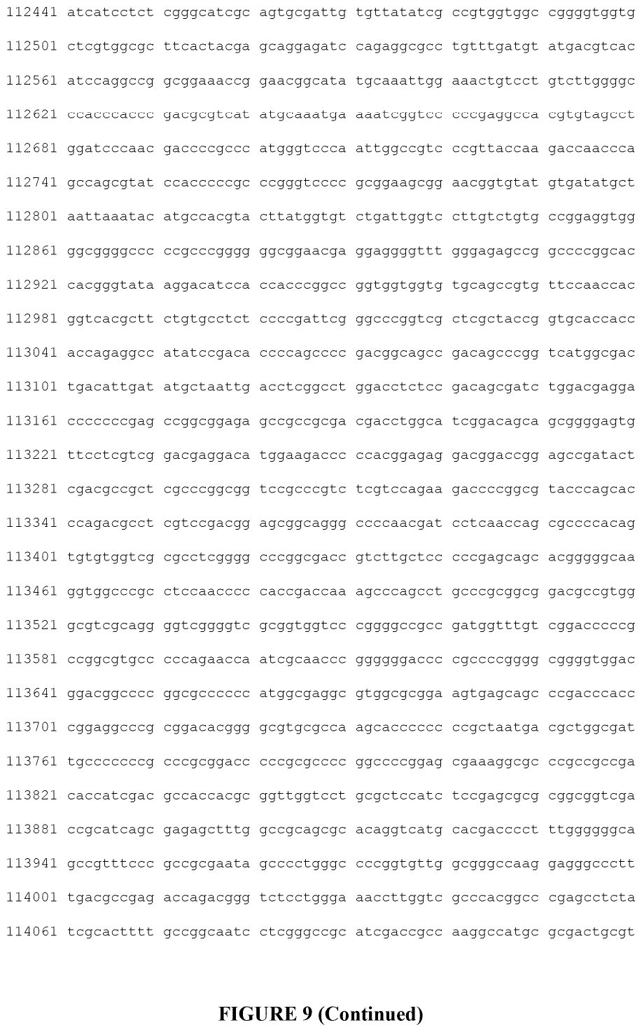

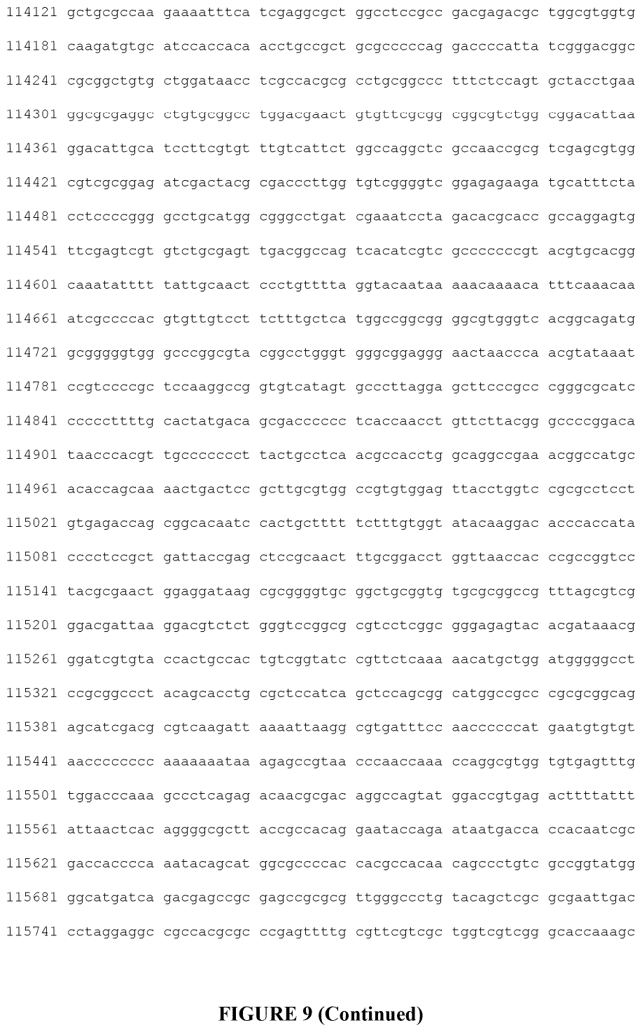

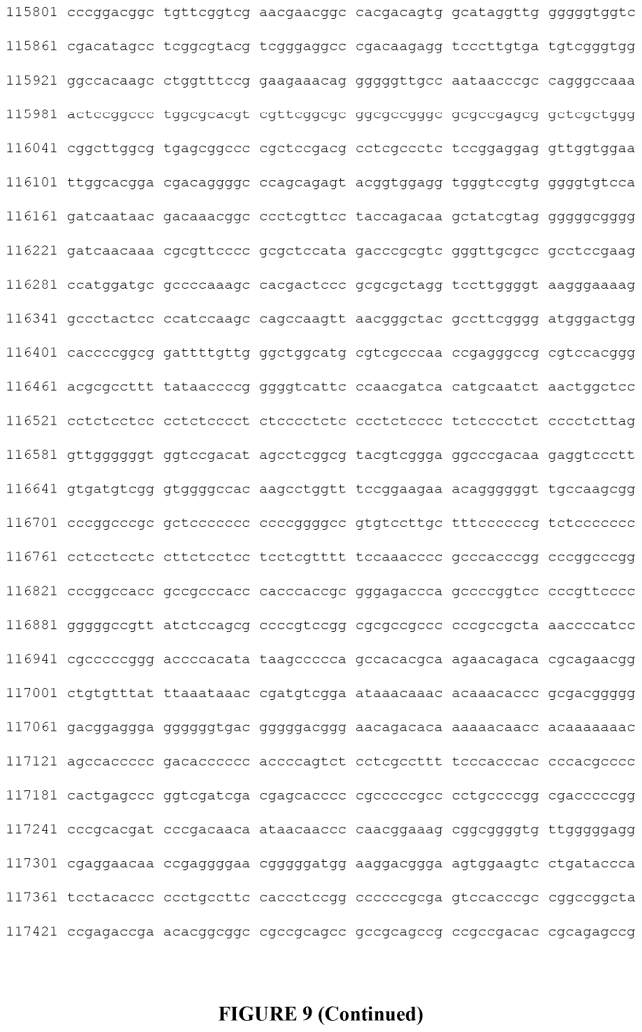

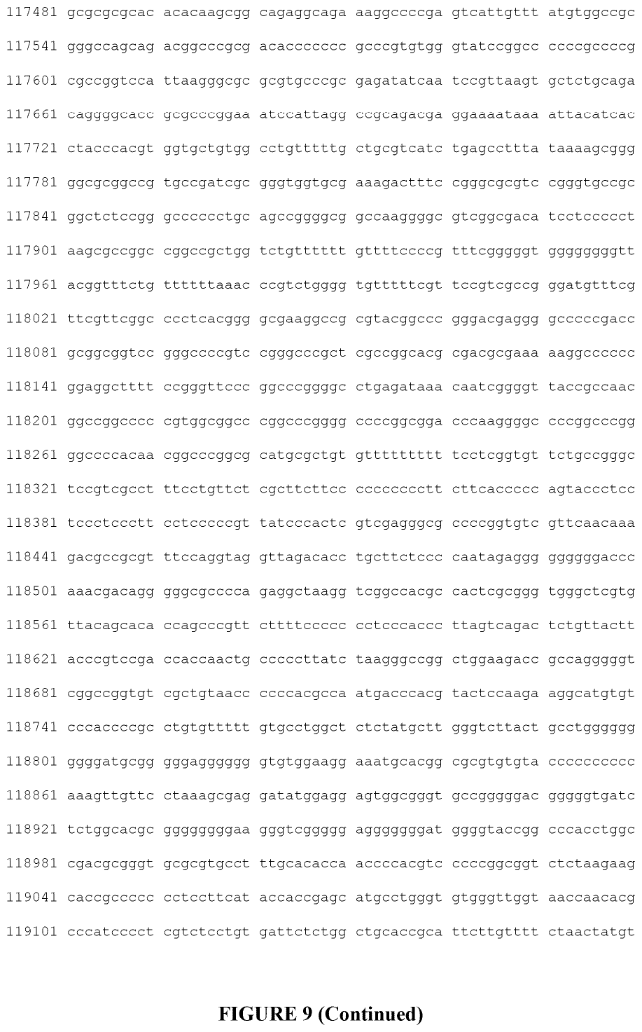

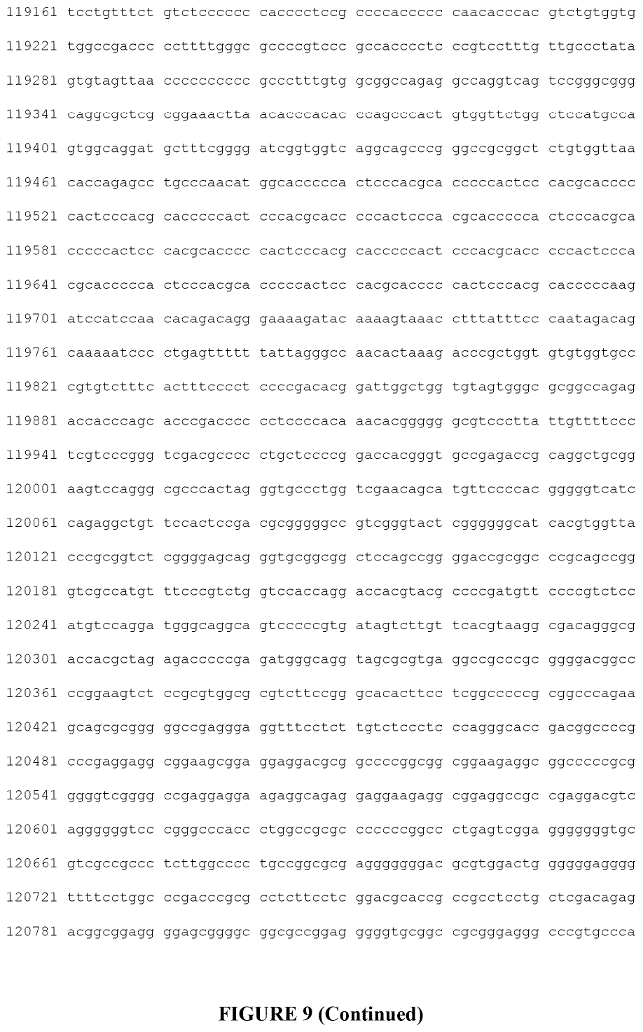

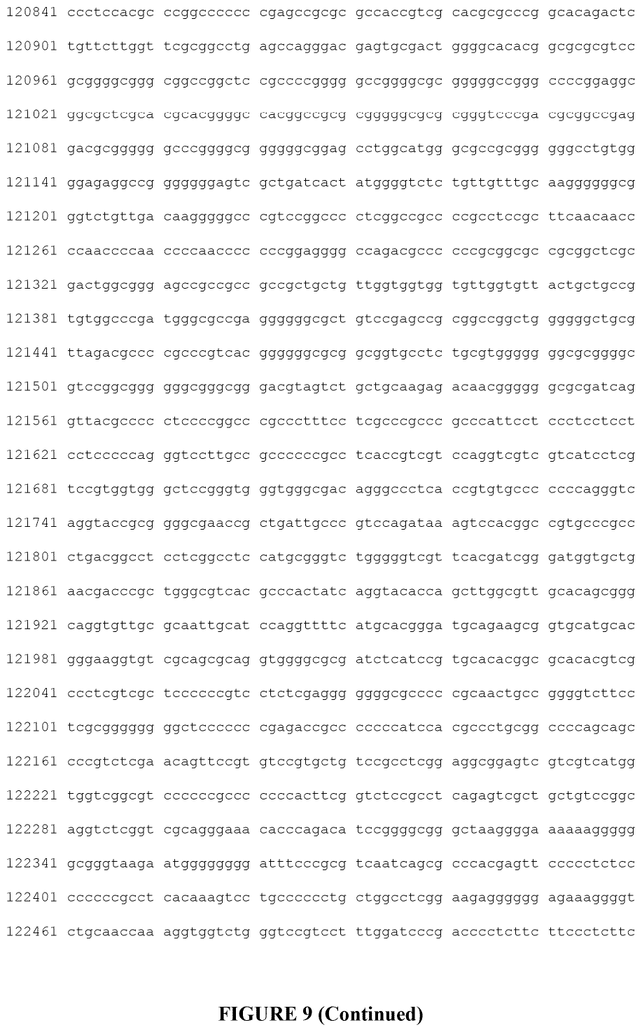

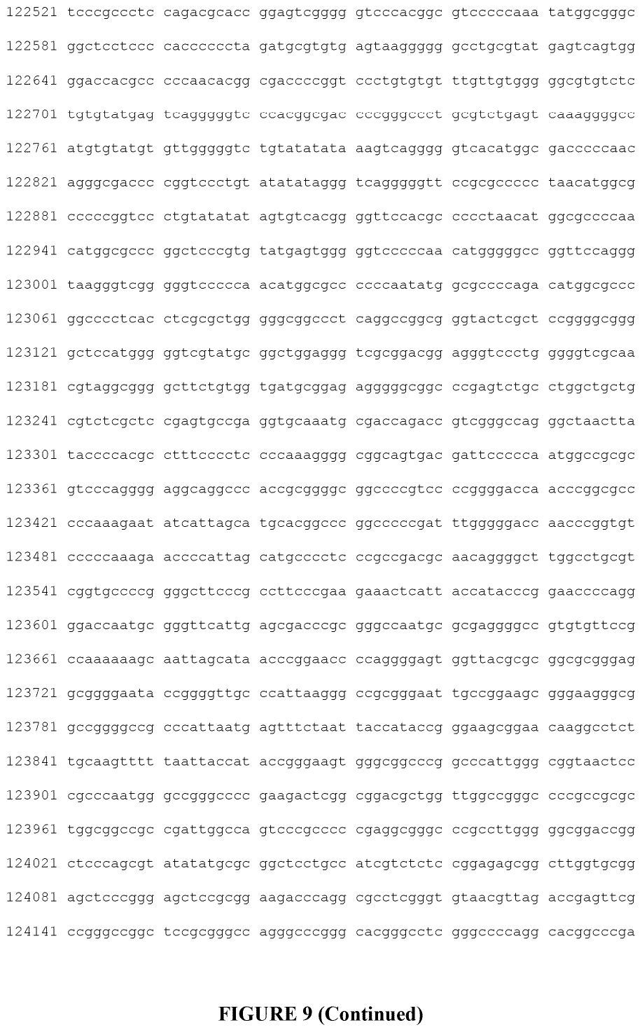

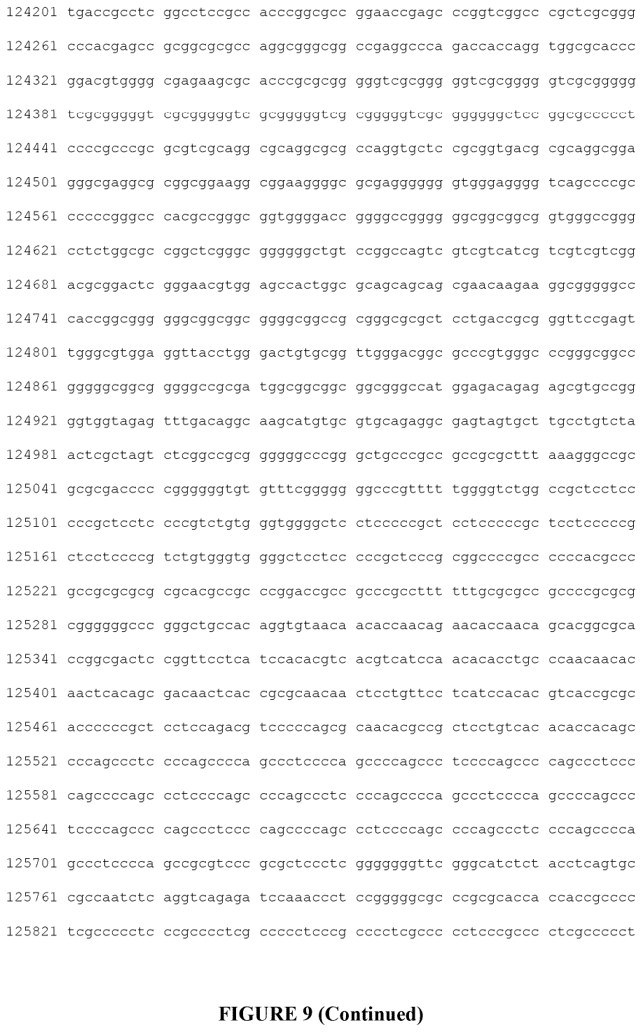

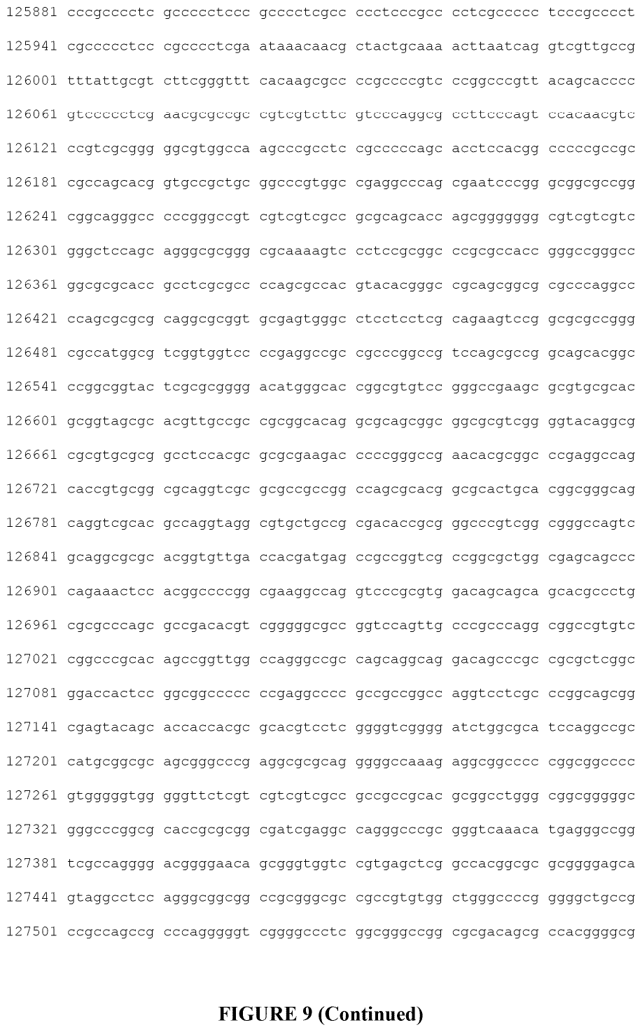

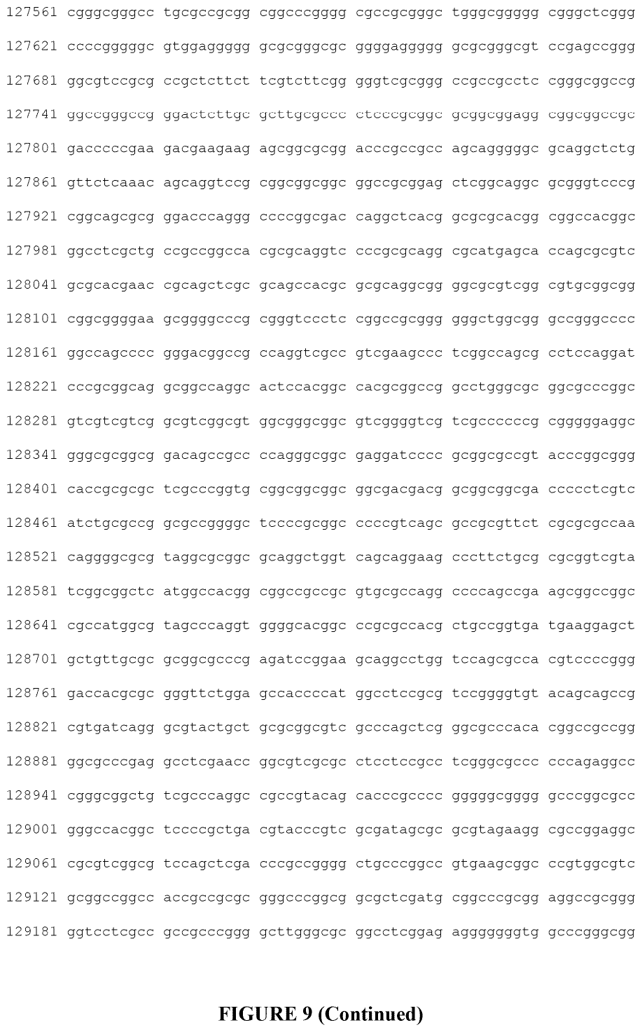

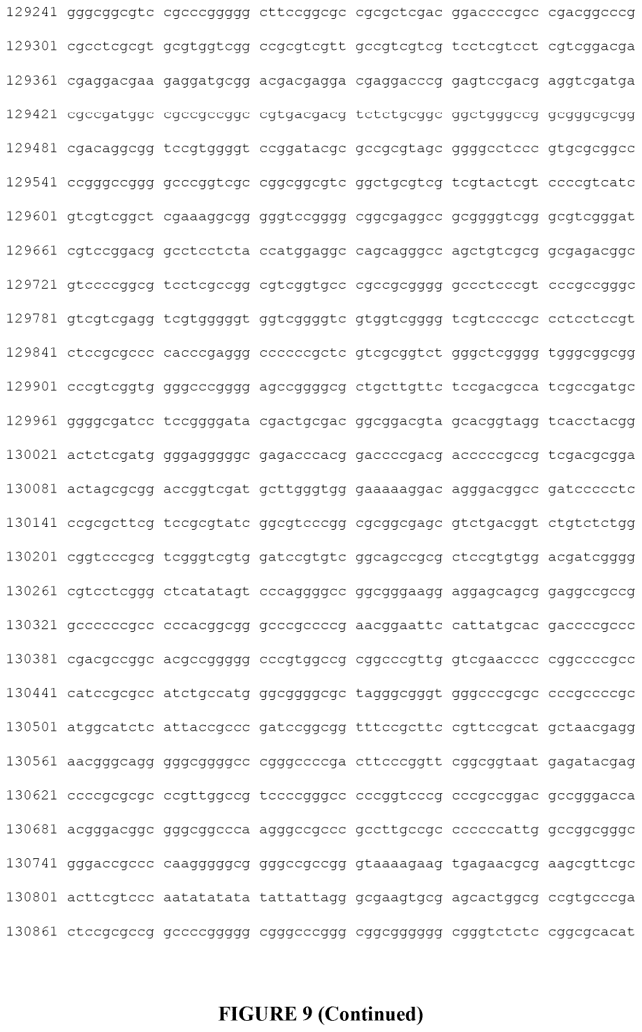

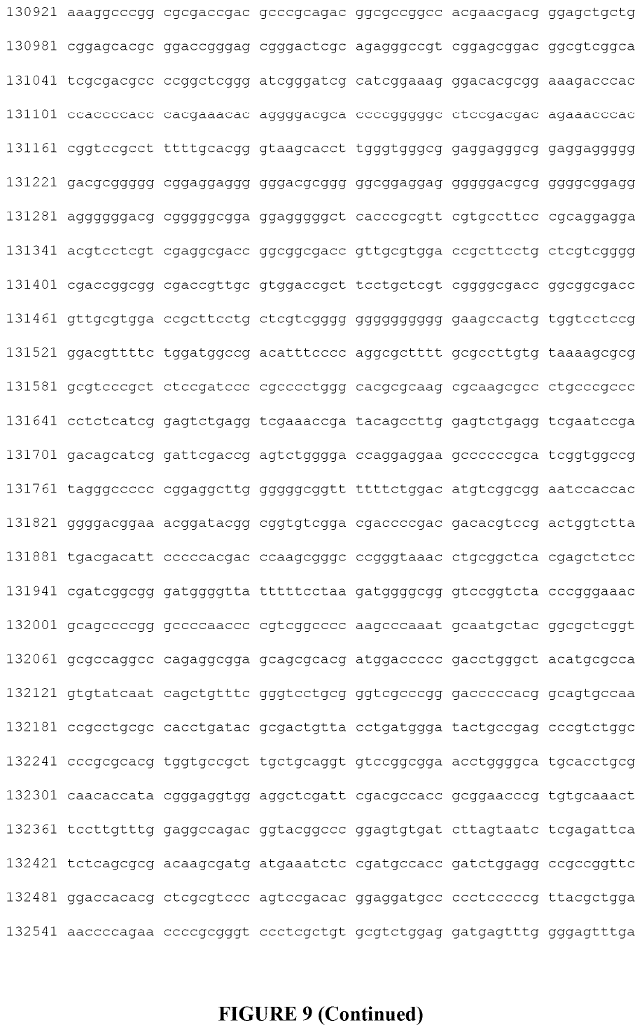

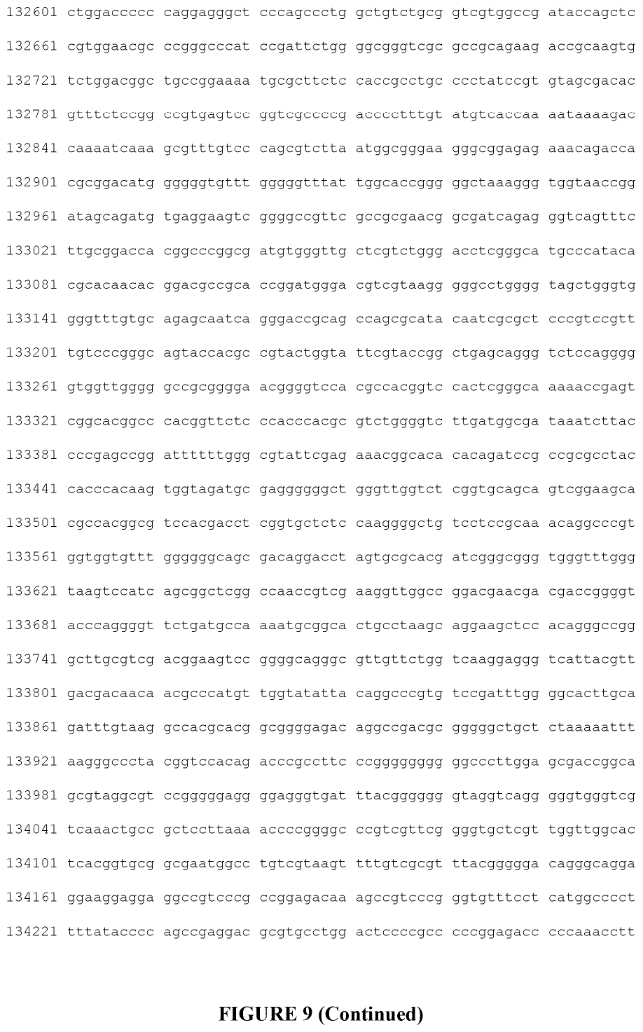

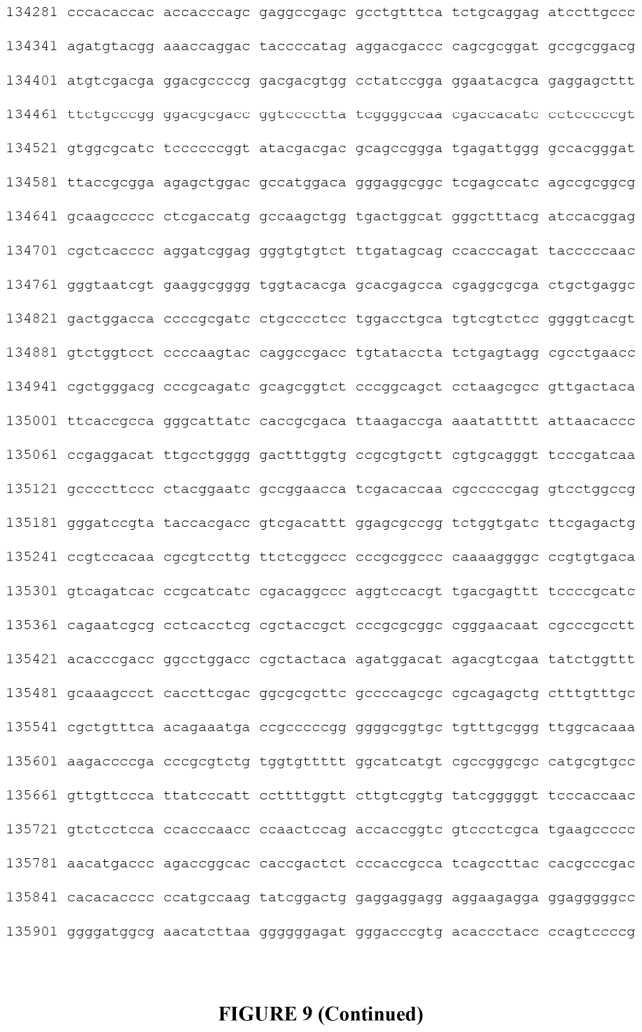

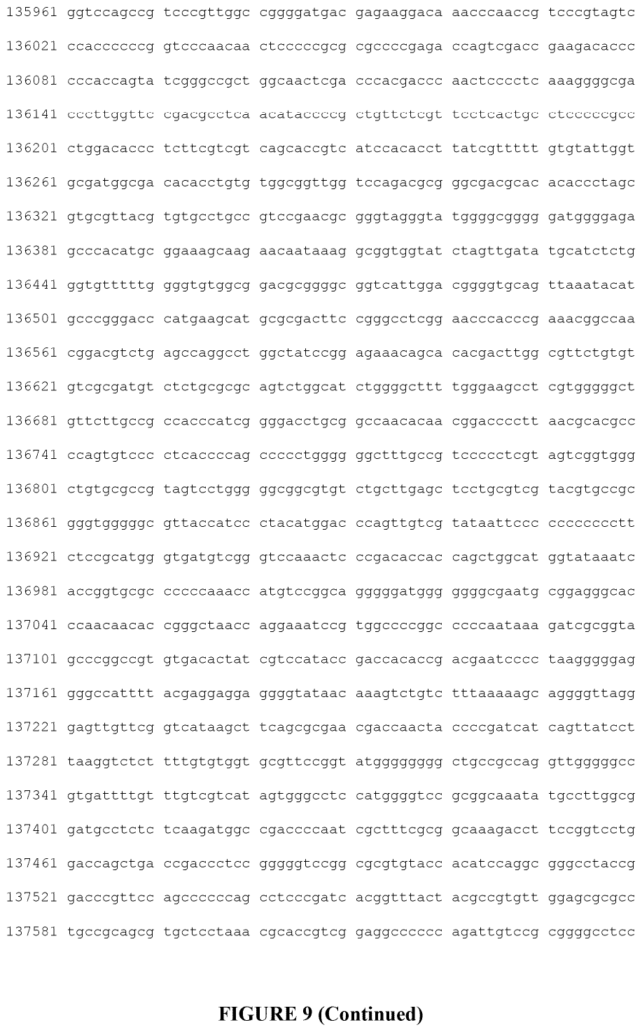

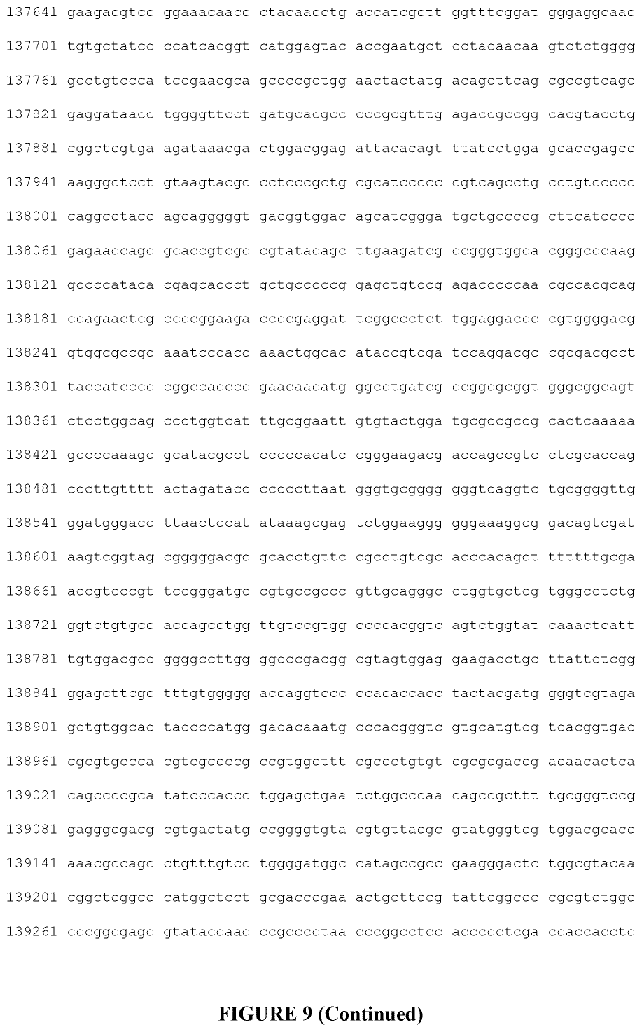

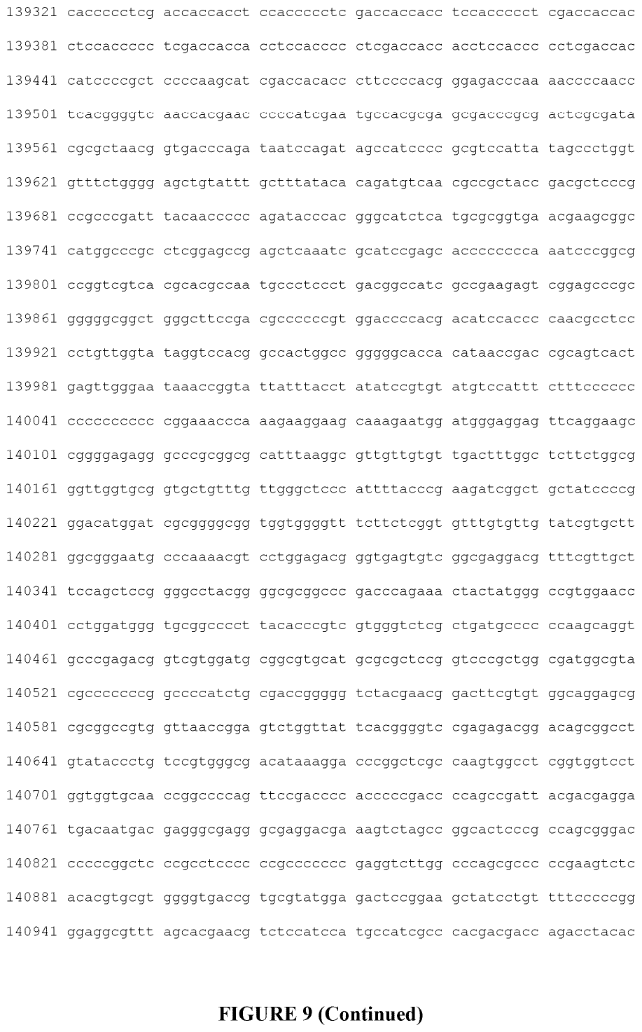

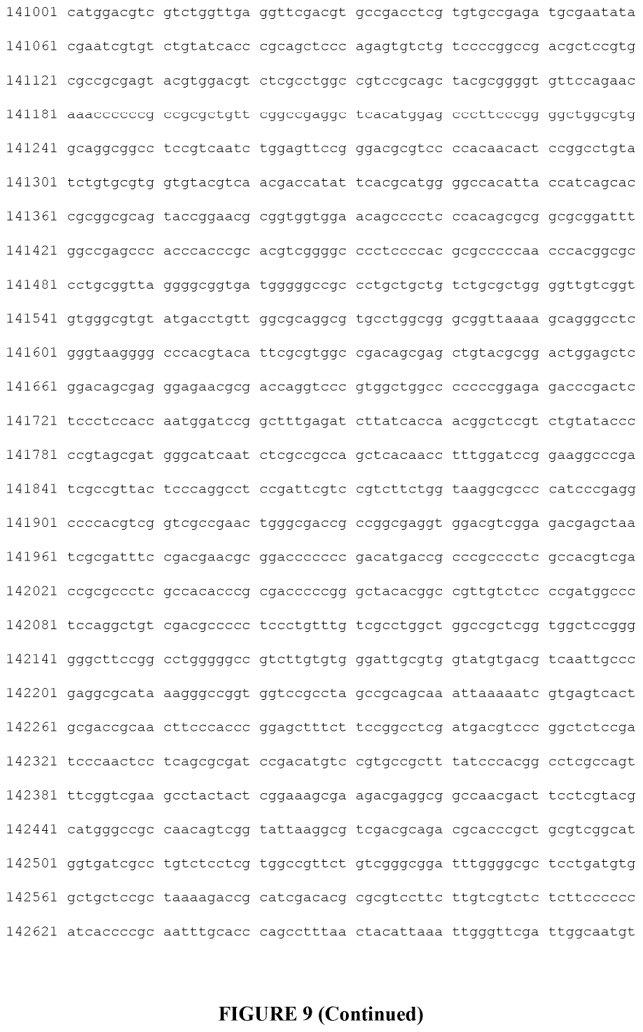

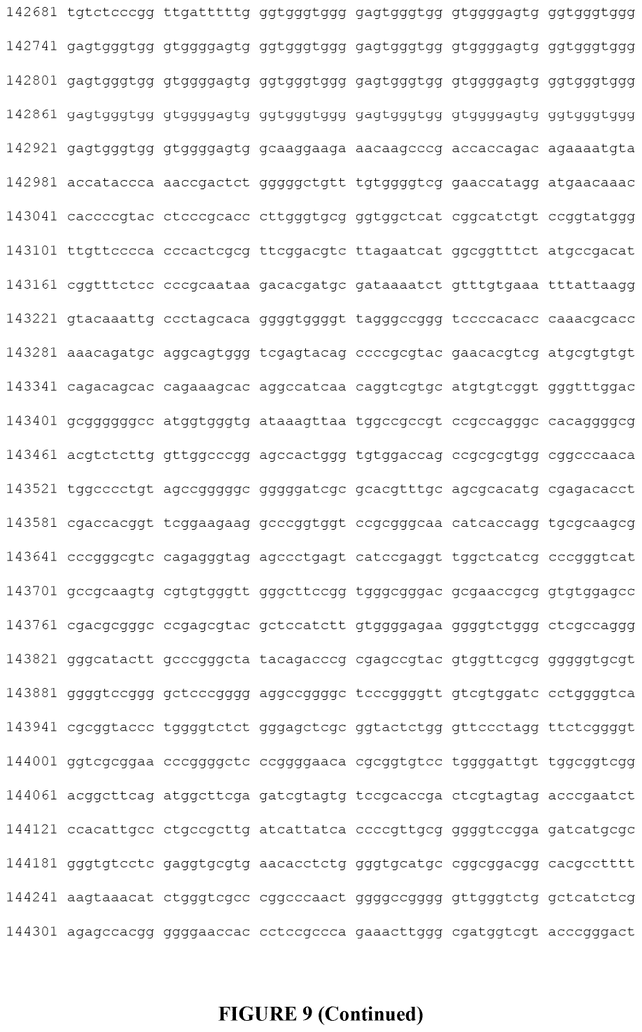

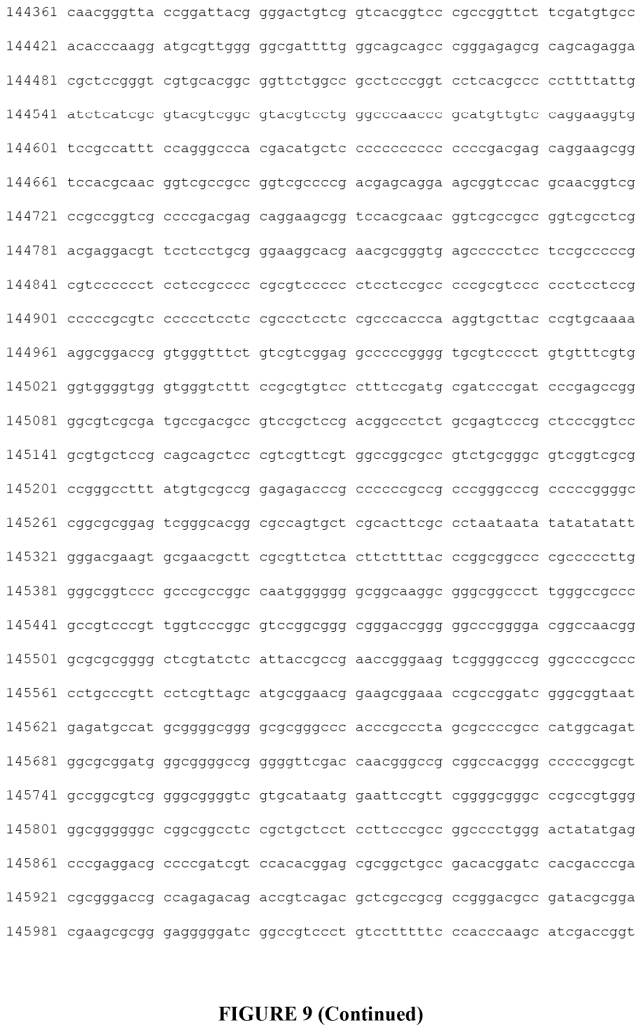

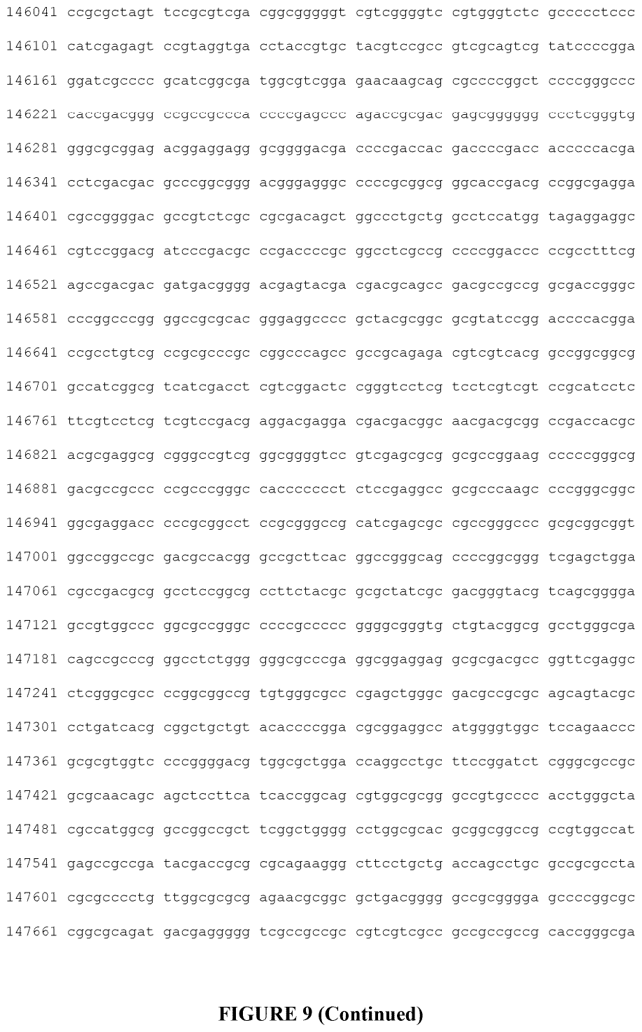

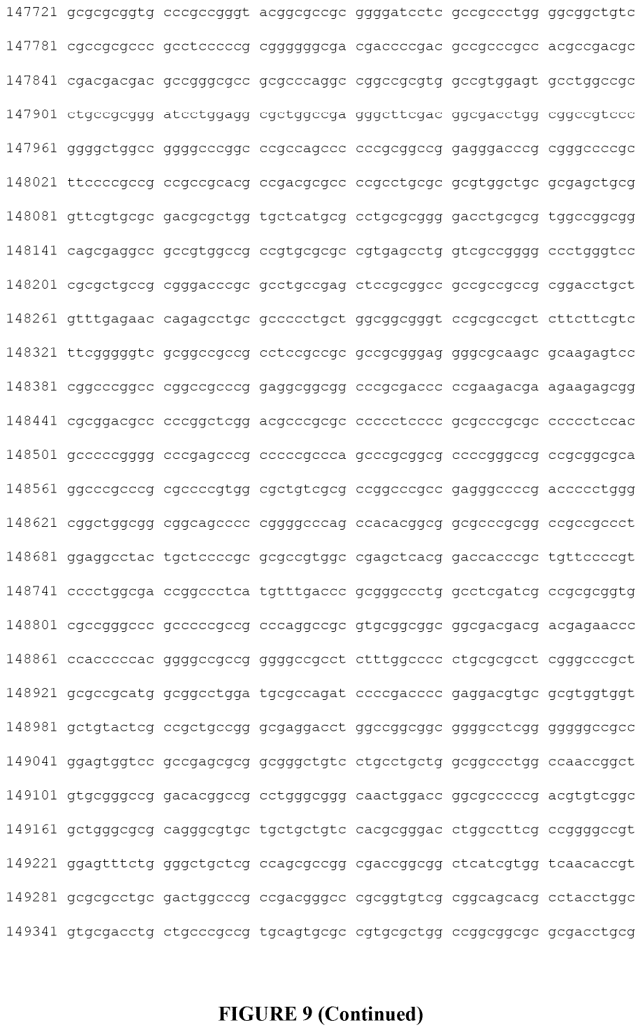

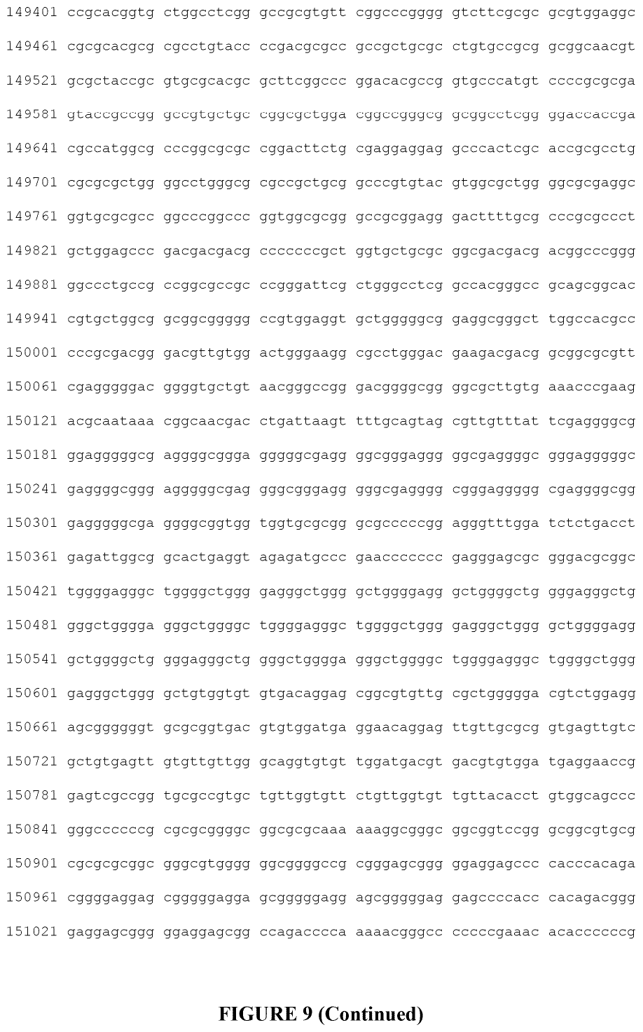

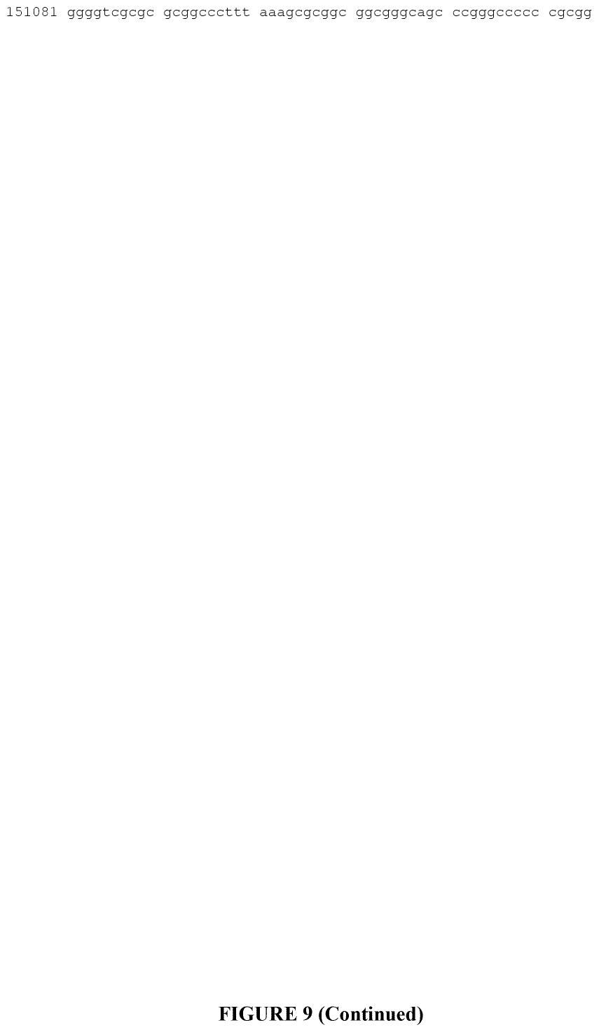

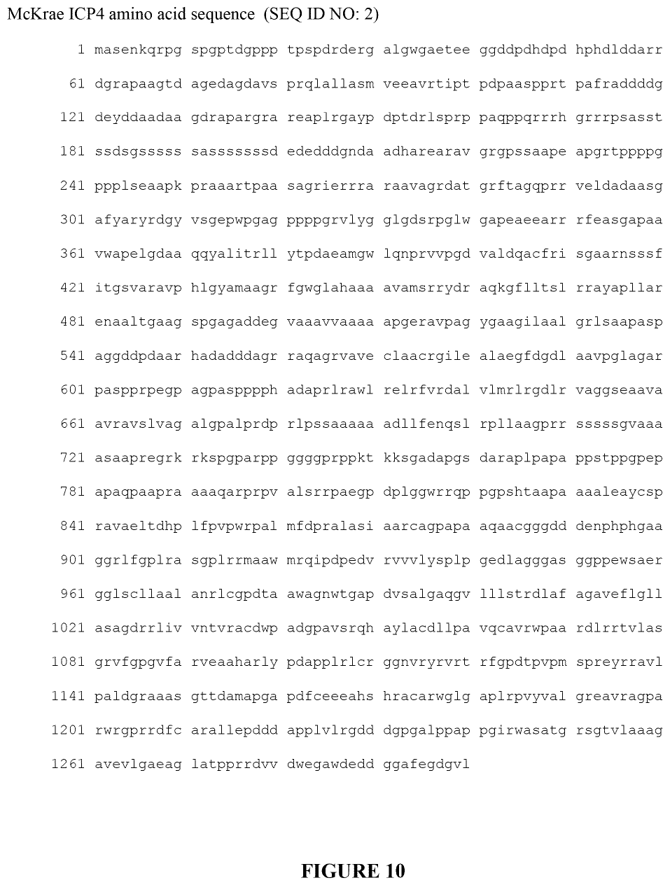

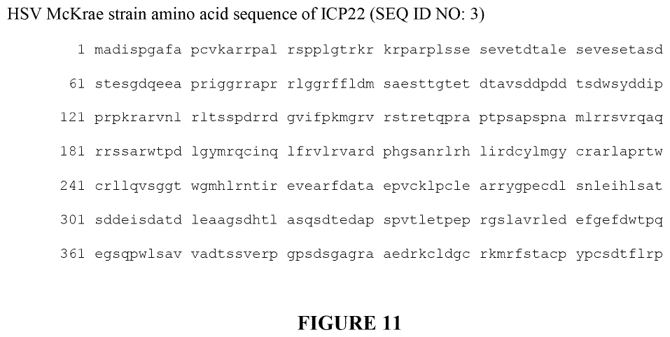

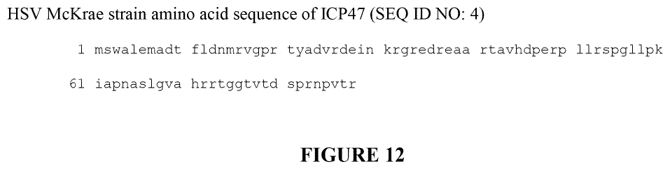

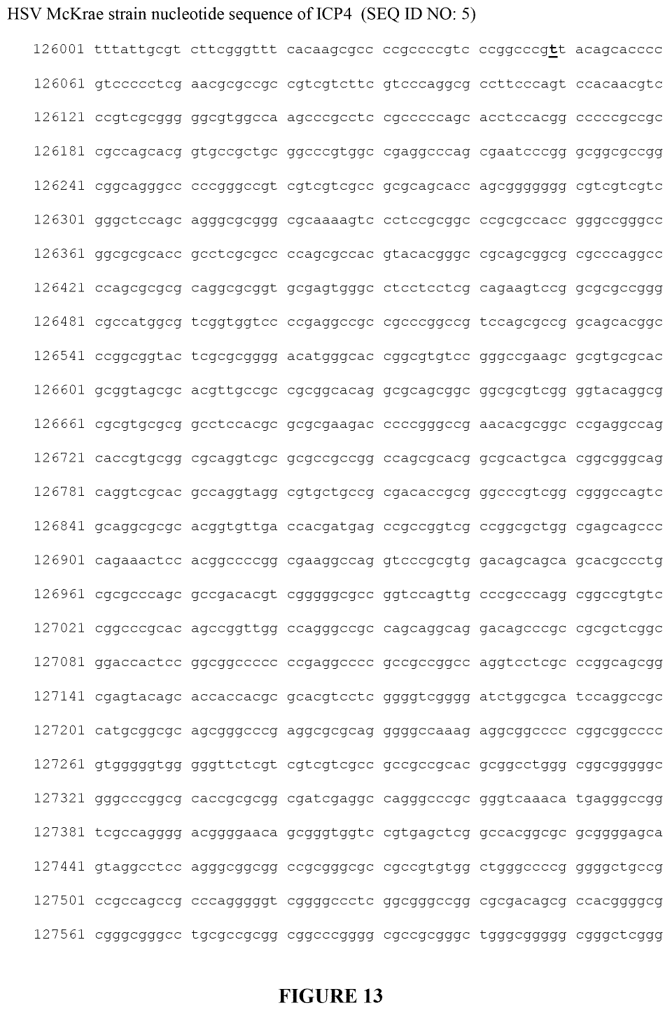

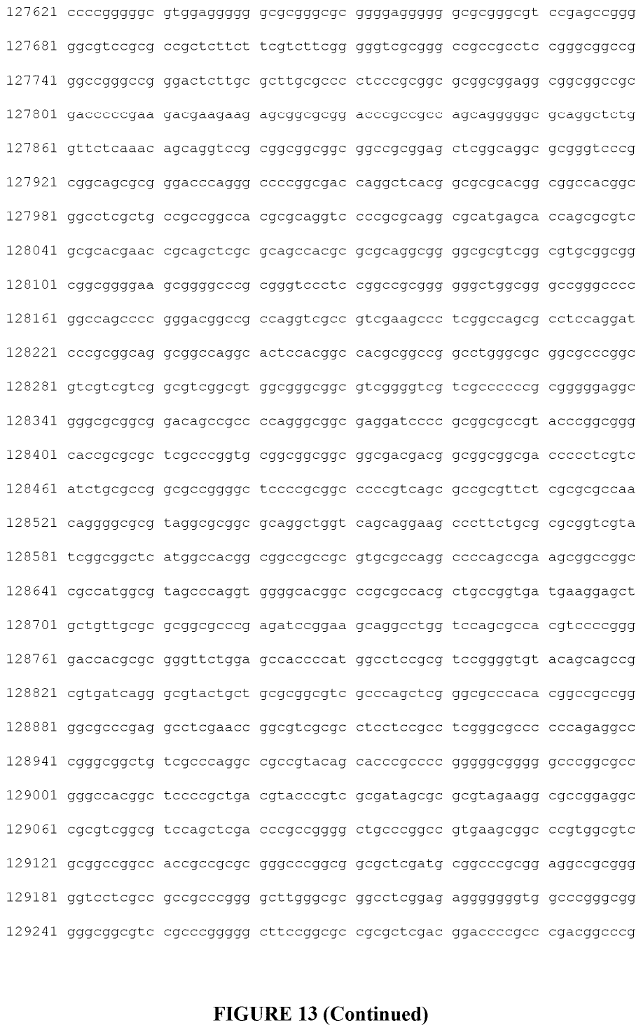

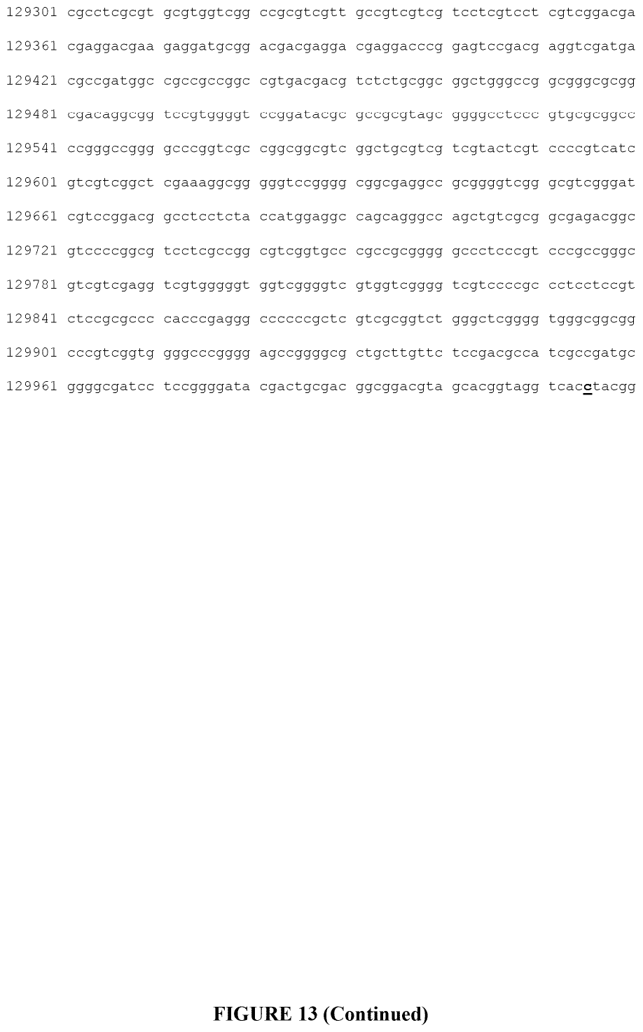

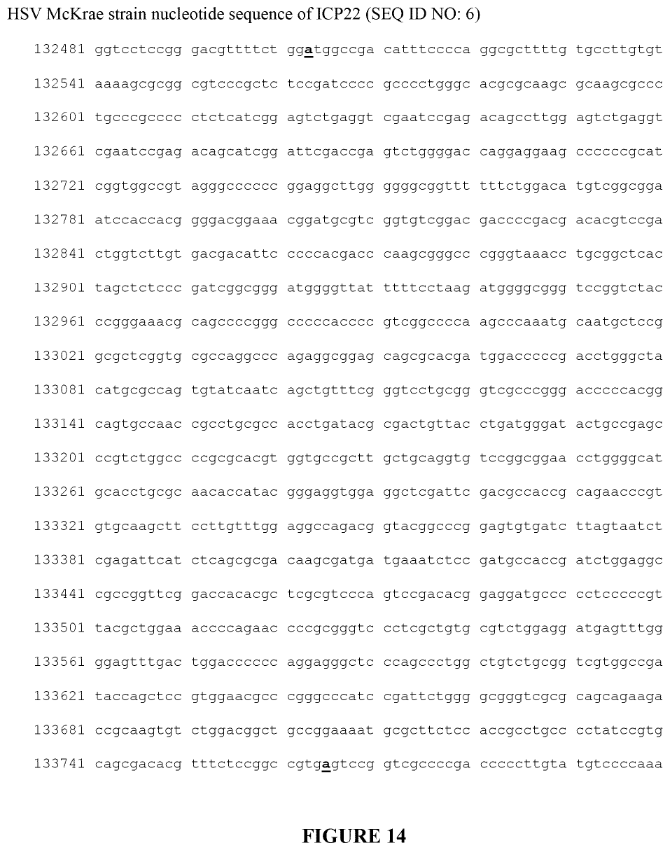

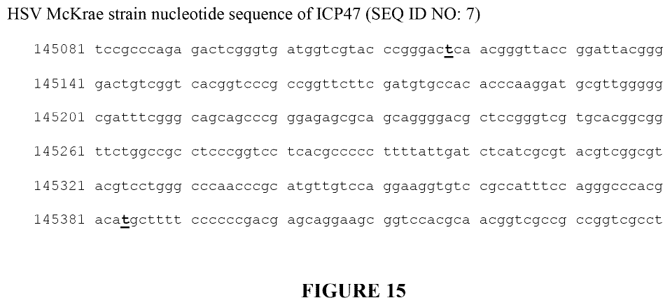

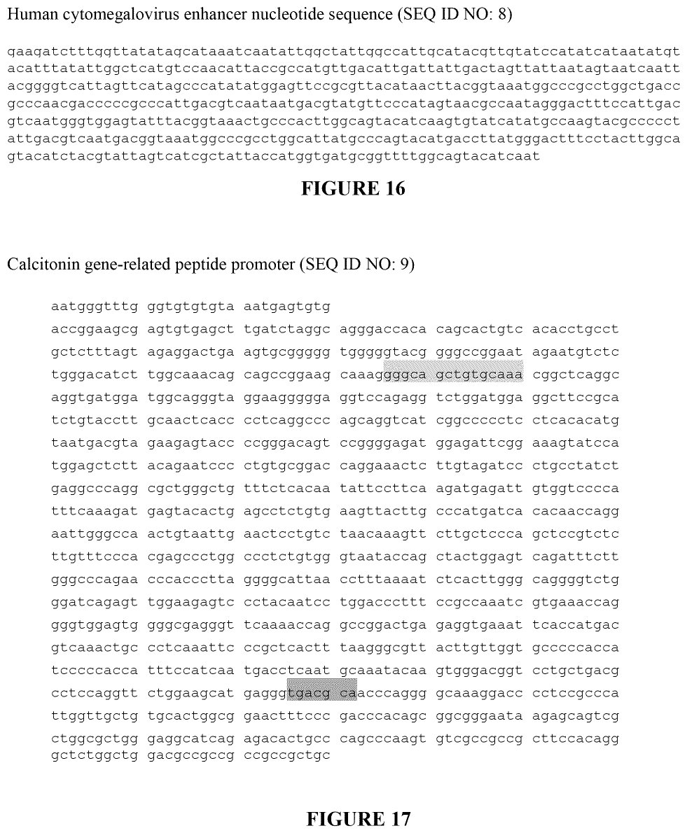

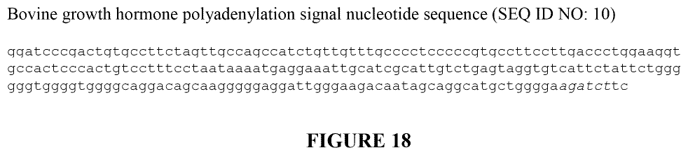

At least 17 strains of HSV-1 have been isolated, including but not limited to, McKrae, strain 17, strain F, H129, HF10, MacIntyre, Strain HF, ATCC 2011 and KOS (for review, see Watson et al, Virology (2012)). A McKrae strain was isolated from a patient with herpes simplex keratitis and subsequently passaged in tissue culture. A partial genome sequence of McKrae is shown in FIG. 9 (SEQ ID NO: 1) (accession number JQ730035).

Inter-strain differences in HSV-1 peripheral replication and virulence are observed after injection into animals. McKrae undergoes spontaneous or induced reactivation at a higher frequency than other known strains and is among the most virulent HSV-1 strains. McKrae is also more neuroinvasive than other known strains, such as strain 17, KOS, F, and H129. In one study, KOS or McKrae was injected into the cornea and genital tract of mice to compare pathogenesis (Wang et al. (2013) Virus Res. 173(2):436-440. Each was found to replicate to a similar extent in the corneal epithelium and trigeminal ganglia; however, McKrae titers were over 100 fold higher in brainstem. Upon intravaginal injection, McKrae and KOS replicated to a similar extent except for a transient spike in McKrae titer at four days. McKrae, but not KOS, elicited significant inflammation of external genitalia along with weight loss in the animals. KOS was not detected in neural tissue and McKrae was rarely detected.

In some embodiments, the disclosure provides HSV viral vectors with deletion of genes that render HSV replication defective, but do not reduce HSV neuroinvasiveness. Thus, the HSV vectors are able to traverse the peripheral nervous system to reach neurons in the dorsal root ganglion upon administration to the skin.

HSV genes influence viral characteristics and phenotype. There are at least 9 genes and several non-coding sequences unique to McKrae strain. In addition to those associated with pathogenesis and latency reactivations, such as RL1, RS1, and RL2, three UL genes (UL36, UL49A, UL56) and three US genes (US7, US10, and US11) are unique for McKrae strain. In addition to gene variations, non-coding sequences such as LAT, ‘a’ sequence, and miRNAs contain variations unique to McKrae.

One or more of following gene and non-coding sequences can be considered characteristic of McKrae strain. In McKrae, RL1 (ICP34.5) has an extended P-A-T repeat between residues 159 and 160 that results in 8 iterations, while other strains contain only 3-5 iterations. The P-A-T repeat is thought to influence cellular localization of the ICP34.5 protein. (Mao & Rosenthal, J. Biol. Chem. 277(13):11423-31 (2012). ICP34.5 is thought to be a neurovirulence factor involved in viral replication and anti-host response.

McKrae strain also contains an extended repeat element of six iterations of the internal tandem repeat STPSTTT (SEQ ID NO: 11) located within the coding sequence of US07 (gI). Additionally in McKrae, UL 36 contains a premature stop codon introduced due to a G nucleotide deletion in a mononucleotide string encoding amino acid residue 2453 (nt 72,535) and UL 56 (180 aa) contains a single base pair insertion at nucleotide 115,992 (amino acid 97). McKrae strain also contains an extended ORF in US10 resulting from a single bp insertion at nucleotide 143,416 and the frameshift causes a stop codon loss in McKrae and a unique C-terminal protein sequence. McKrae has amino acid differences at UL49A at residues 28 and 51 compared to other strains. McKrae has histidine and threonine at residues 28 and 51, respectively, whereas strain 17 has arginine and threonine and other strains (e.g., KOS) have histidine and alanine. Also, McKrae strain contains reduced tandem repeats found at the UL-RL junction (49 bp in McKrae as opposed to 181 bp in strain 17 and KOS) and approximately 330 nucleotides missing immediately following the UL-RL junction repeat. McKrae also contains unique variation within the ‘a’ sequence direct repeat 2 (DR2) array. Instead of a series of unbroken tandem repeats, the McKrae DR2 repeats are interrupted twice by identical guanine-rich sequences.

Major variation within the LAT intron between strains is due to differences in a repeat element (GCACCCCCACTCCCAC) (SEQ ID NO: 12) that varies in iteration number beginning at nucleotide 119,482 in McKrae strain, with McKrae containing 13 repeats while strains F, H129 and 17 contain 9 repeats and KOS contains 15 repeats. Also, tandem repeat variation between strains is found beginning in McKrae at base 125,520. McKrae repeat elements include twelve iterations of CCCCAGCCCTCCCCAG (SEQ ID NO: 13) and eight iterations of CCCCTCGCCCCCTCCCG (SEQ ID NO: 14). The first repeat unit is unique from other strains in that it contains a G-A transition, and strain McKrae contains three iterations more than any other strain. The McKrae strain second repeat element is collapsed, missing 188 nucleotides relative to all other strains, and separated from the upstream repeat by a 100% conserved sequence of 105 bp containing miR-H5.

McKrae further contains a unique coding sequence for ICP4 that is not found in other known strains. (Watson et al., Virology (2012)). ICP4 is an immediate early transcriptional regulator and has been implicated in reactivation. Whereas other strains contain an alanine rich region (AASAPDAADALAAA) (SEQ ID NO: 15) between residues 707 and 720, in McKrae the alanine rich region is replaced by a serine rich sequence (GPRRSSSSSGVAA) (SEQ ID NO: 16). The serine rich block of substitutions present in McKrae is adjacent to the nuclear localization signal (NLS) (amino acid 728-734). A change in conformation of this region may alter the NLS and in turn affect localization of not only ICP4, but also other viral proteins (e.g. ICP0, ICP8) that are affected by ICP4 localization (Knipe and Smith, 1986). Thus, this region may influence viral phenotype in part by altering the localization of proteins to the nucleus.

### Replication Defective McKrae Vector

McKrae Backbone

Viral genes are expressed in a tightly regulated, ordered cascade, which begins with the production of the immediate-early (IE) genes. The resulting IE proteins, which include infected cell proteins ICP0, ICP4, ICP22, ICP27, and ICP47, are responsible for regulating viral gene expression during subsequent phases of the replication cycle. Replication-defective variant viruses are defective for one or more functions that are essential for viral genome replication or synthesis and assembly of viral particles. Such viruses can be propagated in complementing cell lines expressing the missing gene product(s); however, in normal (i.e., non-complementing) cells, the viruses express viral gene products but do not replicate to form progeny virions.

Replication-defective viruses can be created through various methods known in the art for modifying genes. In some embodiments, one or more nucleotides are rendered different relative to the wild-type sequence. In some embodiments, one or more nucleotides are deleted. In some embodiments, the deletion of one or more nucleotides creates a premature stop codon. In some embodiments, the deletion of one or more nucleotides creates a gene encoding a truncated polypeptide. In some embodiments, the deletion of one or more nucleotides creates a gene encoding a nonfunctional polypeptide. In some embodiments, the deletion of one or more nucleotides renders a gene nonfunctional by disruption. In some embodiments, a gene is disrupted by deletion of its promoter.

In some embodiments, one or more genes are deleted to render a virus replication defective. In some embodiments, the gene encoding ICP0 is fully or partially deleted. In some embodiments, the gene encoding ICP4 is fully or partially deleted. In some embodiments, the gene encoding IC22 is fully or partially deleted. In some embodiments, the gene encoding ICP27 is fully or partially deleted. In some embodiments, the gene encoding ICP47 is fully or partially deleted. In some embodiments, the gene encoding ICP 4 is fully or partially deleted, without disrupting expression of any additional immediate early genes. In some embodiments, the gene encoding ICP4 is fully or partially deleted, and one or more other immediate early (IE) genes are disrupted. In some embodiments, the gene encoding ICP4 is deleted and ICP22 and ICP47 are disrupted.

HSV-1 IE promoters contain one or more copies of an IE-specific regulatory sequence of consensus TAATGARAT (SEQ ID NO: 19) (where R is a purine). These motifs are normally located within a few hundred base pairs of the proximal IE promoter sequences, but in conjunction with their flanking sequences they are discrete functional entities which can confer IE-specific regulation to other proximal promoter elements of different temporal class. In some embodiments, replication-defective viruses are created by deleting nucleotides in an IE-specific regulatory sequence. In some embodiments, an IE-specific regulatory sequence contains an internal deletion. In some embodiments, an IE-specific regulatory sequence contains a terminal deletion. In some embodiments, an IE-specific regulatory sequence is completely deleted.

A schematic of an exemplary replication defective McKrae strain viral vector is depicted below. The schematic shows complete deletions of both copies of the viral ICP4 gene, and a human cytomegalovirus (HCMV) immediate early promoter driven expression cassette inserted within both copies of the deleted ICP4 loci. The expression cassette contains a payload of interest for expression in target cells.

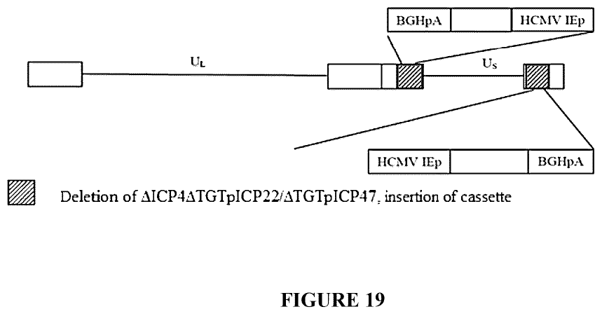

The extent of the ICP4 deletion results in the removal of the upstream promoter sequences of two additional immediate early viral genes: ICP22 and ICP47 (FIG. 19).

Payload

Viral vectors in accordance with the present disclosure contain a nucleic acid molecule comprising the payload of the vector. In some embodiments, a payload comprises a nucleic acid molecule that encodes a protein. In some embodiments, a payload comprises a nucleic acid molecule that comprises a sequence complementary to a nucleic acid sequence that encodes a protein. In some embodiments, a payload encodes a nucleic acid molecule that is regulatory. In some embodiments, a payload encodes a small interfering RNA (siRNA) polynucleotide. In some embodiments, a payload encodes a micro RNA (miRNA) polynucleotide.

In some embodiments, the payload is a nucleic acid molecule that encodes a protein that is exogenous to the target tissue or subject to which the vector is administered. In some embodiments, the payload is a nucleic acid molecule that encodes a protein that is endogenous to the target tissue or subject to which the vector is administered. In some embodiments, a nucleic acid molecule is codon optimized.

Regulatory Elements

The inclusion of non-native regulatory sequences, gene control sequences, promoters, non-coding sequences, introns, or coding sequences in a nucleic acid of the present disclosure is contemplated herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid of the present disclosure can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. Promoters having stochastic expression, inducible expression, conditional expression, or otherwise discontinuous, inconstant, or unpredictable expression are also included within the scope of the present disclosure. A promoter of the present disclosure may include any of the above characteristics or other promoter characteristics known in the art.

Examples of known promoters include, but are not limited to, the cytomegalovirus (CMV) promoter CMV/human beta 3 globin promoter GFAP promoter, chicken beta actin (CBA) promoter the β-glucuronidase (GUSB) promoter and ubiquitin promoters such as those isolated from human ubiquitin A, human ubiquitin B, and human ubiquitin C.

In some embodiments, a promoter is a neuron specific promoter in that it is a promoter having specific expression in neurons, preferential expression in neurons, or that typically drives expression of an associated coding sequence in neurons or a subset of neurons but not in one or more other tissues or cell types. Examples of such promoters include calcitonin gene-related peptide (CGRP), synapsin I (SYN), calcium/calmodulin-dependent protein kinase II, tubulin alpha I, neuron-specific enolase, microtubule-associated protein IB (MAPI B), and platelet-derived growth factor beta chain promoters, as well as derivatives thereof. In some embodiments, the promoter is a calcitonin gene-related peptide (CGRP) promoter or derivative thereof.

Other regulatory elements may additionally be operatively linked to the payload, such as an enhancer and a polyadenylation site. In some embodiments, an enhancer comprises a human cytomegalovirus (HCMV) sequence. In some embodiments, a polyadenylation site comprises a bovine growth hormone (BGH) polyadenylation signal.

In some embodiments, a promoter is a chimeric of one or more promoters or regulatory elements found in nature. In some embodiments, the viral vectors comprise a payload whose expression is driven by a CGRP promoter with an HCMV enhancer sequence.

### Preparation of Vectors

The present disclosure relates particularly to McKrae strain viral vectors that are replication defective. In some embodiments, viral vectors are generated by deletion or disruption of one or more immediate early genes. Viral genes may be deleted or disrupted using methods of recombinant technology known in the art. In some embodiments a viral vector of the present disclosure may be rendered replication defective as a result of a homologous recombination event. In some embodiments, replication defective viral vectors are generated by deletion of an ICP4 gene. In some embodiments, replication defective viral vectors are generated by deletion of an ICP4 gene and deletion of a promoter for one or more other immediate early genes (e.g., ICP22 and/or ICP47).

In some embodiments, viral vectors of the present disclosure are generated by deletion of loci encoding one or more ICPs (e.g., ICP4) through homologous recombination. In some embodiments, generation of a viral vector of the present disclosure includes a step of homologous recombination of a first plasmid with a second plasmid. In some embodiments, the first plasmid contains nucleic acid sequences homologous to regions of an HSV genome that are adjacent to a nucleic acid region of an HSV genome that is intended to be replaced. In some embodiments, the second plasmid contains an HSV genome, or fragment thereof. In some embodiments, the first plasmid contains nucleic acid sequence encoding a gene of interest between the homologous nucleic acid sequences. In some embodiments, the gene of interest may be or include a marker protein that is detectable by fluorescence, chemiluminescense, or other property, which can be used to select for vectors resulting from successful homologous recombination.

In some embodiments, a viral vector of the present disclosure is generated by homologous recombination of a first plasmid containing a nucleic acid sequence homologous to regions upstream of the ICP4 promoter including the viral origin contained within the short inverted repeat regions of HSV, with a second plasmid containing an HSV McKrae strain genome.

In some embodiments, a vector is made by first replacing both copies of the ICP4 loci by homologous recombination using plasmid SASB3 and screening for green fluorescent protein (GFP)-expressing plaques. In some embodiments, a plasmid is constructed by cloning the Sph I to Afl III (Sal I linkered) fragment (1928 bp) of the HSV-1 KOS strain genome (nucleotides 124485-126413) into Sph I/Sal I digested pSP72 followed by insertion of the 695 bp Bgl II to BamH I fragment (nucleotides 131931 to 132626) containing regions upstream of the ICP4 promoter including the viral origin contained within the short inverted repeat regions into the Bgl 11 to BamH I sites of the vector plasmid. In some embodiments, a plasmid is constructed by cloning a HCMV-eGFP fragment in the BamHI site of a plasmid as described above. In some embodiments, a plasmid as described above is then recombined into a specific locus of a wild-type McKrae virus. In some embodiments, the resulting vector is isolated using a stable cell line that expresses one or more genes deleted or disrupted in the HSV genome that are required for replication.

In some embodiments, a vector is made by first replacing both copies of the ICP4 loci by homologous recombination using plasmid SDAXB and screening for green fluorescent protein (GFP)-expressing plaques. In some embodiments, a plasmid is constructed by cloning the Sph I to Afl III fragment (1928 bp) of the HSV-1 KOS strain genome (nucleotides 124346 to 126273 of accession KT899744) into Sph I/Afl III digested pSP72 to make SDA followed by changing the Afl III site to a BamHI site (SDAB). A BamHI to Bgl II DNA PCR fragment containing regions upstream of the ICP4 promoter including the viral origin (nucleotides 144933 to 145534 of accession JQ730035) contained within the short inverted repeat regions was cloned into the BamHI site of SDAB to make SDAXB. In some embodiments, a plasmid is constructed by cloning a HCMV-eGFP fragment in the BamHI site of a plasmid as described above. In some embodiments, a plasmid as described above is then recombined into a specific locus of a wild-type McKrae virus. In some embodiments, the resulting vector is isolated using a stable cell line that expresses one or more genes deleted or disrupted in the HSV genome that are required for replication.

### Characterization of Vectors

Viral vectors in accordance with the present disclosure can be characterized by genomic sequencing in order to determine if the expected vector was successfully created. Any method of sequencing known in the art is acceptable for this purpose. Methods of sequencing include, for example, nanopore sequencing, single molecule real time sequencing (SMRT), DNA nanoball (DNB) sequencing, pyrosequencing and using DNA arrays.

The expression of a payload from a viral vector can be detected by any method known in the art for detecting proteins or nucleic acids. Methods of detecting protein expression include immunohistochemistry, flow cytometry, Western blotting, enzyme-linked immunosorbent assay (ELISA), immune-electron microscopy, individual protein immunoprecipitation (IP), protein complex immunoprecipitation (Co—IP), chromatin immunoprecipitation (ChIP), RNA immunoprecipitation (RIP), immunoelectrophoresis, spectrophotometry, and bicinchoninic acid assay (BCA). Methods of detecting nucleic acid expression include Southern blotting, Northern blotting, polymerase chain reaction (PCR), quantitative PCR, and RT-PCR.

In some embodiments, the present disclosure provides methods for testing the ability of viral vectors to transduce neurons. In some embodiments, the neurons are peripheral neurons. In some embodiments, the neurons are sensory neurons. In some embodiments, the neurons comprise dorsal root ganglia (DRG).

In some embodiments, a viral vector preparation may be injected into the one or more dermatomes corresponding to a section of DRG for example, the left and right L4, L5, and L6 DRG. DRG are removed are removed and DNA is isolated from the DRG and analyzed for vector genome copies using a qPCR assay that targets a sequence within HSV-1. In some embodiments, a qPCR assay targets a sequence within the HSV-1 glycoprotein (U_{L}-22) gene

### Applications/Uses

Viral vectors in accordance with the present disclosure are useful for a wide variety of therapeutic applications. In some embodiments, vectors as described herein are useful to deliver one or more payloads to one or more target cells. In some embodiments, target cells reside in tissues that are poorly vascularized and difficult to reach by systemic circulation. In some embodiments, target cells are cells susceptible to infection by HSV. In some embodiments, target cells are particularly susceptible to infection by a McKrae strain of HSV. In some embodiments, target cells are or include one or more of neuronal cells. In some embodiments, target cells are dorsal root ganglion (DRG) cells.

Gene Therapy

Viral vectors in accordance with the present disclosure are useful in any context in which gene therapy is contemplated. For example, viral vectors comprising a heterologous nucleic acid segment operably linked to a promoter are useful for any disease or clinical condition associated with reduction or absence of the protein encoded by the heterologous nucleic acid segment, or any disease or clinical condition that can be effectively treated by expression of the encoded protein within the subject. Viral vectors that contain an expression cassette for synthesis of an RNAi agent (e.g., one or more siRNAs or shRNAs) are useful in treating any disease or clinical condition associated with overexpression of a transcript or its encoded protein in a subject, or any disease or clinical condition that may be treated by causing reduction of a transcript or its encoded protein in a subject. Viral vectors that comprise an expression cassette for synthesis of one or more RNAs that self-hybridize or hybridize with each other to form an RNAi agent targeted to a transcript encoding a cytokine may be used to regulate immune system responses (e.g., responses responsible for organ transplant rejection, allergy, autoimmune diseases, inflammation, etc.). Viral vectors that provide a template for synthesis of one or more RNAs that self-hybridize or hybridize with each other to form an RNAi agent targeted to a transcript of an infectious agent or targeted to a cellular transcript whose encoded product is necessary for or contributes to any aspect of the infectious process may be used in the treatment of infectious diseases.

Administration

Compositions comprising viral vectors as described herein may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include intradermal. In some embodiments, pharmaceutical compositions include a viral vector in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. In some embodiments, viral vectors are formulated in glycerol. In some embodiments, viral vectors are formulated in approximately 10% glycerol in phosphate buffered saline.

It is advantageous to formulate compositions in dosage unit form for ease of administration and uniformity of dosage. Dosage unit form as used herein refers to physically discrete units suited as unitary dosages for the subject to be treated; each unit containing a predetermined quantity of a viral vector calculated to produce the desired therapeutic effect in association with a pharmaceutical carrier.

The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a viral vector can include a single treatment or, in many cases, can include a series of treatments.

Compositions

In some embodiments, the active agents, i.e., a viral vector of the disclosure and/or other agents to be administered together with a viral vector of the disclosure, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. In some embodiments the composition is targeted to particular cell types or to cells that are infected by a virus.

Combination Therapy

According to the present disclosure, provided compositions may be administered in combination with one or more other active agents and/or therapeutic modalities, such as known therapeutic agents and/or independently active biologically active agents. In some embodiments, provided compositions include one or more such other active agents; in some embodiments, such other active agents are provided as part of distinct compositions. In some embodiments, combination therapy involves simultaneous administration of one or more doses or units of two or more different active agents and/or therapeutic modalities; in some embodiments, combination therapy involves simultaneous exposure to two or more different active agents and/or therapeutic modalities, for example through overlapping dosing regimens.

In some embodiments, provided compositions include or are administered in combination with one or more other active agents useful for the treatment of the relevant disease, disorder and/or condition.

### EXAMPLES

### Example 1: Assay for Assessment of Transduction of DRG

This Example shows an exemplary method for assaying transduction of viral vectors in dorsal root ganglion (DRG) tissue.

Subsequent to intradermal administration of a viral vector, L4, L5, and L6 DRG are removed and vortexed, with inversion, for 40 seconds in a pre-chilled Lysing Matrix A tube (MP Biomedicals) with 350 μL of a 0.5% Reagent DX (Qiagen) in Buffer RLT Plus/DTT

### Solution

DNA and RNA are isolated from the sample homogenate using the AllPrep DNA/RNA Mini Kit (Qiagen). The RNA isolation portion includes an on-column DNase treatment step. The DNA is eluted in 2×190 μL of UltraPure Distilled Water (Invitrogen) after a 10-15 minute room temperature incubation per elution. The RNA is eluted in 2×30 μL of RNase-free water* after a 3 minute room temperature incubation per elution. The DNA is concentrated by open incubation at 37° C. overnight.

Ten (10) μL of concentrated DNA is analyzed in a 50-μL reaction for HSV vector genomes by a qPCR assay that targets a region in the U_{L}22 (glycoprotein H) gene.

For mRNA expression analyses, 8 μL of RNA first undergoes reverse transcription using the SuperScript™ III First-Strand Synthesis SuperMix for qRT-PCR (Invitrogen) followed by RNase treatment. The RNase-treated cDNA can then be analyzed by any of a number of qPCR assays that target either a particular transgene (e.g., payload transcript), the stable 2kb LAT intron, or the 5′ LAT exon.

### Example 2: Comparison of Nerve Transduction Capabilities of Different HSV Strains

This example demonstrates that a McKrae strain vector transduces neurons more effectively than a KOS strain vector.

Two different wild-type strains of HSV-1 (McKrae and KOS) were prepared and injected into the dorsum and plantar surface of the right and left hind feet of three Sprague-Dawley (SD) rats each, at 100 μL per injection. All animals were euthanized five days after vector injection. During the terminal procedures, the left and right L4, L5, and L6 dorsal root ganglia (DRG) were removed, frozen at −70° C., and shipped on dry ice.

DNA was isolated from the left L4-L6 DRG of all animals in the study using a QIAamp DNA Mini Kit (Qiagen). The sample DNA was analyzed for vector genome copies using a qPCR assay that targets a sequence within the HSV-1 glycoprotein H (UL-22) gene on a Rotor-Gene Q Real-Time PCR Cycler (Qiagen). As shown in Table 1, McKrae strain appears to transduce the neurons significantly better than the KOS strain. The mean genome copy number detected in the DRG of the KOS group was 73, while that of the McKrae group was 21,347.

TABLE 1

| | | | Calc | | Mean | Mean | | Total |
|---|---|---|---|---|---|---|---|---|
| | | | Conc | Mean | Ct | Calc | Total | Sample |
| Strain | Name | Ct | (Copies) | Ct | SD | Conc | DF | Genomes |
| KOS | 5a1R A1G1 L-DRG | 35.27 | 7.00E±00 | 35.31 | 0.06 | 7.00E±00 | 20 | 1.40E±02 |
| | 5a1R A1G1 L DRG | 35.36 | 6.00E±00 | | | | | |
| KOS | 5a1R A2G1 L-DRG | 36.10 | 4.00E±00 | 36.05 | 0.07 | 4.00E±00 | 20 | 8.00E±01 |
| | 5a1R A2G1 L DRG | 36.01 | 4.00E±00 | | | | | |
| KOS | 5a1R A3G1 L-DRG | 37.15 | 2.00E±00 | 37.15 | | 2.00E±00 | 34.97 | <LOQ |
| | 5a1R A3G1 L-DRG | | | | | | | |
| Mck | 5a1R A4G2 L-DRG | 37.31 | 2.00E±00 | 36.43 | 1.25 | 3.00E±00 | 40.65 | <LOQ |
| | 5a1R A4G2 L ORG | 35.54 | 6.00E±00 | | | | | |
| Mck | 5a1R A5G2 L-DRG | 26.73 | 1.95E±03 | 26.74 | 0.01 | 1.94E±03 | 32.89 | 6.38E±04 |
| | 5a1R A5G2 L DRG | 26.74 | 1.93E±03 | | | | | |
| Mck | 5a1R A6G2 L-DRG | 35.23 | 7.00E±00 | 34.79 | 0.61 | 9.00E±00 | 28.57 | 2.57E±02 |
| | 5a1R A6G2 L-DRG | 34.36 | 1.20E±01 | | | | | |

| | | Calc | | Mean | Mean | | |
|---|---|---|---|---|---|---|---|
| | | Conc | Mean | Ct | Calc | Spk | % |
| Name | Ct | (Copies) | Ct | SD | Conc | (Cop) | Recovery |
| 5aS2 A2G1 L-DRG | 36.93 | 2 | 37.22 | 0.42 | 2 | <LOQ | |
| 5aS2 A2G1 L-DRG | 37.52 | 2 | | | | | |
| 5aS2 A2G1 L-DRG spk-25 | 32.67 | 38 | 32.79 | 0.18 | 35 | 25 | 140% |
| 5aS2 A2G1 L-DRG spk-25 | 32.92 | 32 | | | | | |

LOQ: Ct = 36.34

### Example 3: Preparation of Vectors

This example describes methods of preparing and formulating exemplary vectors for gene therapy.

### Genetic Structure of Vector

A vector is made by first replacing both copies of the ICP4 loci by homologous recombination using a plasmid and screening for marker element expressing plaques. A plasmid is constructed by cloning a fragment of a HSV-1 genome comprising regions upstream of the ICP4 promoter including the viral origin contained within the short inverted repeat regions. The plasmid is further modified by cloning a marker element, for example HCMV-eGFP, fragment into the plasmid. This plasmid is then recombined into the ICP4 locus of a wild-type HSV virus. The resulting vector is isolated using a stable ICP4 expressing Vero cell line, such as ‘6-5C’. Vero 6-5C cells are complementing cells that express ICP4.

In order to replace the marker element (e.g., GFP) with a gene of interest (GOI) in the vector described above, a plasmid is constructed by cloning HCMV-GOI-pA into the plasmid. Plaques which do not express the marker element are isolated and tested by ELISA for GOI expression

### Production of Crude Vector

ICP4 complementing Vero cells are cultured in tissue culture flasks using complete media (DMEM supplemented with FBS, HEPES, and Pen Strep) and expanded into 6-12×T175 flasks at a seeding density of 3-4×10⁴ cells/cm². The culture flasks are incubated at 37° C./7.5% CO₂ for 3-4 days.

When cells are 1-2 days over confluent, they are infected at a multiplicity of infection (MOI) of ˜0.1 with a virus stock of known concentration. The infection is initiated by removing the culture supernatant from each flask and infecting with a total of 2.5 mL of complete media containing the appropriate amount of a virus stock. The virus is adsorbed on the cell monolayers by incubating the cultures for 1.5-2 hours, shaking and rotating the flasks every 15-20 minutes. After the adsorption step, an additional 10 mL of complete medium is added to each flask and the cultures are incubated again at 37° C./7.5% CO₂.

Approximately 48 hours after initiating the infection, the flasks are viewed by microscope to confirm cells show signs of cytopathogenic effect and detachment from the flask surface. At that point the cells and supernatant are harvested, pooled together, and centrifuged at ˜1500×g for ˜10 min. The supernatant is removed from the cell pellet and held separately for later processing.

The cell pellet is resuspended in 4-5 mL of complete media, homogenized, and then frozen at −80° C. After the cell suspension has been frozen for >20 minutes, it is thawed and centrifuged at ˜1500×g for ˜10 min. This second cell pellet supernatant is removed and combined with the first collected supernatant.

The pooled supernatant is aliquoted into centrifuge tubes. The virus is then centrifuged at ˜40,000×g for ˜30 minutes at 2-8° C. in order to pellet the virus. After the centrifugation step is completed, the supernatant from the tubes is removed and discarded. The following day the virus pellets are homogenized by pipetting and pooled together. The resuspended virus stock is then aliquoted into cryovials typically at volumes of ˜120 μL per vial. Complete medium (200-300 μL) is added to the virus pellets in order to cover them with liquid and are stored at 2-8° C. overnight to loosen the virus particles. The vials are labeled and frozen at −80° C. Later, a frozen vial is thawed in order to perform a virus plaque titration assay to determine the concentration of the prepared virus stock prior to using in any in vivo or in vitro studies.

### Manufacture of Clarified Vector

Cell Thaw and Expansion

Vero cells (e.g., Vero 6-5, VeroD cells) from a working cell bank are thawed at 37° C. and transferred to a conical tube and pooled. VeroD cells are complementing cells that express or ICP4, ICP27, and UL55. The cells are vialed at approximately 1.0×10⁷ viable cells/mL/tube. The cells are gradually diluted with complete medium and a sample is removed to obtain viable cell counts. The cells are plated in tissue culture flasks at a density of 3.0-5.0×10⁴ cells/cm².

The cells are incubated at 37° C., 7.5%; CO₂ and examined periodically by phase microscopy. The cells are passaged while subconfluent. The complete medium is removed, rinsed with PBS, and the cells are dissociated. The flasks are incubated until the cells detach, then they are re-suspended in complete medium, pooled, counted and seeded into new flasks at a density between 1.0-4.0×10⁴ cells/cm². The cells are expanded and allowed to extend to 1-2 days post-confluence prior to infection.

Infection with Vector

When the cells reach the desired confluence, a model flask is subcultured and the cells are counted to estimate the number of cells per cell factory A master virus bank vector inoculum is prepared by thawing the appropriate volume required to obtain a multiplicity of infection (MOI) of 0.1 and diluting the stock with complete medium up to the target volume desired for the infection. The cell factories are infected by an initial adsorption period followed by incubation for the first day of infection in complete medium. After approximately 24 hours, the culture medium is removed and replaced with an equal volume of serum-free medium. The cell factories are placed in the incubator and the temperature is reduced to 33° C./ with 7.5% CO₂. The cultures are monitored daily and the percent cytopathic effect estimated by visual inspection.

Crude Viral Harvest and Clarification

The infection is stopped by placing the cell factories in a biosafety cabinet and pooling the supernatant and cell debris into a sterile bag. This bulk unclarified harvest is sampled for adventitious agents. After sampling, the sodium chloride level of the harvest is increased and then it is mixed. The harvest is then aliquoted into centrifuge tubes and the cell debris removed by centrifugation. The supernatant is pooled into a sterile bag. After pre-treatment of a clarification filter capsule with sterile water, the virus-containing supernatant is then pumped through the filter capsule into another sterile bag, followed by sterile water to recover remaining virus in the capsule. The bag is mixed and the filtrate was stored overnight at 4° C.

Afterwards, the filtrate is warmed and adjusted to −2 mM MgCl₂ by addition of 2 volumes of 3 mM MgCl₂ in sterile water. The diluted filtrate is mixed and treated with an endonuclease.

Cation Exchange Column Chromatography

A BPG 400 column is packed with SP high performance resin, sanitized with 0.5N NaOH and equilibrated with wash buffer (PBS pH 7.0) and strip buffer (1M NaCl—PBS pH 7.0) before loading endonuclease treated virus.

The process bag containing the endonuclease-treated filtrate is connected to the inlet using a tubing welder and the virus is loaded onto the column. The flow through is collected in a sterile bag. The virus capture step is followed by washing with PBS until the UV absorbance returns to baseline. The pump is stopped and a process bag containing 0.45 M NaCl—PBS (pH 7.0) is connected to the inlet. The outlet tubing is transferred to a sterile container in a biosafety cabinet. The buffer is pumped into the column and when the UV absorbance begins to increase sharply, the column outlet is transferred to a new sterile container to collect the eluted virus. The collection is stopped after the UV absorbance returns to near baseline. This is the purified viral elute fraction. A process bag containing strip buffer is connected to the inlet and the end of the outlet tubing is transferred into a sterile bottle to collect the strip fraction. The buffer is pumped through the column until UV absorbance reaches a peak and returns to near baseline. The collected elute is stored at 4° C. overnight.

Tangential Flow Filtration

The tangential-flow filtration system, using a 0.1 micrometer pore size hollow fiber filter cartridge is prepared by assembling the tubing and cartridge and sterilizing the system by autoclaving. The system is flushed with sterile PBS (pH 7.0) and the virus eluate fraction is added to the system reservoir and equilibrated by recirculation. After equilibration, the permeate collection pump is turned on and filtrate is collected. The system is run until the loaded volume is reduced to approximately 500 ml. The retentate in the reservoir is diluted with DPBS (pH 7.0) with continuous constant volume diafiltration, and the product in the retentate is recovered when the permeate conductivity is within 10% f the diafiltering buffer (DPBS pH 7.0).

Formulation, Final Filtration and Packaging

The recovered retentate is adjusted to 10% final volume with sterile glycerol and mixed well prior to filtering through a 0.45 μm disc filter unit. The product is dispensed into labeled cryovials for storage at ≤−65° C.

### Example 4: Analysis of Transduction of McKrae Strain in DRG after Paw Injection

This example demonstrates that administration of a McKrae strain-based vector results in transduction of dorsal root ganglia (DRG) in vivo.

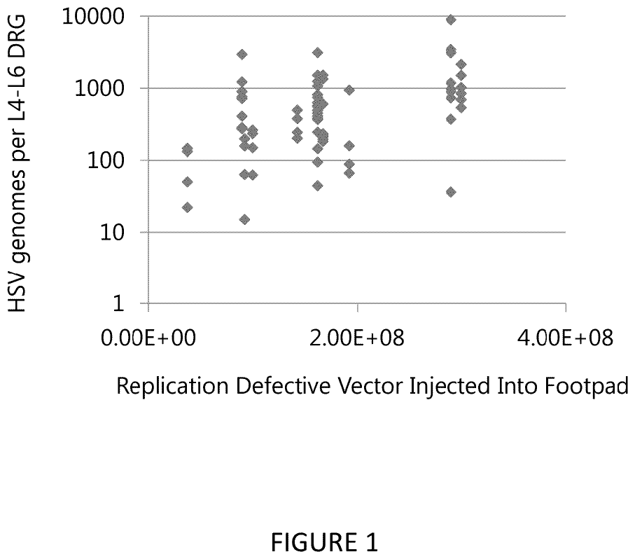

A replication-defective HSV-1 vector as described above was injected into the footpad of rats. As shown in FIG. 1, a replication-defective HSV-1 vector can transduce the DRG neurons in a dose-dependent manner (five days after injection). The ordinate shows a portion of the total number of genomes detectable under assay conditions and indicates that the number of genomes increases relative to dose of vector injected.

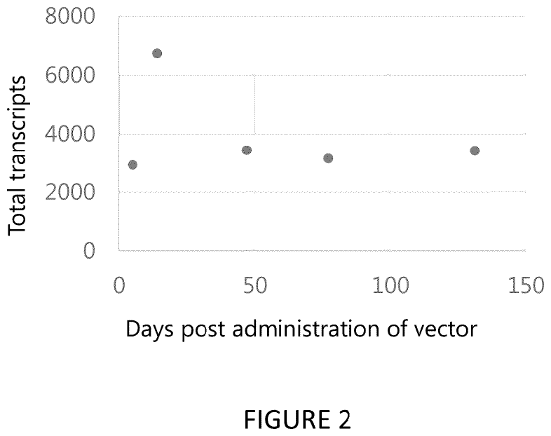

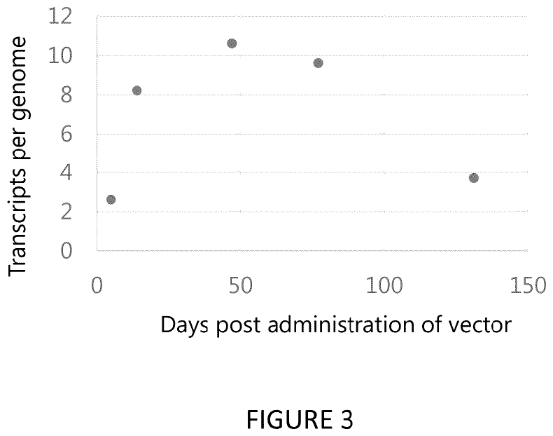

FIG. 2 shows the total number of transcripts of a payload in DRG at 5, 14, 47, 77, and 131 days after injection into the footpad of a rodent. FIG. 3 shows the data from the same experiment as number of transcripts of payload per genome. Expression of the payload was driven by the HCMV promoter.

### Example 5: Analysis of Transduction and Expression of Payload with Different Promoters

This example demonstrates increased gene expression can be obtained in DRG using a neuron specific promoter.

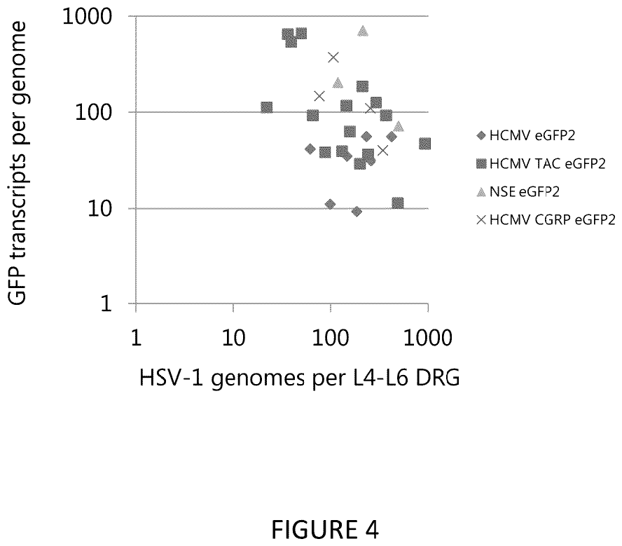

Four different promoters (HCMV, HCMV TAC, NSE and HCMV CGRP) were tested for efficacy in delivering HSV-1 vectors to DRG. The vector comprising a NSE promoter did not have a CMV enhancer, just a neuron-specific promoter. As shown in FIG. 4, a vector with an HCMV promoter averaged 29 transcripts per genome in DRG, while HCMV TAC, NSE and HCMV CGRP promoters averaged 176, 327 and 166 transcripts per genome, respectively.

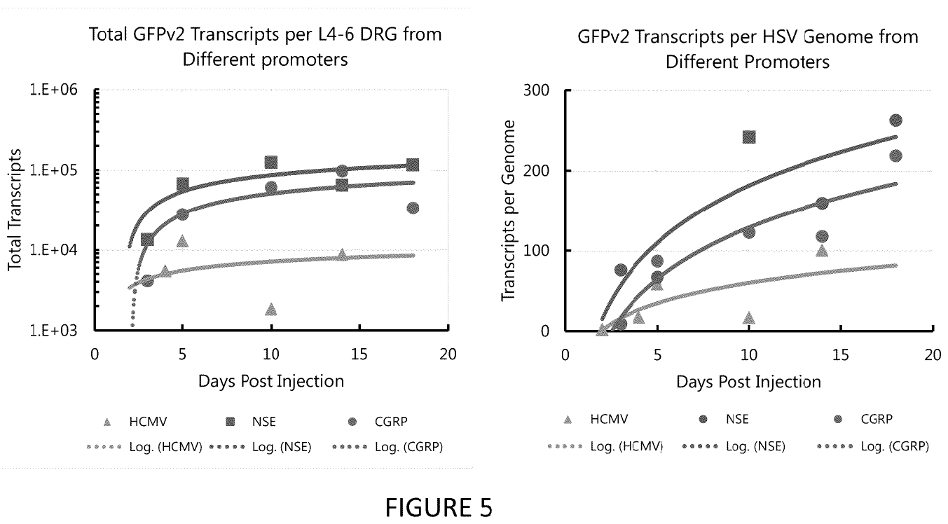

McKrae viral vectors comprising Green Fluorescent Protein (GFP) operatively linked to HCMV, NSE, or CGRP promoters were injected into the footpad of rats and GFP transcripts were measured in L4-L6 DRG over time. As shown in FIG. 5, tissue specific promoters improved transcription in DRG neurons between 5 and 18 days after footpad inoculation.

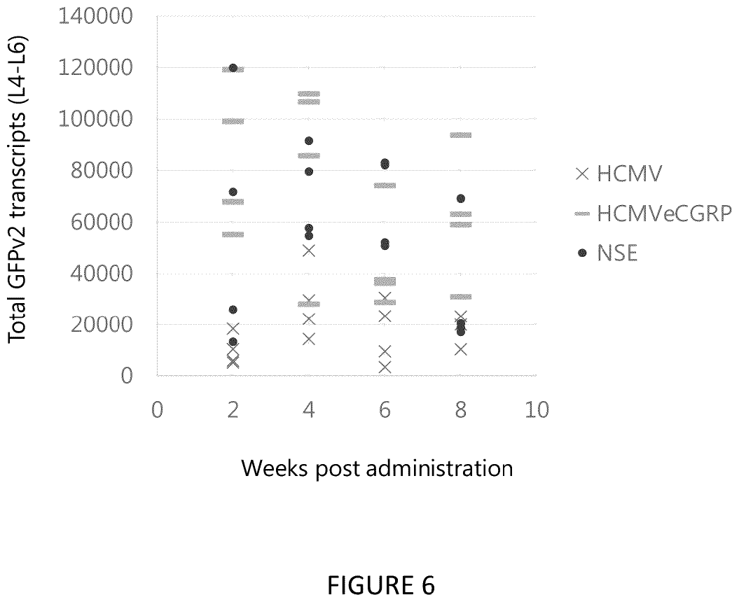

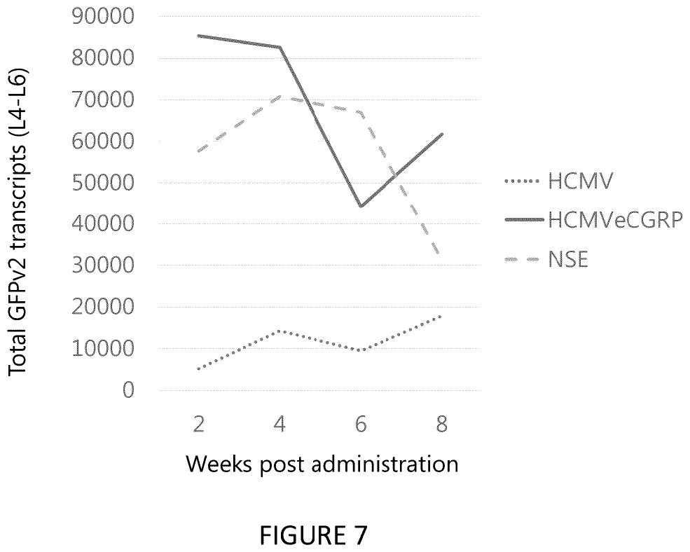

Additionally, when three different promoters (HCMV, HCMVeCGRP and NSE) were compared over time (2-8 weeks), vectors containing either a NSE or HCMVeCGRP promoter resulted in more total transcripts in DRG than a vector containing a HCMV promoter (see FIGS. 6 and 7).

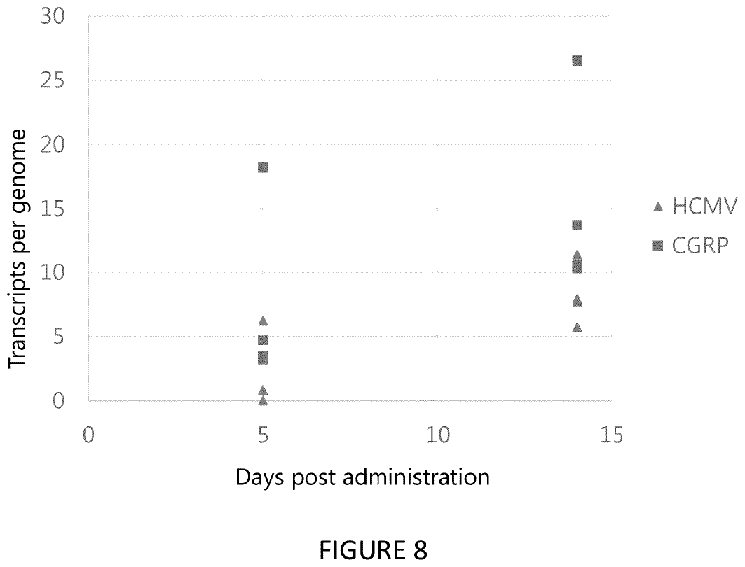

Transcripts of payload were measured in DRG of rats receiving an injection of a McKrae viral vector comprising a polypeptide payload operatively coupled to a CGRP chimeric promoter or an HCMV promoter. As measured at 5 and 14 days post-injection, the CGRP promoter, comprising an HCMV enhancer upstream of the promoter, showed higher transcript numbers of the polypeptide payload per genome than the HCMV promoter (FIG. 8).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1-25. (canceled)

26. A gene therapy vector comprising a replication-defective variant of herpes simplex virus (HSV) McKrae strain whose genome contains an alteration such that the variant fails to express a functional ICP4 protein comprising SEQ ID NO: 16, wherein the gene therapy vector is capable of expressing a payload in a cell susceptible to McKrae strain infection for at least 18 days.

27. The gene therapy vector of claim 26, wherein the gene therapy vector comprises a neuron specific promoter.

28. The gene therapy vector of claim 27, wherein the neuron specific promoter is a calcitonin gene-related peptide (CGRP) promoter.

29. The gene therapy vector of claim 26, wherein the gene therapy vector comprises a human cytomegalovirus (HCMV) enhancer.

30. The gene therapy vector of claim 26, wherein the gene therapy vector comprises a bovine growth hormone (BGH) polyadenylation signal.

31. The gene therapy vector of claim 26, further comprising a nucleic acid that encodes a therapeutic polypeptide.

32. The gene therapy vector of claim 26, wherein the functional ICP4 protein has an amino acid sequence of SEQ ID NO: 2.

33. The gene therapy vector of claim 26, wherein the gene therapy vector is capable of expressing a payload in a cell susceptible to McKrae strain infection for at least 50 days.

34. The gene therapy vector of claim 26, wherein the gene therapy vector is capable of expressing a payload in a cell susceptible to McKrae strain infection for at least 100 days.

35. The gene therapy vector of claim 26, wherein the gene therapy vector is capable of producing in neurons a mean genome copy number of greater than 5,000 transcripts.

36. The gene therapy vector of claim 26, wherein the gene therapy vector is capable of producing in neurons a mean genome copy number of greater than 10,000 transcripts.

37. The gene therapy vector of claim 26, wherein the gene therapy vector is capable of producing in neurons a mean genome copy number of greater than 15,000 transcripts.

38. A cell transduced with a gene therapy vector according to claim 26.

39. A pharmaceutical composition comprising a gene therapy vector according to claim 26 and a pharmaceutically acceptable carrier.


SEQUENCE LISTING (19 sequences)

SEQ ID NO: 1 - DNA, 151135 bp, unidentified
  misc_difference 61968..62069: note = n is a, c, g, or t
  source 1..151135: mol_type = genomic DNA; note = human herpesvirus 1 strain McKrae
gcagcccgggccccccgcgcgcggggcggcgcgcaaaaaaggcgggcggcggtccgggcggcgtgcgcgcgcgcggcgggcgtggggggcggggccgcgggagcgggggaggagccccacccacagacggggaggagcgggggaggagcgggggaggagcgggggaggagccccacccacagacggggaggagcgggggaggagcggccagaccccaaaaacgggcccccccgaaacacaccccccgggggtcgcgcgcggccctttaaagcgcggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggtccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaacgttacacccgaggcgcctgggtcttccgcggagctcccgggagctccgcaccaagccgctctccggagagacgatggcaggagccgcgcatatatacgctgggagccggtccgcccccaaggcgggcccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttggtcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacggtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatgttgggggacccccgacccttaccctggaaccggcccccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacagggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggagagggggaactcgtgggcgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggagggaggaatgggcgggcgggcgaggaaagggcgggccggggagggggcgtaacctgatcgcgccccccgttgtctcttgcagcagactacgtcccgcccgccccccgccggacgccccgcgcccccccacgcagaggcaccgccgcgccccccgtgacgggcggggcgtctaacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtcctcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggccgcaccccctccggcgccgccccgctcccctccgccgtctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataagggacgccccccgtgtttgtggggaggggggtcgggtgctgggtggtctctggccgcgcccactacaccagccaatccgtgtcggggaggggaaagtgaaagacacgggcaccacacaccagcgggtctttagtgttggccctaataaaaaactcaggggatttttgctgtctattgggaaataaaggtttacttttgtatcttttccctgtctgtgttggatggatcttgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgccatgttgggcaggctctggtgttaaccacagagccgcggcccgggctgcctgaccaccgatccccgaaagcatcctgccactggcatggagccagaaccacagtgggctgggtgtgggtgttaagtttccgcgagcgcctgcccgcccggactgacctggcctctggccgccacaaagggcggggggggggttaactacactatagggcaacaaaggacgggaggggtggcgggacggggcgcccaaaagggggtcggccacaccacagacgtgggtgttggggggtggggcggaggggtgggggggagacagaaacaggaacatagttagaaaacaagaatgcggtgcagccagagaatcacaggagacgaggggatgggcgtgttggttaccaacccacacccaggcatgctcggtggtatgaaggagggggggcggtgcttcttagagaccgccgggggacgtggggttggtgtgcaaaggcacgcgcacccgcgtcggccaggtgggccggtaccccatccccccctcccccgacccttccccccccgcgtgccagagatcacccccgtcccccggcacccgccactcctccatatcctcgctttaggaacaactttggggggggggtacacacgcgccgtgcatttccttccacaccccccctcccccgcatccccccccccaggcagtaagacccaagcatagagagccaggcacaaaaacacaggcggggtgggacacatgccttcttggagtacgtgggtcattggcgtggggggttacagcgacaccggccgaccccctggcggtcttccagccggcccttagataagggggcagttggtggtcggacgggtaagtaacagagtctgactaagggtgggagggggggaaaagaacgggctggtgtgctgtaacacgagcccacccgcgagtggcgtggccgaccttagcctctggggcgccccctgtcgtttgggtccccccccctctattggggagaagcaggtgtctaacctacctggaaacgcggcgtctttgttgaacgacaccggggcgccctcgacgagtgggataacgggggaggaagggagggaggagggtactgggggtgaagaaggggggggggaagaagcgagaacaggaaaggcgacggagcccggcagaacaccgaggaaaaaaaaaacacagcgcatgcgccgggccgttgtggggccccgggccggggccccttgggtccgccggggccccgggccgggccgccacgggggccggccgttggcggtaaccccgattgtttatctcaggccccgggccgggaacccggaaaagcctccggggggcctttttcgcgtcgcgtgccggcgagcgggcccggacggggcccggaccgccgcggtcgggggcccctcgtcccgggccgtacgcggccttcgccccgtgaggggccgaacgaacgaaacatcccggcgacggaacgaaaaacaccccagacgggtttaaaaaacagaaaccgtaaccccccccacccccgaaacggggaaaacaaaaaacagaccagcggccggccggcgcttagggggaggatgtcgccgacgccccttggccgccccggctgcaggggggcccggagagccgcggcacccggacgcgcccggaaagtctttcgcaccacccgcgatcggcacggccgcgcccccgcttttataaaggctcagatgacgcagcaaaaacaggccacagcaccacgtgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcgggggggtgtcgcgggccgtctgctggcccgcggccacataaacaatgactcggggcctttctgcctctgccgcttgtgtgtgcgcgcgccggctctgcggtgtcggcggcggctgcggcggctgcggcggccgccgtgttcggtctcggtagccggccggcgggtggactcgcggggggccggagggtggaaggcaggggggtgtaggatgggtatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgggggtgctcgtcgatcgaccgggctcagtgggggcgtggggtgggtgggaaaaggcgaggagactggggtggggggtgtcgggggtggctgtttttttgtggttgttttttgtgtctgttcccgtcccccgtcacccccctccctccgtccccccgtcgcgggtgtttgtgtttgtttattccgacatcggtttatttaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtctcccgcggtgggtgggtgggcggcggtggccgggccgggccgggccgggtgggcggggtttggaaaaacgaggaggaggagaaggaggaggagggggggggagacggggggaaagcaaggacacggccccgggggggggggagcgcgggccgggccgcttggcaacccccctgtttcttccggaaaccaggcttgtggccccacccgacatcacaagggacctcttgtcgggcctcccgacgtacgccgaggctatgtcggaccaccccccaacctaagaggggagaggggagaggggagaggggagaggggagaggggagaggggaggagagggggtatataaaccaacgaaaagcgcgggaacggggatacggggcttgtgtggcacgacgtcgtggttgtgttactgggcaaacacttggggactgtaggtttctgtggtgccgaccctaggcgctatggggattttgggttgggttgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgctacgagaccccctcggctataaactatgctttgatagacggtatatttttgcgttatcactgtcccggattggacacggtcttgtgggataggcacgcccagagggcgtattgggttaacccctttttgtttggggcgggttttttggaggacttgagtcatcccgcgtttcctgccgacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgccaggatttgggacttaccaacagaacgtctggacggaccccggttctgccgtcggacgatgaagcgggcctgcagccgaagcccctcaccacgccgtcgcccatcatcgccacgtcggaccccaccccgcgacgggacgccgccacaaaaagcagacgccgacgaccccattcccggcgcatctaatgatgcctcgacggaaaaccgtccgggtttggggggcgaaccggccgcctgtcgctcgtcagggccggcgggcgctcctcgccgccctagaggctgtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgttggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgcttcgtgggcggagttatccgccggttggctgcgcgccgccccggcctggtgtttatgctctggggcgcacatgcccagaatgccatcaggccggaccctcgggtccattgcgtcctcaagttttcgcacccgtcgcccctctccaaggttccgttcggaacatgccagcatttcctcgtggcgaatcgatatctcgagacccggtcgatttcacccatcgactggtcggtttgaaaggcatcgacgtccggggttttcgtctgtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcagggatatcgccagatacgactcccgcagattccattcggggggccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagccttcagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccgcgccaccctcactgcccgtcgcgcgtgtttgatgttaataaataacacataaatttggctggttgtttgttgtctttaatggaccgcccgcagggggggtggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggtcgtcgggagtggaggcgcggcgcccaccccgtgtgccgcgcctggcgtctcctctgggggcgacccgtaaatggttgcagtgatgtaaatggtgtccgcggtccagaccacggtcaaaatgccggccgtggtgctccgggcgctttcgccgcgcgaggagctgacccaggagtcgaacggatacgcgtacatatgggcgtcccacccgcgttcgagcttctggtcgctgtcccggcctataaagcggtaggcacaaaattcggcgcgacagtcgataatcaccaacagcccaatgggggtgtgctggataacaacgcctccgcgcggcaggcggtcctggcgctcccggccccgtaccataatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtagaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgcgtacgtgtatcccatgagcgtataattgtccgtctgcagggcgacggacatcagccccccgcgcggcgagccggtcagcatctcgcagccccggaagataacgttgtccacgtacgtgctaaagggggcgccttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggctattaccgacgcctgcgtcggcgcccccgaagatttggggcgcagagacagaatctccgccgttagttctcccatgcgggcgtaggcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggcgttggccgtgagccacttttcaatagtcaggccgggctggtgtgtcagccgtcggtattcgtcaaactccttgaccgacacgaacgtaagcacggggagggtgaacacgacgaactccccctcacgggtcaccttcaggtaggcgtggagcttggccatgtacgcgctcacctctttgtgggaggagaacagccgcgtccagccggggaggttggcggggttggtgatgtagctttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcggttgcagatgaggtacgtgagcacgttctcgctctggcggacggaacaccgcagtttctggtgctcgaaggtcgactccagggacgccgtctgcgtcggcgagcccacacacaccaacacgggccgcaggcgggccgcatactggggggtgtggtacagggcgttaatcatccaccagcaatacaccacggccgtgaggaggtgacgcccaaggagcccggcctcgtctatgacgatcacgttgctgcgggtaaaggccggcagcgccccgtgggtggccggggccaaccgcgtcagggcgccctcggccaaccccagggtccgttccagggcggccagggcgcgaaactcgttccgcgactcctcgcccccggaggcggccagggcgcgcttcgtgaggtccaaaatcacctcccagtagtacgtcagatctcgtcgctgcaggtcctccagcgaggcggggttgctggtcagggtgtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagacggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtctctgtccgtctagctggcgctccccgccggccgccgccatgaccgcaccacgctcgcgggcccccactacgcgtgcgcggggggacacggaagcgctgtgctcccccgaggacggctgggtaaaggttcaccccacccccggtacgatgctgttccgtgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggcctcccttgtggaccggggaccgtctggtcttcgattcgcccgtccagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggcgccgcgcgggtcgttaacaaggtgttgggggaggacgacgagaccaaggccgggtcggccgcctcacgcctcgtgcggcttatcatcaacatgaagggcatgcgccacgtaggcgacattaacgacaccgtgcgtgcctacctcgacgaggccggggggcacctgatagacgccccggccgtcgacggtaccctccctggattcggcaagggcggaaacagccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacatgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgaggcagtgcttcgggccccccgggtaggcgcgcccatcacgggctccgatgtcatcctgggggaggaggagttatgggatgcggtgtttaagaaaacctgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgcgaggcgcaccggaccagggcgggggcatcgggcaccgggatggccgccgcgacggccgacgatgaggggtcggccgtcaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcatccgcagttgccactgggtcctaaacacactcatgttcatggtgcacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttagtaaataaacccggttttgtttctatggcctcctgacggatgcgcgtgtccttactccgttttggtgggtgggtggctgtgtatggcgtcccatctgtgcggggagggggcaagtcggcacgtattcggacagactcaagcacacacgggggagcgctcttggctcagggcaatgtttttattggtcaaactcaggcaaacagaaacaacatcttgtcgtcaaagggatacacaaacttccccccctcgccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccaacaaggcgccgccgcggaaggcagtggggggcaaggggtcgctggcctcaaagggggacacccgaacgctccagtactccgcgtccaaccgtttattaaacgcgtccacgataaggcggtcgcaggcgtcctccataaggccccgggccgtgagcgcgtcctcctccggcacgcctgccgttgtcaggcccaggacccgtcgcagcgtgtcgcgtacgaccccggccgccgtggtgtacgcgggcccgcggagaggaaatcccccaagatggtcagtgttgtcgcgggagttccagaaccacactcccgcttggctccaggcgacggcgtgggtgtagacgccctcgagcgccaggcacagtgggtgccgcagccggaggccgttggccataagcacggctcccacggccgtctcgatggcccgccgggcgtcctcgatcaccccggaagccgcatccgcgtcttgggggtccacgttaaagacaccccagaacgcacccccatcgcccccgcagaccgcgaacttaaccgagctggccgtctcctcaatctgcaggcagacggcggccatcaccccgcccaggagctgccgcagcgcagggcaggcgtcgcacgtgtccgggaccaggcgctccaagacggccccggcccagggctctgagggagcggccaccaccagcgcgtccagtcttgctaggcccgtccggccgtggggttccgccagcccgctccccccgaggtcggccagggccgccaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgctccctccttcttgatcggcggaaacgcaagagtctggtattcgcgcgcaaatagcgcggttccggtggtgatgttaacggtcagcgaagcggcggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacgggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcgggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtggttcaggagcatgggcgtaaagacgggctccgagcgcgccccggagccgtccatgtaaatcagtagctcccccttgcggagggtgcgcacccgtcccagggactggtacacggacaccatgtccggtccgtagttcatgggtttcacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgatgaaaaaatgcagatgttatcgcccccgccaaggcgcgcttccagctccccaaagaacgtggccccccgggcgtccggagaggcgtccggagacgggccgcttggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacaggtgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgccagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggcccccagaggatcccccgactccgcgccccccacgaaaggcatgaccggggcgcggacggcgtggtgggtctggtgtgtgcaggtggcgacgtttgtggtctctgcggtctgcgtcacggggcttctcgtcctggcctctgtgttccgggcacggtttccctgcttttatgccacggcgagctcttatgccggggtgaactccacggccgaggtgcgcgggggtgtagccgtgcccctcaggttggacacgcagagccttgtgggcacttatgtaatcacggccgtgttgttgttggccgcggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcatcagccagctggcccacctggtttacgtcctgcactttgcgtgtctggtgtattttgcggcccatttttgcaccaggggggtcctgagcgggacgtatctgcgtcaggtgcacggcctgatggagccggccccgactcatcatcgcgtcgtcggcccggctcgagccgtgctgacaaacgccttgctgttgggcgtcttcctgtgcacggccgacgccgcggtatccctgaataccatcgccgcgttcaactttaatttttcggccccgggcatgctcatatgcctgaccgtgctgttcgcccttctcgtcgtatcgctgttgttggtggtcgagggggtgttgtgtcactacgtgcgcgtgttggtgggcccccacctgggggccgtggccgccacgggcatcgtcggcctggcctgcgagcactattacaccaacggctactacgttgtggagacgcagtggccgggggcccagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaggcgcgtacgcagttccatgcgcggatcgcgagacggccgccacaggcccgcacccggcagcccgcccgggattcccgaatatgcggaagacccctacgcgatctcatacggcggccagctcgaccggtacggagattccgacggggagccgatttacgacgaggtggctgacgaccaaaccgacgtattgtacgccaagatacaacacccgcggcacctgcccgacgacgagcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgtttcccccccccccccctcaaccccggcgattgtgggttttttaaaaacgacacgcgtgcgaccgtatacagaacattgttttggtttttattcgctatcggacatggggggtggaaactgggtggcggggcaggcgcctccgggggtccgccggtgagtgtggcgcgagggggggtccgacgaacgcaggcgctgtctccccggggcccgcgtaaccacgcgcatatccgggggcacgtagaaattaccttcctcttcggactcgatatccacgacatcaaagtcgtgggcggtcagcgagacgacctccccgtcgtcggtgatgaggacgttgtttcggcagcagcagggccgggccccggagaacgagaggcccatagctcggcgagcgtgtcgtcgaacgccaggcggctgcttcgctggatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctggtgcggtgcctgccgataaacgtcaccagatgcgggtggggggggcagtcggggaagtggctgtcgagcacgtagccctgcaccaagatctgcttaaagttcgggtgacgggggttcgcgaagacgggctcgcggcggaccagatccccggagctccaggacacgggggagatggtgtggcgtccgaggtcgggggcgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgtcatcaagtcctcgtacgcggaggccgtggggtcgctggggtccatggggtcgaaagcgtacttggcccggcatttgacctcgtaaaaggccaggggggtcttggggactggggccaggtagccgtgaatgtcccgaggacagacgagaatatccagggacgccccgaccatccccgtgtgaccgtccatgaggaccccacacgtatgcacgttctcttcggtgaggtcgctgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggcccgcggcgcgtcaggcgccataggtgccggccgagtccgcggtccaccatacccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggccctgaggcgtcagaaagtgccggcgacgcggcccggggcttttcgtctgcggtgtcggtggcgtgctgatcacgtggggggttaacgggcgaatgggagctcgggtccacagctgacgtcgtctggggtggggggggcaggggacggaaggtggttgtcagcggaagactgttagggcgggggcgcttgggggggctgtcggggccacgaggggtgtcctcggccagggcccagggacgcttagtcacggtgcgtcccggcggacatgctgggcctaccgtggactccatttccgagacgacgtgggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctggtacgtcatacgccaggccgtgggtgttggtctcggccgagttgacaaacagggctgggtgcagcacgcagcgataggcgagcagggccagggcgaagtccggcgacagctggttgttgaaatactggtaaccgggaaaccgggtcacgggtacgcccaggctcggggcgacgtacacgctaaccaccaactccagcagcgtctggccaagggcgtacaggtcaaccgctaacccgacgtcgtgcttcaggcggtggttggtaaattcggcccgttcgttgttaaggtatttcaccaacagctccgggggctggttatacccgtgacccaccagggtgtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcacttgatatccaggtggctgatcccgcaggtggtgtttaaaaacacaacggcgcgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagagcgcactccccgaccaacagggtcgcgatgagctcaacggcaaaccactccttttcctttatggtcttaacggcaagcttatgttcgcgaatcagttggacgtcgccgtatcccccagaccccccgaagcttcgggccccggggatctcgagggtcgtgtagtgtagggcggggttgatggcgaacacggggctgcatagcttgcggatgcgcgtgagggtaaggatgtgcgagggggacgaggggggtgcggttaacgccgcctgggatctgcgcaggggcgggcggttcagtttggccgccgtaccgggcgtctcgggggacgcgcggcgatgagacgagcggctcattcgccatcgggatagtcccgcgcgaagccgctcgcggaggccggatcggtggcgggacccgtgggaggagcgggagccggcggcgtcctggagagaggggccgctggggcgcccggaggccccgtgtgggttggagtgtatgtaggatgcgagccaatccttgaaggaccgttggcgtgcaccttgggggctgaggttagctgccacatgaccagcaggtcgctgtctgcgggactcatccatccttcggccaggtcgccgtctccccacagagaagcgttggtcgctgcttcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgctgacgccctcccggtgcagctgcagggtctggtccttgtaaatctcggctcggaggtgcgtctcggccaggcgtcggcgcagggccgcgtgggcggcatctcggtccattccgccaccctgcgggcgacccggggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcacaatgggcggcgatgccctacgagtgccctttttagatttcgcgaccgcgacccccaagcgccaccagaccgtggtcccgggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaacacgtttcaggccctccaccgctccgaagcctttcgccagctggtgcactttgtgcgggactttgcccagctgcttaaaacctccttccgggcctccagcctcacggagaccacgggcccccccaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctagtgcccctcatcgcgctgtcactggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacccacatacacacacaaccagacgcgggaggaaagtctgccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttggtgatcctggccaggagccatcccagatggcgggccctgcttcccggtggacagagcgaccccaggtcgctgtccatggcccagcagtagatctggccgctggggaggtgccaccaggcccccgggcccaaggcgcagcacgcgcccggctccgggggggtcttcgcggggaccagatacgcgccatccagctcgccgaccactggctcctccgcgagctgttcggtggttgggtcgggggtttcctccgggggggtggccgcccgtatgcgggcgaacgtgagggtgcacaggagcggggtcagggggtgcgtcacgctccggaggtggacgatcgcgcagtagcggcgctcgcggttaaagaaaaagagggcaaagaaggtgttcgggggcaaccgcagcgccttggggcgcgtcagatacagaaaaatctcgcagaagagggcgcgcccggggtctgggttaggaagggccacctgacacagaggctcggtgaggaccgttagacaccgaaagatcttgagccgctcgtccgcccgaacgacgcgccacacaaagacggagttgacaatgcgcgcgatagagtcgacgtccgtccccaggtcgtcgactctgtcgcgcgtgccgcgagctccggcccgggaatccggccggggcaaggtccccgggggaccaggcggcgccaggggccgccggggtcccagctgcgccatgccgggggcggggggagggcaaaccccagaggcgggggccaacggcgcggggaggagtgggtgggcgaggtggccgggggaaggcgcccgctagcgagaacggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcaccatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggagtcgggggcggtcccctcgacggcgcacatcaggttctcgaggagaatggggaagcgcctggtcacctcccccaaccacgcgcgttggtcggggccaaagtcatagcgcaggcgctgtgagattcgcgggccgccctgaagcgcggcccggatggcctggcccagggcccggaggcacgccagatgtatgcgcgcggtaaaggcgacctcggcggcgatgtcaaagggcggcaggacggggcgcgggtggcgcaggggcacctcgagcgcgggaaagcgtagcagcagctccgcctgcccagcgggagacagctggtgggggcgcacgacgcgttctgcggcgcaggcctcggtcagggccgtggccagcgccgaggacagcagcggagggcgggcgcgtcgcccgccccacgccacggagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgcggtaccaggggtcagggttagaacgtcgttcaggatgtcctcgcccccgggcccgtggggcacgggggccacaaagcggcccccgcctgggggctccagacccgccaacaccgcatctgcgtcagccgcccccatggcgcccccgctgacggcctggtgaaccagggcgccctggcggagccccgatgcaacgccacaggccgcacgcccggtccgagcgcggaccgggtggcggcgggtgacgtcctgcactgcccgctgaaccaacgcgaggatctcctcgttctcctgcgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcagcctccttcgcggccggcggggcggcggcgccaagcagagcgacgtagcggcacaaatgccgacagacgcgcatgatgcgcgtgctgtcggccgcgtagcgcgtgttgggggggacgagctcgtcgtaactaaacagaatcacgcgggcacagctcgcccccgagccccacgcaaggcgcagcgccgccacggcgtacgggtcatagacgccctgcgcgttacacaccacgggcagggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggtggccgcgcatcagggcggccagagggattcccgcggccgccaggcactcgctggatatgatgacgtgaaccaaagacgagggccgacccgggccgtggccgagatcgtactggacctcgttggccaagtgcgcgttcatggttcgggggtgggtgtgggtgtgtaggcgatgcgggtcccccgagtccgcgggaagggcgtgggtttggcgcgcgtatgcgtattcgccaacggaggcgtgcgtgcttatgcgcggcgcgtttcttctgtctctagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgcagacgagcttctcaacgtggtgacctatatatgcgatgatcacatgccgagggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgacgcaggtcctggccctgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctaaataacctcaccgaaaccgtctcccccaacactgacgtccgtacgtattccggaaaacggaacggcgcctcggatgaccttatggtcgccgtcattatggccatctacctcgcggcccaggccggacctccgcacacattcgctcctatcacacgcgtcttgtgagcgcccaataaacacacccaggtatgctacgcacgaccacggtgtcgtctgttaaggggggggggaagggggtgttggcgggaagcgtgggaacacgggggattctctcacgaccggcaccagtaccacccccctgtgaacacagaaaccccaacccaaatcccataaacatacgacacacaggcatattttggaatttcttaggtttttatttatttaggtatgctggggtttctccctggatgcccacccccacccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtaaagcggcagccttcgctctccatcctgcatcagggccggggcttcggggtgaatgagctgggcggcctcccgcgtgacactctgcatctgcaggagagcgttcacgtacccgtcctgggcacttagcgcaaagagccgggggattagcgtaaggatgatggtggttccctccgtgatcgagtaaaccatgttaaggaccagcgatcgcagctcggcgtttacgggaccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccagggggcaggcgacgattcgcgtgggaagcacccgggtgatgaccgcggggaagcgccttcggtacgccagcaacaaccccaacgtgtcgggactgacgcctccggagacgaaggattcgtgcgccacgtcggccagcgtcagttgccggcggatggtcggcaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtgggtcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgtccgcggagtcccggcttgggttttatggggcccggccctcggaatcgcggcttgtcggcggggacaaagggggcggggctaggggcttgcggaaacagaagacgcgtgggataaaagaatcgcactaccccaaggaagggcggggcggtttattacagagccagtcccttgagcggggatgcgtcatagacgagatactgcgcgaagtgggtctcccgcgcgtgggcttccccgttgcgggcgctgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcgaaggcccgggacgggtcgctctggccgtggtcgtacatgagggcggtgacatccccctccttgtcccccgcgtaaacgcccccggcggcgcgtccccgggggttgcagggccggcggaagtagttgacgtcggtcgacacgggggtggcgataaactcacacacggcgtcctggccgtggtccatccctgcgcgccgcggcacctgggcgcacccgaacacggggacgggctgggccggccccaggcggtttcccgccacgaccgcgttccgcaggtacacggctgccgcgttgtccaggagagggggagccccgcggcccaggtaaaagttttggggaaggttgcccatgtcggtgacggggttgcggacggttgccgtggccacgacggcggtgtagcccacgcccaggtccacgttcccgcgcggctgggtgagcgtgaagtttacccccccgccagtttcgtgccgggccacctggagctggcccaggaagtacgcctccgacgcgcgctccgagaacagcacgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgtggacggggagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccgggtgcagggggtgcgcggggtccgtcacggggtcgctggggcactcctcgccgggggcgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcggccggtcatccgcggcgtccgcggctcgggcctgggtgttgtgcagcagctggccgtcgttgcggttgaagtccgcggtcgccacgttacatgccgccgcgtacacggggtcgtggccccccgcgctaacccggcagtcgcgatggcggtccagggccgcgcgccgcatcagggcgtcacagtcccacacgaggggtggcagcagcgccgggtctcgcattaggtgattcagctcggcttgcgcctgcccgcccagctccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggaccacgagccgggccagggcgcagaagacgtgctcgctgccgtgtatggcggcctgcagcaggtaaaacaccgccgggtagttgcggtcgtcgaacgccccgcgaacggcggcgatggtggcgggggccatggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacaaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggttaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggactaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccagaaagaccagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccgctttcaggaaggactgcaggaggcgctgtttgatcggggcggtggtgacgagcaccccgtcgaccggccgcccgcgcgtgtcggcatgcgtcagacggggcacggccacggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagctgttggatggcgcgcaactggcgatgcgcgccgatacccgtcccgtccagggcctcccccgtgagcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaacccgagctccagaaagcgcacgagcgacagggtgttgcaatacgaccccagcagggcgtcgaactcgacgtcgtacaggctgtttgcatcggagcgcacgcgggaaaaaaaatcgaacaggcgtcgatgcgacgccacctcgatcgtgctaaggagggacccggtcggcaccatggccgcggcataccggtatcccggagggtcgcggttgggagctgccatggggtcgcgtggagatcggctggatctagcgatatttgcccggggaggctaagatccaccccaacgcccggccacccgtgtacgtgcccgacggcccaaggtccaccgaaagacacgacgggcccggacccaaaaaggcgggggatgctgtgtgaggggccgggtgtcggtcgggggggaaaggcaccgggagaaggctgcggcctcgttccaggagaacccagtgtccccaacagacccggggacgtgggatcccaggccttatataccccccccgccccacccccgttagaacgcgacgggtgcattcaagatggccctggtccaaaagcgtgccaggaagaaattggcagaggcggcaaagctgtccgccgccgccacccacatcgaggccccggccgcgcaggctatccccagggcccgtgtgcgcaggggatcggtgggcggcagcatttggttggtggcgataaagtggaaaagcccgtccggactgaaggtctcgtgggcggcggcgaacaaggcacacagggccgtgcctcccaaaaacacggacatcccccaaaacactggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaaatcagaggtgccgtacgaggacaatgaaaactgttcctccagcggcagttctccctcctcccccccgaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaacccccccgtgggtgtgacgttgcgtttagttcattggaggccaaggggaaaaatggggtggggaggaaacggaaaacccagtaggccgtgtcgggaacacgcccggggttgtcctcaaaaggcagggtccatactacggaagccgtcgttgtattcgagacctgcctgtgcgacgcacgtcggggttgcctgtgtccggttcggcccccaccgcgtgcggcacgcacgaggacgagtccgcgtgctttattggcgttccaagcgttgccctccagtttctgttgtcggtgttcccccatacccacgcccacatccaccgtagggggcctctgggccgtgttacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctggccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctggttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaaaagaccccaataaacgtatgtcgctactacacccttgtgtgtcaatggacgcctctccgggggggggaagggaaagcaaagaggggctgggggagcggcaccaccggggcctgaacaaacaaaccacagacacggttacagtttattcggtcgggcggagaaacggccgaagccacgcccactttattcgcgtctccaaaaaaacgggacacttgtccggagaacctttaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgcttggggcgccctccgtcggcccggcggcgatttgctgctgtgtgttgtccgtatccaccagcaacacagacatgacctccccggccggggtgtagcgcataaacacggcccccacgagccccaggtcgcgctggttttgggtgcgcaccagccgcttggactcgatatcccgggtggagccttcgcatgtcgcggtgaggtaggttaggaacagtgggcgtcggacgtcgacgccggtgagcttgtagccgatcccccggggcagaggggagtgggtgacgacgtagctggcgctgtgggtgatgggtaccaggatccgtggctcgacgttggcagactgccccccgcaccgatgtgaggcctcagggacgaaggcgcggatcagggcgttgtagtgtgcccaacgcgtcagggtcgaggcgaggccgtgggtctgctgggccaggacttcgaccggggtctcggatcgggtggcttgagccagcgcgtccaggataaacacgctctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacagggccgaggctatcagaaggctccggcgggcgcgttcccgctgcaccgccgaggggacgccagccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagaggggggcgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgctcgggtggctcgccgtcgccacgttgtctccccgcgggaacgtcagggcctcggggtcagggacggccgaaaacgttacccaggcccgggaacgcagcaacacggaggcggctggattgtgcaagagacccttaaggggggcgaccgaggggggaggctgggcggtcggctcgaccgtggtgggggcgggcaggctcgcgttcgggggccggccgagcaggtaggtcttcgggatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggcccgtggctatggcagggcttgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttacttccggtattgtctccttccgtgtttcagttagcctcccccatctcccgggcaaacgtgcgcgccaggtcgcagatcgtcggtatggagccgggggtggtgacgtgggtctggaccatcccggaggtaagttgcagcagggcgtcccggcagccggcgggcgattggtcgtaatccaggataaagacgtgcatgggacggaggcgtttggccaagacgtccaaggcccaggcaaacacgttatacaggtcgccgttgggggccagcaactcgggggcccgaaacagggtaaataacgtgtccccgatatggggttgtgggcccgcgttgctctggggctcggcaccctggggcggcacggccgtccccgaaagctgtccccaatcctcccgccacgacccgccgccctgcagataccgcaccgtattggcaagcagctcgtaaacgcggcgaatcgcggccaacatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtaacacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccaacacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccaggcggacttccgtggctttttgctgccggcgagggcgcaacgccgtacgtcggttgttatggccgcgagaacgcgcagcctggtcgaacgcagacgcgtgttgatggcaggggtacgaagccatacgcgcttctacaaggcgctggccgaagaggtgcgggagtttcacgccaccaagatctgcggcacgctgttgacgctgttaagcgggtcgctgcagggtcgctcggtattcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccattgagcctaggacccagcgagcccgtcgccgccgcggcggcgccgcccgggggtctgcgagcagaccgaaaaggtcacactctggggcgcgcgacccgcccgagtcagcggcccgccagttaccacccgccgaccaaacccccgcctccacggagggcgggggggtgcttaagaggatcgcggcgctcttctgcgtgcccgtggccaccaagaccaaaccccgagccgcctccgaatgagagtgtttcgttccttccccctccccccgcgtcagacaaaccctaaccaccgcttaagcggcccccgcgaggtccgaagactcatttggatccggcgggagccacccgacaacagcccccgggttttcccacgccagacgccggtccgctgtgccatcgcgccccctcatcccaccccccatcttgtccccaaataaaacaaggtctggtagttaggacaacgaccgcagttctcgtgtgttattttcgctctccgcctctcgcagatggacccgtactgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacacatggtgtacgcgggccgcggggcgaccggctcgtcgggggtggtgttcgggacctggtaccgcactatccaggaccgcaccatcacggactttcccctgaccacccgcagtgccgactttcgggacggccggatgtccaagaccttcatgacggcgctggtcctgtccctgcagtcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagtacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccccgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaagaacaagagcctccgttgggttttctttgtgcctgtactcaaaaggtcataccccgtaaacggcgggctccagtcccggcccggtggttggcgtgaacgcaacggcgggagctgggttagcgtttagtttagcattcgctctcgcctttccgcccgccccccgaccgttgcgccttttttttcgtccaccaaagtctctgtgggtgcgcgcatggcagccgatgccccgggagaccggatggaggagcccctgcccgacagggccgtgcccatttacgtggctgggtttttggccctgtatgacagcggggactcgggcgagttggcattggatccggatacggtgcgggcggccctgcctccggataacccactcccgattaacgtggaccaccgcgctggctgcgaggtggggcgggtgctggccgtggtcgacgacccccgcgggccgttttttgtggggctgatcgcctgcgtgcagctggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtcgcgctgtgcgcgatcgggcggcgcctcggcactatcgtcacctacgacaccggtctcgacgccgccatcgcgccctttcgccacctgtcgccggcgtctcgcgagggggcgcggcgactggccgccgaggccgagatcgcgctgtccgggcgcacctgggcgcccggcgtggaggcgctgacccacacgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacataccgcccggctcgatcgctgccgcgccgcagggtgaccggtgcccaatcgtccgtcagcgcggggtcgccttgtccccggtactgccccccatgaaccccgttccgacatcgggcaccccggcccccgcgccgcccggcgacgggagctacctgtggatcccggcctcccattacaaccagctcgtcgccggccatgccgcgccccaaccccagccgcattccgcgtttggtttcccggctgcggcgggggccgtggcctatgggcctcacggcgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgggccgcgcggggtcgactctcggcgcgcggcccgccagtctcccgggaccaacgagaccatcacggcgctgatgggggcggtgacgtctttgcagcaggaactggcgcacatgcgggctaggaccagcgccccctatgggatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaagaagacccattggtgtatgttctttttttattgggaggcgcgggtaggcgggtagctttacaatgcaaaagccttcgacgtggaggaaggcgtgggggggaatcggcactgaccaagggggtccgttttgtcacgggaaaggaaagaggaaacaggccgcggacacccgggggagtttatgtgttcccttttctttcttcccacacacacaaaaggcgtaccaaacaaacaaaccaaaagatgcacatgcggtttaacacccgtggtttttatttacaacaaaccccccgtcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggcagacaccagggccatgtaccgtatcatctcccgggcctcggctagcttggcctcgtcaaagtcgccgccctcctcgccctccccggacgcgtccgggttggtggggttcttgagctccttggtggttagcgggtacagggccttcatggggttgctctgcagccgcatgacgtagcgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcaaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtacacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcacgcacgtggagacggccatcacgtcgccgagcatccgcgcgctcacccgccggcccacggtggccgaggcgatggcgttggggttcagcttgcgggcctcgttccacagggtcagctcgtgattctgcagctcgcaccacgcgatggcaacgcggcccaacatatcgttgacatggcgctgtatgtggttgtacgtaaactgcagcctggcgaactcgatggaggaggtggtcttgatgcgctccacggacgcgttggcgctggccccgggcggcgggggcgtggggtttgggggcttgcggctctgctctcggaggtgttcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaggagaatcggaaggagccgccgtactcggagcgcagcatctcgtccacctcctgccacttggtcatggtgcagaccgacgggcgctttggcacccagtcccaggccacggtgaacttgggggtcgtgagcaggttccgggtggtcggcgccgtggcccgggccttggtggtgaggtcgcgcgcgtagaagccgtcgacctgcttgaagcggtcggcggcgtagctggtgtgttcggtgtgcgacccctcccggtagccgtaaaacggggacatgtacacaaagtcgccagtcgccagcacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgcggcggctttgggggtttcggttttttgttcttcttcggtttcgggtcccccgttgggggggcgccaggggcgggcggcgccggagtggcagggcccccgttcgccgcctgggtcgcggccgcgaccccaggcgtgccgggggaactcggagccgccgacaccaccaggacccccagcgtcaaccccaagagcgcccatacgacgaaccaccggcgcccccgcgcgggggcgccctggcgcatggcgggactacgggggcccgtcgtgccccccgtcaggtagcctgggggcgaggtgctggaggaccgagtagaggatcgagaaaacgtctcggtcgtagaccacgaccgaccgggggccgatacagccgtcgggggcgctctcgacgatggccaccagcggacagtcggagtcgtacgtgagatatacgccgggcgggtaacggtaacgaccttcggaggtcgggcggctgcagtccgggcggcgcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagcggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgcacgtccgcggccgcttgcgtggcgatgtccccccacgtctcgggcccctgccccccgggcccgcggcgacggtcttcgtcctcgcccccgcccccgggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcattctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggtggcagtagtttagctgctttagaaagttctcgacgtcgtgcaggaaacacagctccatatggacggtcccgccatacgtatccagcctgacccgttggtgatacggacagggtcgggccaggcccatggtctcggtgaaaaacgccgcgacgtctcccgcggtcgcgaacgtctccaggctgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgcccgagcagggctaacaacttttgacgggccacggtgggctcggacaccggggcggccatctcgcggcgccgatctgtaccgcggccggagtatgcggtggaccgaggcggtccgtacgctacccggcgtctggctgagccccggggtccccctcttcggggcggcctcccgcgggcccgccgaccggcaagccgggagtcggcggcgcgtgcgtttctgctctattcccagacaccgcggagaggaatcacggcccgcccagagatatagacacggaacacaaacaagcacggatgtcgtagcaataatttattttacacacattccccgccccgccctaggttcccccacccccaacccctcacagcatatccaacgtcaggtctccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacacctccccggcgttgccgagttggctgactagggcctcggcctcgtgcgccacctccagggccgcgtccgtcgaccactcgccgttgccgcgctccagggcacgcgcggtcagctccatcatctcctcgcttaggtactcgtcctccaggagcgccagccagtcctcgatctgcagctgctgggtgcggggccccaggcttttcacggtcaccacgaacacgctactggcgacggccgccccgccctcggagataatgccccggagctgctcgcacagcgagctttcgtgcgctccgccgccgaggctcgaggccgcgcacacaaacccggcccggggacaggccaggacgaacttgcgggtgcggtcaaaaataaggagcgggcacgcgtttttgccgcccatcaggctggcccagttcccggcctgaaacacacggtcgttgccggccatgccgtagtacttgctgatgctcaaccccaacacgaccatggggcgcgccgccatgacgggccgcagcaggttgcagctggcgaacatggacgtccacgcgcccggatgcgcgtccacggcgtccatcagcgcgcgggccccggcctccaggcccgccccgccctgcgcggaccacgcggccgcagcctgcacgctggggggacggcgggaccccgcgatgatggccgtaagggtgttgatgaagtacgtcgagtgatcgcagtaccgcagaatctggtttgccatgtagtacatcgccagctcgctcacgttgttgggggccaggttaataaagtttatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgttaggctgggggcgcagatagccgccccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactggctcagggccaggtcctgatacacggcgaggttggatcgccgcccgagaagctgaagcagggggcatggcccgcacgcgtacgggtccagcgtcagggacatggcgtggttggcctcgcccagaccgtcgcgaaacttgaagttcctcccctccaccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaacttgggatggcgcgcccggaggcgcatgagcgtcgtgtgtacgcaggcgtggcgcgtgtcgaaggtgcacaggttgcagggcacgtcggtctggttggagtccgcgacgtatcgaaacacgtccatctcctggcgcccgacgatcacgccgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccggaccggcgtcgtccacctcggtgagatggagggccgagttggtgctaaataccatggcccccacgagtcccgcggcgcgcgccaggtacgccccgacggcgttggcgcgggccgcggccgtgtcctggccctcgcacagcggccatgcggagatgtcggtgggcggctcgtcgaagacggccatcgacacgatagactcgagggccagggcggcgtctccggccatgacggaggccaggcgctgttcgaacccgcccgccgggcccttgccgccgccgtcgcgcccaccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggttgagggggcggttaaaaaacggaagcgggtaggtaaaattctccccgatcgatcggtggttggcgttgaacggctcggcgatgacccggctaaaatccggcatgaacagctgcaacggatacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcagagtcgcgtcaggtttggggcctgggtgttggggtccaggtgccggccgccgtgaaagacgtacacggacgagctgtagtgcgatggcgtcagtttcagggacaccgcggtacccccgagccccgtcgtgcgagaacccacgaccacggctacgttggcctcaaagccgctctccacggtcaggcccacgaccaggggcgccacggcgacgtcggcatcgccgctgcgcgccgacagtaacgccagaagctcgatgccttcggatggacacgcgcgagcgtacacgtatcccaggggcccgggggggaccttgatggtggttgccgtcttgggctttgtctccatgtcctcctggcaatcggtccgcaaacggaggtaatcccggcacgacgacggacgcccgacgaggtatgtctcccgagcgtcaaaatccggggggggcggcgacggtcaaggggagggtgggagaccggggttggggaatgaatccctacccttcaccgacaacccccgggtaaccacggggtgccgatgaaccccggcggctggcaacgcggggtccctgcgagaggcacagatgcttacggtcaggtgctccgggccgggtgcgtctgatatgcggttggtatatgtacactttacctgggggcgtgccggaccgcaccagcccctcccacaccccgcgcgtcatcagccggtgggcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttttataatagcggccacgcccaccggctacgtcacgctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccatcccacccaagcggcttttggggtttttcctcttcccccctccccacctcccccctctttaggggttcgggtgggaacaaccgcgatgttttccggtggcggcggcccgctgtcccccggaggaaagtcggcggccagggcggcgtccgggttttttgcgcccgccggccctcgcggagccggccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcgaccccaccgtcaccgtctttcacgtgtatgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggttgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgcgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccgcgggccccgatggccttcgggacatccagcgatgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggtcatccagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggattataaccgacaagatcaagctctcgagctacaagctcaacgccgtggccgaagccgtcctgaaggacaagaagaaggacctgagctatcgcgacatccccgcctactacgccgccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgagaacgaacgcgaggagggcgggggcgagcgggagccggagggcgcgcgggagaccgccggccggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgagatgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcatgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatatttgtgctgtgccgcggcctcacggccgccgggctgacggccatgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtatccggagcggccgccgcgttagccgagcgccccgcagaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgccccccccgcggccctgccctccccggccaagcgcccccgggagacgccgtcgcatgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccgggccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaacctgtaccccgggcacgcggtgagcatccggtctatgagccggtagtgcaggtgggcggacgtgccgggaaagatgacgtacagcatgtggcccccgtaagtggggtccgggtaaaacaacagccgcgggtcgcacgccccgcctccgcgcaggatcgtgtggacgaaaaaaagctcgggttggccaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgacacgggagtggtcttcaccaggggcaccgagagcgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcaccacgcccagagtcccgccggccgcagaggagcgcgaccgccggcgctccttgccgtgatagggcccgggccgggagccgcggcgatgggggtcggtatcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaacgctatgcagacacgcccctcgaactcgttcccgcaggccagcttggccttgtggagcagcagctcgtcgggatgggtggcggggggatggccgaacagaacccaggggtcaacctccatctccgtgatggcgcacatggggtcacagaacatgtgcttaaagatggcctcgggccccgcggcccgcagcaggctcacaaaccggcccccgtccccgggctgcgtctcggggtccgcctcgagctggtcgacgacgggtacgatacagtcgaagaggctcgtgttgttttccgagtagcggaccacggaggcccggagtctgcgcagggccagccagtaagcccgcaccagtaacaggttacacagcaggcattctccgccggtgcgcccgcgcccccggccgtgtttcagcacggtggccatcagagggcccaggtcgaggtcgggctgggcatcgggttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgaggagcagcaggaccagatccgcgtacgcccacgtatccggcgactcctccggctcgcggtccccggcgaccgtctcgaattccccgttgcgagcggcggcgcgcgtacagcagctgtccccgcccccgcgccgaccctccgtgcagtccaggagacgggcgcaatccttccagttcatcagtgcggtggtaagcgacggctgcgtgccggataccgccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactgaggcgagaggcggtaggtttgcttgtacagctcgatggtgcggcagaccagacagggccggtccagcgcgaaggtgtcgatggccgccgcggaaaagggcccggtgtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgtcaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtctcgcgagacggcgcgcatgacgcggccgtctggttcgaggatatgacccccgccgagctggaggttgtcttcccgactacggacgccaagctaaactacctgtcgcggacgcagcggctggcctccctcctgacgtacgccgggcctataaaagcgcccgacgacgccgccgccccgcagaccccggacaccgcgtgtgtgcacggcgagctgctcgcccgcaagcgggaaagattcgcggcggtcattaaccggttcctggacctgcaccagattctgcggggctgacgcgcgcgctgttgggcgggacggttcgcgaaccctttggtgggtttacgcgggcacgcacgctcccatcgcgggcgccatggcgggactgggcaagccctacaccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctggggtccaccgagacgacggggttgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggccggaacgggtttccgggccaggggggacggttttggcccgctccccttgttaacccaagggccctcccgcccgtggcaccaggccctgcggggtcttaagcacctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgaccgacacccccatatcgctccccgacctccggtcccgatggccgtcccgcaatttcaccgccccagcaccgttaccaccgatagcgtccgggcgcttggcatgcgcgggctcgtcttggccaccaataactctcagtttatcatggataacaaccacccacacccccagggcacccaaggggccgtgcgggagtttctccgcggtcaggcggcggcactgacggaccttggtctggcccacgcaaacaacacgtttaccccgcagcctatgttcgcgggcgacgcaccggccgcctggttgcggcccgcgtttggcctgcggcgcacctattcaccttttgtcgttcgagaaccttcgacgcccgggaccccgtgaggcccagggagttccttctggggtgttttaatcaataaaagaccacaccaacgcacgagccttgcgtttaatgtcgtgtttattcaagggagtgggatagggttcgacggttcgaaacttaacacaccaaataatcgagcgcgtctagcccagtaacatgcgcacgtgatgtaggctggtcagcacggcgtcgctgtgatgaagcagcgcccggcgggtccgctgtaactgctgttgtaggcggtaacaggcgcggatcagcaccgccagggcgctacgaccggtgcgttgcacgtagcgtcgcgacagaactgcgtttgccgatacgggcggggggccgaattgtaagcgcgtcacctcttgggagtcatcggcgtataacgcactgaatggttcgttggttatgggggagtgtggttccccagggagtgggtcgagcgcctcggcctcggaatccgagaggaacaacgaggtggcgtcggagtcttcgtcgtcagagacatacagggtctgaagcagcgacacgggcgtgggggtagcgtcgatgtgtagcgcgagggaggatgcccacgaagacaccccagacaaggagctgcccgtgcgtggatttgtggaagacgcggaagccgggacggatgggcggttttgcggtgcccggaaccgaaccgccggatactccccgggtgctacatgcccgttttggggctggggttggggctggggcgcggacaggcggctgacggtcaaatgcccccgggggcgcgcagatgtggcgggcgtggccaccggctgccgtgtagtggggcggcgggaaaccgggcctccgggcgtaacaccgccctccagcgtcaagtatgtggggggcgggcctgacgtcgggggcggggtgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcggactggggaccgagcgggaggggcacaccagacaccaaagcgtggggcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggcagaagcgcgggaggaggcgggggggcggggggcggtggcatcggggggcgccggggaactttggggggacggcaagcgccggacgtcgtcgcgggggcccacgggcgccggccgcgtgctttcggccgggacgcccggtcgtgcttcgcgagccgtgactgccggcccagggggccgcggtgcacactgggatgtggggacggactgatcggcggtgggcgaaagggggtccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgtcccaacaaggtccgccgggccgggggtcgggttcgggaccgaggggctctggtcgtcgggggcgcgctggtacaccggatgccccgggaatagctcccccgacaggagggaggcgtcgaacggccgcccgaggatagctcgtgcgaggaaggggtcctcgtcggtggcgctggcggcgaggacgtcctcgccgcccgccacaaacgggagctcctcggtggcctcgctgccaacaaaccgcacgtcgggggggccggggggtccgggttttcccacaacaccgcgaccggggtcatggagatgtccacgagcaccagacacggcgggccccgggcgaggggccgctcggcgatgagcgcggacaggcgcgggagctgtgccgccagacacgcgttttcgatcgggttcaggtcggcgtgcaggaggcggacggcccacgtctcgatgtcggacgacacggcatcgcgcaaggcggcgtccggcccgcgagcgcgtgagtcaaacagcgtgaggcacagctccagctccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatactctgtgtagtcgtccggaaacatgcaggtccaaagagcggccagcgcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggccgaaaacgctggcagcacgcggatgcgggcgtctggatgcggggtcaggcgctgcacgaatagcatggaatctgctgcgttctgaaacgcacgggggagggtgagatgcatgtactcgtgttggcggaccagatccaggcgccaaaaggtgtaaatgtgttccggggagctggccaccagcgccaccagcacgtcgttctcgttaaaggaaacgcggtgcctagtggagctctggggtccgagcggcggccccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgaggacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggccgatccagcgtactggtggcctcgcgcagcaccaggggccccgggcctccgctcactcgcaggtacgcctcgccccggcggcgcagcatctgcgggtcggcctcttggccgggtggggcggacgcccgggcgcgggcgtctagggcgcgaagatccacgagcaggggcgcgggcgcggcggccgcgcccgcgcccgtctggcctgtggccttggcgtacgcgctatataagcccatgcggcgttggatgagctcccgcgcgccccggaactcctccatcgcccatggggccaggtccccggccaccgcgtcgaattccgccaacaggccccccagggtatcaaagttcatctcccaggccacccttggcaccacctcgtcccgcagccgggcgctcaggtcggcgtgttgggccacgcgccccccgagctcctccacggccccggcccgctcggcgctcttggcgcccaggacgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgagagacctccgcagggcctcgacgcggcgacgggtgtcgaagagcgcctgcaggcgcgcgccctgtcgcgtcaggaggcccgggccgtcgctgctggccgcgcttagcgggtgcgtctcaaaggtacgctgggcatgttccaaccaggcgaccgcctgcacgtcgagctcgcgcgccttctccgtctggtccaacagaatttcgacctgatccgcgatctcctccgccgagcgcgcctggtccagcgtcttggccacggtcgccgggacggcgaccaccttcagcagggtcttcagattggccagaccctcggcctcgagctgggcccggcgctcgcgcgcggccagcacatcccgcagccccgccgtgacccgctcggtggcttcggcgcgctgctgtttggcgcgcaccacggcgtccttggtatcggccaggtcctgtcgggtcacgaatgcgacgtagtcggagtacgccgtgtccttcacggggctctggtccacgcgctccagcgccgccacgcacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtagtacggcctcgtggtagtcgataaacccgtcgttcgcctgggcccgctccagcagcccccccgcgagccgcagaagccgcgccaggggctcggtgtccacccgaaacatgtcggcgtacgtgtcggccgcggccccaaaggccgcgctccagtcgatgcggtgaatggctgcgagcggggggagcatggggtggcgctggttctcgggggtgtatgggttaaacgcaagggccgtctccagggcaagggtcaccgccttggcgttggttcccagcgcctgctcggcccgctttcggaagtcccgggggttgtagccgtgcgtgcccgccagcgcctgcaggcgacggagctcgaccacgtcaaactcggcactgctttccacgcggtccagcacggcctccacgtcggcggcccagcgctcgtggctactgcgggcgcgctgggccgccatcttctctctgaggtcggcggtggcggcctcaagttcgtcggcgcggcgtcgcgtggcgccgatgacctttcccagctcctgcagggcgcgcccgctgggggagtggtccccggccgtcccttcggcgtgcaacaggcccccgaacctgccctcgtggcccgcgaggctttcccgcgcgccggtggtcgcgcgcgtcgcggcttggatcagggaggcatgctctcccttcggttggttggcggcccggcgcacctggacgacaaggtcggcggcagccgaccctaaggtcgtgagctgggcgatggccccccgcgcgtccagggccaaccgagtcgccttgacgtatcccgcggcgctgtcggccatggccgctaggaaggccaggggggaggccgggtcgctggcggccgcgcccagggccgtcaccgcgtcgaccaggacgcggtgcgcccgcacggccgcatccaccgtcgacgcggggtctgccgtcgcgacggcggcgctgccggcgttgatggcgttcgagacggcgtgggctatgatcggggcgtgatcggcgaagaactgcaagagaaacggagtctcgggggcgtcggcgaacaggttcttcagcaccaccacgaagctgggatgcaagccggacagagccgtcgccgtgtccggagtcgggtgctccagggcatctcggtactgccccagcagcccccacatgtccgcccgcagcgccgccgtaacctcagggggcgccccccgaacggcctcggggaggtccgaccagcccgccggcagggaggcccgcagggtcgccaggacggccggacaggcctttagccccacaaagtcagggagggggcgcaggaccccctggagtttgtgcaagaacttctcccgggcgtcgcgggccaccttcgcccgctcccgcgctccctcgagcattgcctccagggagcgcgcgcgctcccgcaaacggacacgcgcatcgggggcgagctctgccgtcagcttggcggcatccatggcccgcgcctgccgcagcgcttcctcggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgctggtcgttaatgtcgcggatgctgcgcgccagctcgtccagcggcttgcgttctatcagcccttggttggcggcgtccgtcaggacggagagccaggccgccaggtcctcgggggcgtccagcgtctggccccgctgtatcagatcccgcaacaggatggccgtggggctggtcgcgatcgggggcggggcgggaatggcggcgcgctgcgcgatgtcccgcgtgtgctggtcgaagacaggcagggactcgagcagctggaccacgggcacgacggcggccgaagccacgtgaaaccggcggtcgttgttgtcgctggcctgtagagccttggcgctgtatacggccccccggtaaaagtactccttaaccgccccctcgatcgcccgacgggcctgggtccgcacctcctccagccgaacctgaacggcctcggggcccagggggggtgggcgcggagccccctgcggggccgccccggccggggcgggcattacgccgaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcgtcggggtggcgggggcaaaaagggtgtccgggtgggcctgcgagcgctccagaagccactcgccgaggcgtgtatacagattggccggcggggccgcgcgaagctgcagctccaggtccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccggggcagcatgctcagggtaaactccaacagggcggcggccgggccggccaccccggcctgggtgtgcgtccgggccccgttctcgatgagaaaggcgaggacgcgttcaaagaaaaaaataacacagagctccagcagccccggagaagccggatacggcgaccgtaaggcgctgatggtgagccgcgaacacgcggcgacctcgcgggccagggcggcggagcacgcggtgaacttaaccgccgtggcggccacgtttgggtgggcctcgaacagctgggcaaggtctgcgcccgggggctcgggtgagcggcgagtcttcagcgcctcgagggcctgcgaggacgccggaaccgtgggcccgtcgtcctcgcccgcctcggcgaccggcggcccggccgggtcggggggtgccgaggcgaggacaggctccggaacggaggcggggaccgcggccccgacgggggttttgcctttgggggtggatttcttcttggttttggcagggggggccgagcgtttcgttttctcccccgaagtcaggtcttcgacgctggaaggcggagtccaggtgggtcggcggcgcttgggatggccggccgagtagcgtgcccggtgccgaccaaccgggacgacgcccatctccaggacccgcatgtcgtcgtcatcttcttcggccgcctctgcggcggggggcttgggggcggagggaggcggtggtgggatcgcggagggtgggtcggcggaggggggatccgtgggtggggtacccttcagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtgtggccccgtgccccctaccccctcccgggggcccacgccgacgcagggctcccccaggcccgcgatctcgccccgcagggggtgcgtgatggccacgcgccgttcgctgaacgcttcgtcctgcaggtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccgtgcgggtcaaaaaggtatgcggtgtcgccgtccctgaacagccccatccctagggggccaatggttaggagcgtgtacgacagggggcgcagggcccacgggccggcgaagaacgtgtgtgcggggcattgtgtctccagcaggcccgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcggtcgcgcgcgtccactcgcccccctcgaccaaacaaccctcgatggcctccgcggacagaacgtcgcgagggcccacatcaaatatgaggctgagaaaggacagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatatgtgccctactggccagagacccggcctgtttatggaccggacccccggggttagtgttgtttccgccacccatgcccccgtaccatggccccggttcccctgattaggctacgagtcgcggtgatcgcttcccaaaaaccgagctgcgtttgtctgtcttgatcttcccccccccccccgcccgcccgcccgcccgcccgcccgcacaccataacaccgagaacaacacacgggggtgggcgtaacataataaagctttattggtaactagttaacggcaagtccgtgggtggcgcgacggtgtcctccgggctcatctcgtcgtcctcgacgggggtgttggaatgaggcgccccctcgcggtccgcctggcgtgggccgtgcccataggcctccggcttctgtgcgtccatgggcataggcgcggggagactgtttccggcgtcgcggacctccaggtccctgggagactccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgagagacgccgccagcgcgctagaaactgacccatgaagcaaaacccggggacctggtctcccgacagcagcttcgacgcccgggtgtgaatgccggacacaacggacagaaacccgtgaatttcgcgccggaccacggccagcacgttgtcctcgtgcgacacctgggccgccagctcgtcgcacacctccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggagtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtctgcgcgctcggcgaggtcctgcagcaccccccgggcggccagggcgtacatgctaatcaacaggaggctggtgcccacctcggggggcggggggggctgcagctggaccaggggccgcagctgctcgacggcacccctggagatcacgtacagctcccggagcagctgctctatgttgtcggccatctgcatagtggggccgaggccgccccgggcggccggttcgaggagggtaatcagcgcgcccagtttggtgcgatggccctcgaccgtggggagatagcccagcccaaagtcccgggcccaggccaacacacgcagggcgaactcgaccgggcgtggaaggtaggccgcgctacacgtggcccccaacgcgtccccgaccaccagggccagaacgtaggggacgaagcccgggtcggcgaggacgttggggtgaatgccctcgagggcggggaagcggatctgggtcgccgcggccaggtggacagagggggcgtggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggacccatgatacgcgccagtgtctggctcagaacgccccccgacaggccgaccgcctcgcagagccgcccgtgcgtgtgctcgctggcgccctggacccgcctgaaagtttttacgtagttggcatagtacccgtattcccgcgccagaccaaacacgttcgaccccgcgagggcaatgcacccaaagagctgctggacttcgccgagtccgtggccggcgggcgtccgcgcggggacgcccgccgccagaaacccctccagggccgaaaggtagtgcgtgcagtgcgagggcgtgaacccagcgtcgatcagggtgttgatcaccacggagggcgaattggtattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctccagtggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactcaccagggccaagaaccggaggccctcttgcacgaacggggcggggaagagcaggctgtacgccggggtggtaaggttcgcgctgggctgccccaacgggaccggcgccatcttgagtgacgtctccccaagggcctcgatggaggtccgcgggctcatggccaagcagctcttggtgacggtttgccagcggtctatccactccacggcgcactggcggacgcggaccggccccagggccgccgcggtgcgcaggccggcggaatccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcatgcccgcgcgtctaccggggcaatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggtgggggacagcgtagaccaacgacgagatcgggcgggaatgactgtcgtgcgctgtagggagcggcgaattatcgatcccccgcggccctccaggaaccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccctcccccgggttttgttaggtcgcgatctgcagtcgcaatgaagaccaatccgctacccgcaaccccttccgtgtggggcgggagtaccgtggaactcccccccaccacacgcgataccgcgggacagggcctgcttcggcgcgtcctgcgccccccgatctctcgccgcgacggcccagtgctccccagggggtcgggaccccggagggcggccagcacgctgtggttgcttggcctggacggcacagacgcgccccctggggcgctgacccccaacgacgataccgaacaggccctggacaagatcctgcggggcaccatgcgcgggggggcggccctgatctgctccccgcgccatcatctaacccgccaagtgatcctgacggatctgtgccaacccaacgcggatcgtgccgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctcgtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccacaggaggcggcgcacaccggccacccgggccggccccgctcggccgtgatcctcccggcgtgtgcgttcgtggacctggacgccgagctggggctggggggcccgggtgcggcgtttctgtacctggtattcacttaccgccagcgccgggaccaggagctgtgttgtgtgtacgtgatcaagagccagctccccccgcgcgggttggagccggccctggagcggctgtttgggcgcctccggatcaccaacacgattcacggcaccgaggacatgacgcccccggccccaaaccgaaaccccgacttccccctcgcgggcctggccgccaatccccaaaccccgcgttgctcggctggccaggtcacgaacccccagttcgccgacaggctgtaccgctggcagccggacctgcgggggcgccccaccgcacgcacctgtacgtacgccgcctttgcagagctcggcatgatgcccgaggatagtccccgctgcctgcaccgcaccgagcgctttggggcggtcagcgtccccgttgtcatcctggaaggcgtggtgtggcgccccggcgagtggcgggcatgcgcgtgagcgtagcaaacgccccgcccacacaacgctccgcccccaaccccttccccgctgtcactcgtggttcgttgacccggacgtccgccaaataaagccactgaaacccgaaacgcgagtgttgtaacgtcctttgggcgggaggaagccacaaaatgcaaatgggatacatggaaggaacacacccccgtgactcaggacatcggcgtgtccttttgggtttcactgaaactggcccgcgccccacccctgcgcgatgtggataaaaagccagcgcgggtggtttagggtaccacaggtgggtgctttggaaacttgtcggtcgccgtgctcctgtgagcttgcgtccctccccggtttcctttgcgctcccgccttccggacctgctcttgcctatcttctttggctctcggtgcgattcgtcaggcagcggccttgtcgaatctcgaccccaccactcgccggacccgccgacgtcccctctcgagcccaccgaaacccgccgcgtctgttgaaatggccagccgcccagccgcatcctctcccgtcgaagcgcgggccccggttgggggacaggaggccggcggccccagcgcagccacccagggggaggccgccggggcccctctcgcccacggccaccacgtgtactgccagcgagtcaatggcgtgatggtgctttccgacaagacgcccgggtccgcgtcctaccgcatcagcgatagcaactttgtccaatgtggttccaactgcaccatgatcatcgacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacggccgacgtccccaccgaggcccttgggggcccccctcctcctccccgcttcaccctgggtggcggctgttgttcctgtcgcgacacacggcgccgctctgcggtattcgggggggagggggatccagtcggccccgcggagttcgtctcggacgaccggtcgtccgattccgactcggatgactcggaggacacggactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcgccgacgccagaggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattcggcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggattttggcctgacggaaaggcttcgcgagcacgaagcccagctggtgatcctggcccaggctctggaccattacgactgtctgatccacagcacaccacacacgctggtcgagcgggggctgcaatcggccctgaagtatgaggagttttacctaaagcgttttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtccttgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagcgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgttgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcacgagctgcgcgctgtgaccgacaaaccccctccgcgccaggcccgccgccactgtcgtcgccgtcccacgctctcccctgctgccatggattccgcggccccagccctctcccccgctctgacggcccttacgggccagagcgcgacggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgcttttccacaacaacgaccaggcgcgccgcgagtacgtggccggcaccatcaaccacccggccatccgcgccaaggtggactggttggaagcgcgggtgcgggaatgcgcctccgttccggaaaagttcattctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagtgtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccgtccagattctgcgggcatagaataccataattttattggtgggtcgtttgttcggggacaagcgcgctcgtctgacgtttgggctactcgtcccagaatttggccaggacgtccttgtagaacgcgggtgggggggcctgggtccgcaactgctccagaaacctgtcggcgatatcaggggccgtgatatgccgggtcacgatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgccggttggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcgtctcggtgccgccggcggccggcggcagagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtacttcagtatatcgcgacagttgatcgtgggaatgtagcaggcgctaatatccaacacaatatcacagcccatcaacaggaggtcagtgtccgtggtgtacacgtacgcgaccgtgttggtgtgatagaggttggcgcaggcatcgtccgcctccaactgacccgagttaatgtaggcgtaccccagggcccggagaacgcgaatacagaacagatgcgccagacgcagggccggcttcgagggcgcggcggacggcagcgcggctccggacccggccgtcccccgggtccccgaggccagagaggtgccgcgtcggcgcatgttggaaaaggcagagctgggtctggagtcggtgatgggggaaggcggtggagaggcgtccacgtcactggcctcctcgtccgtccggcactgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtatcggcgctgatatttgaccaccaacgtgtacatgacgttccacaggtccacggcaatgggggtgaagtacccggccggggccccaaggccccggcgcttgaccagatggtgtgtgtgggcaaacttcatcatcccgaacaaacccatgtcaggtcgattgtaactgcggatcggcctaactaaggcgtggttggtgcgacggtccgggacacccgagcctgtctctctgtgtatggtgacccagacaacaacaccgacacaagaggacaataatccgttaggggacgctctttataatttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttgggatgacggattcccctggcggtgtggcccccgcctcccacgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaaatttccgtgttcaccacgagtacctgcgtcacatttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggtcgtcgacgattgcagcatgcgggcggtgctccggcgactgcaggtcggcgggggcaccctcaagttcttcctcacgacccccgtccccagtctgtgcgtcaccgccaccggtcccaacgctgtatcggcggtatttctcctgaaaccccagaagatttgcctggactggctgggtcatagccaggggtctccttcagccgggagctcggcctcccgggcctctgggagcgagccaacagacagcaaggactccgcgtcggacgcggtcagccacggcgatccggaagacctcgatggcgctgcccgggcgggagaggcgggggcctcgcacgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccgcagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccggggggggccccaaaccccgtatggttttggattcccctgacggggcggggccttggcggccgcccaactctcgcaccatcccgggttaatgtaaataaacttggtattgcccaacactctcccgcgtgtcgcgtgtggttcatgtgtgtgcctggcgtcccccaccctcgggttcgtgtatttcctttccctgtccttataaaagccgtatgtggggcgctgacggaaccaccccgcgtgccatcacggccaaggcgcgggatgctccgcaacgacagccaccgggccgcgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggcacgcagccgggggccccggccccgggcccggccacccagtgcctggcgctgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccttggccgcggcggtgttggcgggcccgggggcgaccaccgccagggactgcttctccagggcgtgccccgaccaccgccgcgtctgcgtcatcgtcgcaggcgagtctgtttcccgccgccccccggaggacccagagcgacccggggaccccgggccaccgtcccccccgacaccccaacgatcccaggggccgccggccgatgaggtcgcaccggccggggtagcgcggcccgaaaacgtctgggtgcccgtggtcacctttctgggggcgggcgcgctcgccgtcaagacggtgcgagaacatgcccggggaacgccgggcccgggcctgccgctgtggccccaggtgtttctcggaggccatgtggcggtggccctgacggagctgtgtcaggcgcttgcgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcgcaggaaggaccccggggacggggcccggttggcggcgacgcttcggggcctcttcttctccgtgtacgcgctggggtttggggtgggggcgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccctcactaccgagggcgcttggtcgggaggccgcatcgaacgcacacccccatccggtggtccgtgtggaggtcgttttcagtgcccggtctcgctttgccgggaacgctagccgatccctcgcgagggggaggcgtagggcatggccccggggcgggtgggccttgccgtggtcctgtggagcctgttgtggctcggggcgggggtgtccgggggctcggaaactgcctccaccgggcccacgatcaccgcgggagcggtgacgaacgcgagcgaggcccccacatcggggtcccccgggtcagccgccagcccggaagtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctgacggacccccacgtgctctgggcggagggggccggcccgggtgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgacgcctactacccgcgtaaccccgtggagttggtctggttcgaggacgaccgccaggtgtttaacccgggccagatcgacacgcagacgcacgagcaccccgacgggttcaccaccgtctctaccgtgacctccgaggctgtcggcggccaggtccccccgcggaccttcacctgccagatgacgtggcaccgcgactccgtgacattctcgcgacgcaatgccaccgggctggccctggtgctgccgcggccaaccatcaccatggaatttggggtccggcatgtggtctgcacggccggctgcgtccccgagggcgtgacgtttgcctggttcctgggggacgacccctcaccggcggctaagtcggccgttacggcccaggagtcatgcgaccaccccgggctggctacggtccggtccaccctgcccatttcgtacgactacagcgagtacatctgtcggttgaccggatatccggccgggattcccgttctagaacaccacggcagtcaccagcccccacccagggaccccaccgagcggcaggtgatcgaggcgatcgagtgggtggggattggaatcggggttctcgcggcgggggtcctggtcgtaacggcgatcgtgtacgtcgtccgcacatcacagtcgcggcagcgtcatcggcggtaacgcgagacccccccgttacctttttaatatctatatagtttggtcccccctctatcccgcccaccgctgggcgctataaagccgccaccctctcttccctcaggtcatccttggtcgatcccgaacgacacacggcgtggagcaaaacgcctccccctgagccgctttcctaccaacacaccggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgcccttcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggccaacgagggttggggggtccggcacggacccaaaataataaacacacaatcacgtgcgataaaaagaacacgcggtcccctgtggtgtttttggttatttttattaaatctcgtcgacaaacagggggaaaggggcgtggtctagcgacggcagcacgggcggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggcccggtgcggcccgggggggaaaatagggcggatcccccccagtcgtacaggggattttccgcctcaatgtacggggaggccggcgctgcattcgccgtgttcacgcagacgttttcgtagacccgcatccatggtatttcctcgtagacacgccccccgtcctcgcgcaccgtctcgtatattgactcgtcgtcctcgtagggggcgtgccgttcgcgggccgaggcggcgtgggtggctttgcggcgggcgtcgtcgtcgtcgtcgtcggccgtcagatacgtggcttccatctggtcgggttctccctccggggcgggtccccacacccgtggccgatcgaggctccccagagacgcgcgccggacgaggagggggcacgtcgccgccggcggtcgcctgtcgggtcccgcgacgttacgggccgggaggcgcgggggcacctcccccatgtgcgtgtaatacgtggccggctgtgcggccgcagcggggggctcggcgaccgggtcgttcgcatccggaagcgggggccccgcgccgtccgcgcggcgcctccggaacctccgggtggacgcgggggtcgagtgtaggcgaggtcgggggaggggcgggggctcgttgtcgcgccgcgcccgctgaatcttttcccgacaggtcccaccccccgcgcgatgcccccccgggccgctggccatgtcgtccgggggaggccccgcggaccacgtcgtccggcgagacgccacgagccgcaggatggactcgtagtggagcgacggcgccccgctgcggagcagatccgcggccagggcggccccgaaccaagccttgatgctcaactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggatacgacggtatccacggtcccgtacgtaccgccgctccgcttgccggtctgccacaggttggctaggcgcgtcaggtggcccaggacgtcgctgaccgccgccctgagcgccatgcactgcatggagccggttgtgccgctgggaccccggtccagatggcgcgcgaacgtttccgcgggcgcctccgggctgccgccgagcgggaggaaccggcgattggagggactcagccggtgacatacgtgcttgtctgtcgtccacagcatccaggacgcccaccggtacagcacggagacgtaggccaggagctcgttgagccgcagtgcggtgtcggtgctggggcggcttgggtccgccgggcgcataaagaacatgtactgctgaatccgatggagggcgtcgcgcaggccggccacggtggcggcgtacttggccgccacggccccgctcttgaacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggcgcggctcaactggccgccgggcagctccacctcgcccagcgcctgggtggcggccgaagcgtagtgccggatgtactcgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgtccaccacgcggacccgcgggcccaacacggacccgccgcttacgggggcgggggggaagggctccaggtccttgagaagaaaggcggggtctgccgtcccggacacgggggcccggggcgctgaggaggcggggcgcagatccacgtgctccgcggccgcgcggacgtccgcccagaacttggcgggggtggtgcgcgcgtacaggggctgggtcgctcggaggacgcacgcgtagcgcaggggggtgtacgtgcccacctcgggggccgtgaatcccccgtcaaacgcggccagtgtcacgcacgccaccacggtgtcggcaaagcccagcagccgctgcaggacgagcccggcggccagaatggcgcgcgtggccgccgcgtcgtcccggcgccggtgcgcgtccccgcacgcccgggcgtactttaaggtcacggtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccagcacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcggaacaccgcggcgtcggggccggattggggggacactaacccccccgcgctcagtaacggcacggccgcggccccgacgggacgcaacgccgtgaggctcgcgaactgccgcctcagctcggccgccctgtcgtccaggtcagacccgcgcgcctccgcgtgaaggcgcgtcccgcacacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggactcgtcccacacggcaacctgaggggccagtcgatccaacgggaatgccgcccggagctccgggcccggcacgcgtccctccagaacctccaccttgggcggggaacgggccccgccgccgtcctccggcccgacggcttccgggtagtcgtcctcctcgtactgcagctcctctaggaacagcggcgacggcgccacccgcgaaccgccgacccgccccaaaatagcccgcgcgtcgacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgtgggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccaccccatcgcccgctggctcgtccgccacgggcgaggcgcgggggcgggtggatgcgcgcctcctgcgccccgcgggttcgcgagccgacatggtggcgatagacgcgggttatcggatgtccgctaccccccaaaaaagaaaaagaccccacagcgcggatggaggccggggtaggtgccgccggaccccctcgcgatgggaatggacgggagcgacggggccggcgcaaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttctttcccccaccaccaaataatcagacgacaaccgcaggttttgtaatgtatgtgctcgtgtttattgtggatacgaaccggtgacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggccccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctgggtgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacagaacacgcgccagacgagcggtctctcggtagtacctgtccgcgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtcgggaaggccacgtcgccgtgggcgcgaatgtcgatttgggcgcgttcggggacgtacgcgtccccccattccaccacatcgctgggcagcgttgataggaatttacactcccggtacaggtcggcgttggtcggtagcgccgaaaacagatcctcgttccaggtatcgagcatggtacatagcgcggggcccgcgctaaagcccaagtcgtcgaggagacggttaaagagggcggcgggggggacgggcatgggtggggagggcatgagctgggcctggctcaggcgccccgttgcgtacagcgggggggccgccggggtgtttttgggacccccggctgggcgggggggcggtggcgaagcgccgtccgcgttcatgtcggcaaacagctcgtcgaccaagaggtccattgggtggggttgatacgggaaagacgatatcgggcttttgatgcgatcgtccccgcccgcccagagagtgtgggacgcccgacggcgcgggaagagaaaaacccccaaacgcgttagaggaccggacggaccttatggggggaagtgggcagcgggaaccccgtccgttcccgaggaatgacagcccgtggtcgccaccacgcatttaagcaacccgcacgggccgccccgtacctcgtgacttccccccacattggctcctgtcacgtgaaggcgaaccgagggcggctgtccaacccaccccccgccacccagtcccggtccccgtcggattgggaaacaaaggcacgcaacgccaacaccgaatgaacccctgttggtgctttattgtctgggtacggaagttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaagcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccgaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtgtgcgtccggcccctccggacccagcgggtggcggaggtgcccgcgcaggccccgggccggacaaaaccgccccggaaacgggacgccgcgtccgggggacctccgggtgttcgtcgtcttcggatgacgagcccccgtagagggcataatccgactcgtcgtactggacgaaacggacctcgcccctctggcgcgagcgtgtctgtagggcgccacggcgggaggtgtcaggcggactatcgggactcgccatacctgaagacggggtgtagtacagatcctcgtactcatcgcgcggaacctcccgcggacccgacttcacggagcggcgagaggtcatggttccacgaacacgctagggtcggatgcgcggacaattaggcctgggttcggacggcgggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaggaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagtgttttgggtctcgcaccgcgcgcccccgatcccatcgcgcccgccctcctcgccgggcggctccccgtgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggttagcggggactccggatgggccgtcgggcgcgtctctctggacctgcgaatggctatgccggctgacttttgcgcgattattcacgcccccgcgctagccagccccgggcaccacgtaatactgggtcttatcgactcggggtaccgcggaaccgttatggccgtggtcgtagcgcctaaaaggacgcgggaatttgcccccgggaccctgtgggtcgacgtgacgttcctggacatcctggcgacccccccggccctcaccgagccgatttccctgcggcagttcccgcaactggcgcccccccctccaaccggggccgggatacgcgaagatccttggttggagggggcgctcggggccccaagcgtgactacggccctaccggcgcgacgccgagggcggtccctcgtctatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgctatgtgttggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcctggtgtttacgaacgagtttgacgcggaggcccccccgagcgagcgcgggaccgggggttttggctctaccggtatttagcccacagctttgggttcgttccgggcaataaaaaacgtttgtatcgcatctttcctgtgtgtagttgtttatgttggatgcctgtgggtctatcacacccgcccctccatcccacaaacacaaaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaatcccccccgaccgggttcccccggcaaccccgaaggcggagaggccaagggcccgttcggcgatggccacatcctccatgaccacgtcactctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgagcccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccgccagggcctcctgcagccgcggctccgcctccgagggcggaacggccgcgcggatcatctcatattgttcctcggggcgcgctccccagccacatatagccccgagaagagaagccatcgcgggcgggtactggcccttgggcgcgcggacgcaatggggcaggaagacgggaaccgcggggagaggcgggcggccgggactcccgtggaggtgaccgcgctttatgcgaccgacgggtgcgttattacctcttcgatcgccctcctcacaaactctctactgggggccgagccggtttatatattcagctacgacgcatacacgcacgatggccgtgctgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggcgcgcgtgcttgccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgctgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatctacctggcccattttgagtgtttcagccccccgcgcctagccacgcatcttcgggccgtgacgacccacgaccccaaccccgcggccagcacggagcagccctcgcccctgggcagggaggccgtggaacaatttttttgtcacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggacacggcgggcggcacgccgcccccacagacctgcggaattgtcaagcgcctcctgagactggccgccacggaacagcagggcaccacacccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggagtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggagggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggtacgccatgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtctggcgtctgcgggcgctgttgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccggaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcggggcccccgcccggggggcggaacgaggaggggtttgggagagccggccccggcaccacgggtataaggacatccaccacccggccggtggtggtgtgcagccgtgttccaaccacggtcacgcttctgtgcctctccccgattcgggcccggtcgctcgctaccggtgcaccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggcatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgttggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctcaccaacctgttcttacgggccccggacataacccacgttgcccccccttactgcctcaacgccacctggcaggccgaaacggccatgcacaccagcaaaactgactccgcttgcgtggccgtgtggagttacctggtccgcgcctcctgtgagaccagcggcacaatccactgctttttctttgtggtatacaaggacacccaccatacccctccgctgattaccgagctccgcaactttgcggacctggttaaccacccgccggtcctacgcgaactggaggataagcgcggggtgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatttccaaccccccatgaatgtgtgtaaccccccccaaaaaaataaagagccgtaacccaaccaaaccaggcgtggtgtgagtttgtggacccaaagccctcagagacaacgcgacaggccagtatggaccgtgagacttttatttattaactcacaggggcgcttaccgccacaggaataccagaataatgaccaccacaatcgcgaccaccccaaatacagcatggcgccccaccacgccacaacagccctgtcgccggtatggggcatgatcagacgagccgcgagccgcgcgttgggccctgtacagctcgcgcgaattgaccctaggaggccgccacgcgcccgagttttgcgttcgtcgctggtcgtcgggcaccaaagccccggacggctgttcggtcgaacgaacggccacgacagtggcataggttggggggtggtccgacatagcctcggcgtacgtcgggaggcccgacaagaggtcccttgtgatgtcgggtggggccacaagcctggtttccggaagaaacaggggggttgccaataacccgccagggccaaaactccggccctggcgcacgtcgttcggcgcggcgccgggcgcgccgagcggctcgctgggcggcttggcgtgagcggccccgctccgacgcctcgccctctccggaggaggttggtggaattggcacggacgacaggggcccagcagagtacggtggaggtgggtccgtgggggtgtccagatcaataacgacaaacggcccctcgttcctaccagacaagctatcgtaggggggcgggggatcaacaaacgcgttccccgcgctccatagacccgcgtcgggttgcgccgcctccgaagccatggatgcgccccaaagccacgactcccgcgcgctaggtccttggggtaagggaaaaggccctactccccatccaagccagccaagttaacgggctacgccttcggggatgggactggcaccccggcggattttgttgggctggcatgcgtcgcccaaccgagggccgcgtccacgggacgcgccttttataaccccgggggtcattcccaacgatcacatgcaatctaactggctcccctctcctcccctctcccctctcccctctcccctctcccctctcccctctcccctcttaggttggggggtggtccgacatagcctcggcgtacgtcgggaggcccgacaagaggtcccttgtgatgtcgggtggggccacaagcctggtttccggaagaaacaggggggttgccaagcggcccggcccgcgctcccccccccccggggccgtgtccttgctttccccccgtctccccccccctcctcctccttctcctcctcctcgtttttccaaaccccgcccacccggcccggcccggcccggccaccgccgcccacccacccaccgcgggagacccagccccggtcccccgttccccgggggccgttatctccagcgccccgtccggcgcgccgccccccgccgctaaaccccatcccgcccccgggaccccacatataagcccccagccacacgcaagaacagacacgcagaacggctgtgtttatttaaataaaccgatgtcggaataaacaaacacaaacacccgcgacggggggacggagggaggggggtgacgggggacgggaacagacacaaaaaacaaccacaaaaaaacagccacccccgacaccccccaccccagtctcctcgccttttcccacccaccccacgcccccactgagcccggtcgatcgacgagcacccccgcccccgcccctgccccggcgacccccggcccgcacgatcccgacaacaataacaaccccaacggaaagcggcggggtgttgggggaggcgaggaacaaccgaggggaacgggggatggaaggacgggaagtggaagtcctgatacccatcctacacccccctgccttccaccctccggccccccgcgagtccacccgccggccggctaccgagaccgaacacggcggccgccgcagccgccgcagccgccgccgacaccgcagagccggcgcgcgcacacacaagcggcagaggcagaaaggccccgagtcattgtttatgtggccgcgggccagcagacggcccgcgacacccccccgcccgtgtgggtatccggccccccgccccgcgccggtccattaagggcgcgcgtgcccgcgagatatcaatccgttaagtgctctgcagacaggggcaccgcgcccggaaatccattaggccgcagacgaggaaaataaaattacatcacctacccacgtggtgctgtggcctgtttttgctgcgtcatctgagcctttataaaagcgggggcgcggccgtgccgatcgcgggtggtgcgaaagactttccgggcgcgtccgggtgccgcggctctccgggcccccctgcagccggggcggccaaggggcgtcggcgacatcctccccctaagcgccggccggccgctggtctgttttttgttttccccgtttcgggggtggggggggttacggtttctgttttttaaacccgtctggggtgtttttcgttccgtcgccgggatgtttcgttcgttcggcccctcacggggcgaaggccgcgtacggcccgggacgaggggcccccgaccgcggcggtccgggccccgtccgggcccgctcgccggcacgcgacgcgaaaaaggccccccggaggcttttccgggttcccggcccggggcctgagataaacaatcggggttaccgccaacggccggcccccgtggcggcccggcccggggccccggcggacccaaggggccccggcccggggccccacaacggcccggcgcatgcgctgtgttttttttttcctcggtgttctgccgggctccgtcgcctttcctgttctcgcttcttccccccccccttcttcacccccagtaccctcctccctcccttcctcccccgttatcccactcgtcgagggcgccccggtgtcgttcaacaaagacgccgcgtttccaggtaggttagacacctgcttctccccaatagagggggggggacccaaacgacagggggcgccccagaggctaaggtcggccacgccactcgcgggtgggctcgtgttacagcacaccagcccgttcttttccccccctcccacccttagtcagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgcctggggggggggatgcgggggagggggggtgtggaaggaaatgcacggcgcgtgtgtaccccccccccaaagttgttcctaaagcgaggatatggaggagtggcgggtgccgggggacgggggtgatctctggcacgcggggggggaagggtcgggggagggggggatggggtaccggcccacctggccgacgcgggtgcgcgtgcctttgcacaccaaccccacgtcccccggcggtctctaagaagcaccgccccccctccttcataccaccgagcatgcctgggtgtgggttggtaaccaacacgcccatcccctcgtctcctgtgattctctggctgcaccgcattcttgttttctaactatgttcctgtttctgtctcccccccacccctccgccccaccccccaacacccacgtctgtggtgtggccgacccccttttgggcgccccgtcccgccacccctcccgtcctttgttgccctatagtgtagttaaccccccccccgccctttgtggcggccagaggccaggtcagtccgggcgggcaggcgctcgcggaaacttaacacccacacccagcccactgtggttctggctccatgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccaagatccatccaacacagacagggaaaagatacaaaagtaaacctttatttcccaatagacagcaaaaatcccctgagttttttattagggccaacactaaagacccgctggtgtgtggtgcccgtgtctttcactttcccctccccgacacggattggctggtgtagtgggcgcggccagagaccacccagcacccgacccccctccccacaaacacggggggcgtcccttattgttttccctcgtcccgggtcgacgccccctgctccccggaccacgggtgccgagaccgcaggctgcggaagtccagggcgcccactagggtgccctggtcgaacagcatgttccccacgggggtcatccagaggctgttccactccgacgcgggggccgtcgggtactcggggggcatcacgtggttacccgcggtctcggggagcagggtgcggcggctccagccggggaccgcggcccgcagccgggtcgccatgtttcccgtctggtccaccaggaccacgtacgccccgatgttccccgtctccatgtccaggatgggcaggcagtcccccgtgatagtcttgttcacgtaaggcgacagggcgaccacgctagagacccccgagatgggcaggtagcgcgtgaggccgcccgcggggacggccccggaagtctccgcgtggcgcgtcttccgggcacacttcctcggcccccgcggcccagaagcagcgcgggggccgagggaggtttcctcttgtctccctcccagggcaccgacggccccgcccgaggaggcggaagcggaggaggacgcggccccggcggcggaagaggcggcccccgcgggggtcggggccgaggaggaagaggcagaggaggaagaggcggaggccgccgaggacgtcaggggggtcccgggcccaccctggccgcgcccccccggccctgagtcggagggggggtgcgtcgccgccctcttggcccctgccggcgcgagggggggacgcgtggactggggggaggggttttcctggcccgacccgcgcctcttcctcggacgcaccgccgcctcctgctcgacagagacggcggaggggagcggggcggcgccggagggggtgcggccgcgggagggcccgtgcccaccctccacgcccggcccccccgagccgcgcgccaccgtcgcacgcgcccggcacagactctgttcttggttcgcggcctgagccagggacgagtgcgactggggcacacggcgcgcgtccgcggggcgggcggccggctccgccccgggggccggggcgcgggggccgggccccggaggcggcgctcgcacgcacggggccacggccgcgcgggggcgcgcgggtcccgacgcggccgaggacgcggggggcccggggcggggggcggagcctggcatgggcgccgcggggggcctgtggggagaggccgggggggagtcgctgatcactatggggtctctgttgtttgcaaggggggcgggtctgttgacaagggggcccgtccggcccctcggccgccccgcctccgcttcaacaaccccaaccccaaccccaacccccccggaggggccagacgccccccgcggcgccgcggctcgcgactggcgggagccgccgccgccgctgctgttggtggtggtgttggtgttactgctgccgtgtggcccgatgggcgccgaggggggcgctgtccgagccgcggccggctggggggctgcgttagacgccccgcccgtcacggggggcgcggcggtgcctctgcgtgggggggcgcggggcgtccggcggggggcgggcgggacgtagtctgctgcaagagacaacggggggcgcgatcaggttacgccccctccccggcccgccctttcctcgcccgcccgcccattcctccctcctcctcctcccccagggtccttgccgccccccgcctcaccgtcgtccaggtcgtcgtcatcctcgtccgtggtgggctccgggtgggtgggcgacagggccctcaccgtgtgcccccccagggtcaggtaccgcggggcgaaccgctgattgcccgtccagataaagtccacggccgtgcccgccctgacggcctcctcggcctccatgcgggtctgggggtcgttcacgatcgggatggtgctgaacgacccgctgggcgtcacgcccactatcaggtacaccagcttggcgttgcacagcgggcaggtgttgcgcaattgcatccaggttttcatgcacgggatgcagaagcggtgcatgcacgggaaggtgtcgcagcgcaggtggggcgcgatctcatccgtgcacacggcgcacacgtcgccctcgtcgctccccccgtcctctcgagggggggcgcccccgcaactgccggggtcttcctcgcggggggggctcccccccgagaccgcccccccatccacgccctgcggccccagcagccccgtctcgaacagttccgtgtccgtgctgtccgcctcggaggcggagtcgtcgtcatggtggtcggcgtccccccgcccccccacttcggtctccgcctcagagtcgctgctgtccggcaggtctcggtcgcagggaaacacccagacatccggggcgggctaaggggaaaaaaggggggcgggtaagaatggggggggatttcccgcgtcaatcagcgcccacgagttccccctctccccccccgcctcacaaagtcctgcccccctgctggcctcggaagaggggggagaaaggggtctgcaaccaaaggtggtctgggtccgtcctttggatcccgacccctcttcttccctcttctcccgccctccagacgcaccggagtcgggggtcccacggcgtcccccaaatatggcgggcggctcctccccacccccctagatgcgtgtgagtaaggggggcctgcgtatgagtcagtggggaccacgcccccaacacggcgaccccggtccctgtgtgtttgttgtgggggcgtgtctctgtgtatgagtcagggggtcccacggcgaccccgggccctgcgtctgagtcaaaggggccatgtgtatgtgttgggggtctgtatatataaagtcagggggtcacatggcgacccccaacagggcgaccccggtccctgtatatatagggtcagggggttccgcgccccctaacatggcgcccccggtccctgtatatatagtgtcacggggttccacgccccctaacatggcgccccaacatggcgcccggctcccgtgtatgagtgggggtcccccaacatgggggccggttccagggtaagggtcgggggtcccccaacatggcgccccccaatatggcgccccagacatggcgcccggcccctcacctcgcgctgggggcggccctcaggccggcgggtactcgctccggggcggggctccatgggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagaccgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggaccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcgggcccgccttgggggcggaccggctcccagcgtatatatgcgcggctcctgccatcgtctctccggagagcggcttggtgcggagctcccgggagctccgcggaagacccaggcgcctcgggtgtaacgttagaccgagttcgccgggccggctccgcgggccagggcccgggcacgggcctcgggccccaggcacggcccgatgaccgcctcggcctccgccacccggcgccggaaccgagcccggtcggcccgctcgcgggcccacgagccgcggcgcgccaggcgggcggccgaggcccagaccaccaggtggcgcacccggacgtggggcgagaagcgcacccgcgcgggggtcgcgggggtcgcgggggtcgcgggggtcgcgggggtcgcgggggtcgcgggggtcgcgggggtcgcggggggctccggcgccccctccccgcccgcgcgtcgcaggcgcaggcgcgccaggtgctccgcggtgacgcgcaggcggagggcgaggcgcggcggaaggcggaaggggcgcgagggggggtgggaggggtcagccccgccccccgggcccacgccgggcggtggggaccggggccggggggcggcggcggtgggccgggcctctggcgccggctcgggcggggggctgtccggccagtcgtcgtcatcgtcgtcgtcggacgcggactcgggaacgtggagccactggcgcagcagcagcgaacaagaaggcgggggcccaccggcggggggcggcggcggggcggccgcgggcgcgctcctgaccgcgggttccgagttgggcgtggaggttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccgcgctttaaagggccgcgcgcgacccccggggggtgtgtttcgggggggcccgtttttggggtctggccgctcctcccccgctcctccccgtctgtgggtggggctcctcccccgctcctcccccgctcctcccccgctcctccccgtctgtgggtggggctcctcccccgctcccgcggccccgccccccacgcccgccgcgcgcgcgcacgccgcccggaccgccgcccgccttttttgcgcgccgccccgcgcgcggggggcccgggctgccacaggtgtaacaacaccaacagaacaccaacagcacggcgcaccggcgactccggttcctcatccacacgtcacgtcatccaacacacctgcccaacaacacaactcacagcgacaactcaccgcgcaacaactcctgttcctcatccacacgtcaccgcgcaccccccgctcctccagacgtcccccagcgcaacacgccgctcctgtcacacaccacagccccagccctccccagccccagccctccccagccccagccctccccagccccagccctccccagccccagccctccccagccccagccctccccagccccagccctccccagccccagccctccccagccccagccctccccagccccagccctccccagccccagccctccccagccccagccctccccagccgcgtcccgcgctccctcgggggggttcgggcatctctacctcagtgccgccaatctcaggtcagagatccaaaccctccgggggcgcccgcgcaccaccaccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgaataaacaacgctactgcaaaacttaatcaggtcgttgccgtttattgcgtcttcgggtttcacaagcgccccgccccgtcccggcccgttacagcaccccgtccccctcgaacgcgccgccgtcgtcttcgtcccaggcgccttcccagtccacaacgtcccgtcgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcgcggtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcgcgcgccgccggccagcgcacggcgcactgcacggcgggcagcaggtcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgcgcgcccagcgccgacacgtcgggggcgccggtccagttgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttctcgtcgtcgtcgccgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacagcgccacggggcgcgggcgggcctgcgccgcggcggcccggggcgccgcgggctgggcgggggcgggctcgggccccgggggcgtggaggggggcgcgggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggccgcgacccccgaagacgaagaagagcggcgcggacccgccgccagcagggggcgcaggctctggttctcaaacagcaggtccgcggcggcggcggccgcggagctcggcaggcgcgggtcccgcggcagcgcgggacccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactccacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcggcgacgacggcggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggccgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggcttccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctctgcggcggctgggccggcgggcgcggcgacaggcggtccgtggggtccggatacgcgccgcgtagcggggcctcccgtgcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagggccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacgactgcgacggcggacgtagcacggtaggtcacctacggactctcgatggggagggggcgagacccacggaccccgacgacccccgccgtcgacgcggaactagcgcggaccggtcgatgcttgggtgggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgcccccacggcgggcccgccccgaacggaattccattatgcacgaccccgccccgacgccggcacgccgggggcccgtggccgcggcccgttggtcgaacccccggccccgcccatccgcgccatctgccatgggcggggcgctagggcgggtgggcccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggcccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagctgctgcggagcacgcggaccgggagcgggactcgcagagggccgtcggagcggacggcgtcggcatcgcgacgccccggctcgggatcgggatcgcatcggaaagggacacgcggaaagacccacccaccccacccacgaaacacaggggacgcaccccgggggcctccgacgacagaaacccaccggtccgcctttttgcacgggtaagcaccttgggtgggcggaggagggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcggggcgaccggcggcgaccgttgcgtggaccgcttcctgctcgtcggggcgaccggcggcgaccgttgcgtggaccgcttcctgctcgtcgggggggggggggggaagccactgtggtcctccgggacgttttctggatggccgacatttccccaggcgcttttgcgccttgtgtaaaagcgcggcgtcccgctctccgatccccgcccctgggcacgcgcaagcgcaagcgccctgcccgccccctctcatcggagtctgaggtcgaaaccgatacagccttggagtctgaggtcgaatccgagacagcatcggattcgaccgagtctggggaccaggaggaagccccccgcatcggtggccgtagggccccccggaggcttggggggcggttttttctggacatgtcggcggaatccaccacggggacggaaacggatacggcggtgtcggacgaccccgacgacacgtccgactggtcttatgacgacattcccccacgacccaagcgggcccgggtaaacctgcggctcacgagctctcccgatcggcgggatggggttatttttcctaagatggggcgggtccggtctacccgggaaacgcagccccgggccccaaccccgtcggccccaagcccaaatgcaatgctacggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgcttgctgcaggtgtccggcggaacctggggcatgcacctgcgcaacaccatacgggaggtggaggctcgattcgacgccaccgcggaacccgtgtgcaaacttccttgtttggaggccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgccgcagaagaccgcaagtgtctggacggctgccggaaaatgcgcttctccaccgcctgcccctatccgtgtagcgacacgtttctccggccgtgagtccggtcgccccgacccctttgtatgtcaccaaaataaaagaccaaaatcaaagcgtttgtcccagcgtcttaatggcgggaagggcggagagaaacagaccacgcggacatggggggtgtttgggggtttattggcaccgggggctaaagggtggtaaccggatagcagatgtgaggaagtcggggccgttcgccgcgaacggcgatcagagggtcagtttcttgcggaccacggcccggcgatgtgggttgctcgtctgggacctcgggcatgcccatacacgcacaacacggacgccgcaccggatgggacgtcgtaagggggcctggggtagctgggtggggtttgtgcagagcaatcagggaccgcagccagcgcatacaatcgcgctcccgtccgtttgtcccgggcagtaccacgccgtactggtattcgtaccggctgagcagggtctccagggggtggttgggggccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcacacacagatccgccgcgcctaccacccacaagtggtagatgcgaggggggctgggttggtctcggtgcagcagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccggacgaacgacgaccggggtacccaggggttctgatgccaaaatgcggcactgcctaagcaggaagctccacagggccgggcttgcgtcgacggaagtccggggcagggcgttgttctggtcaaggagggtcattacgttgacgacaacaacgcccatgttggtatattacaggcccgtgtccgatttggggcacttgcagatttgtaaggccacgcacggcggggagacaggccgacgcgggggctgctctaaaaatttaagggccctacggtccacagacccgccttcccggggggggggcccttggagcgaccggcagcgtaggcgtccgggggaggggagggtgatttacgggggggtaggtcagggggtgggtcgtcaaactgccgctccttaaaaccccggggcccgtcgttcggggtgctcgttggttggcactcacggtgcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccagcgaggccgagcgcctgtttcatctgcaggagatccttgcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgtgtggcgcatctccccccggtatacgacgacgcagccgggatgagattggggccacgggatttaccgcggaagagctggacgccatggacagggaggcggctcgagccatcagccgcggcggcaagcccccctcgaccatggccaagctggtgactggcatgggctttacgatccacggagcgctcaccccaggatcggaggggtgtgtctttgatagcagccacccagattacccccaacgggtaatcgtgaaggcggggtggtacacgagcacgagccacgaggcgcgactgctgaggcgactggaccaccccgcgatcctgcccctcctggacctgcatgtcgtctccggggtcacgtgtctggtcctccccaagtaccaggccgacctgtatacctatctgagtaggcgcctgaacccgctgggacgcccgcagatcgcagcggtctcccggcagctcctaagcgccgttgactacattcaccgccagggcattatccaccgcgacattaagaccgaaaatatttttattaacacccccgaggacatttgcctgggggactttggtgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgtgacagtcagatcacccgcatcatccgacaggcccaggtccacgttgacgagttttccccgcatccagaatcgcgcctcacctcgcgctaccgctcccgcgcggccgggaacaatcgcccgccttacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgcccccggggggcggtgctgtttgcgggttggcacaaaaagaccccgacccgcgtctgtggtgtttttggcatcatgtcgccgggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtatcggactggaggaggaggaggaagaggaggagggggccggggatggcgaacatcttaaggggggagatgggacccgtgacaccctaccccagtccccgggtccagccgtcccgttggccggggatgacgagaaggacaaacccaaccgtcccgtagtcccaccccccggtcccaacaactcccccgcgcgccccgagaccagtcgaccgaagacaccccccaccagtatcgggccgctggcaactcgacccacgacccaactcccctcaaaggggcgacccttggttccgacgcctcaacataccccgctgttctcgttcctcactgcctcccccgccctggacaccctcttcgtcgtcagcaccgtcatccacaccttatcgtttttgtgtattggtgcgatggcgacacacctgtgtggcggttggtccagacgcgggcgacgcacacaccctagcgtgcgttacgtgtgcctgccgtccgaacgcgggtagggtatggggcgggggatggggagagcccacatgcggaaagcaagaacaataaaggcggtggtatctagttgatatgcatctctgggtgtttttggggtgtggcggacgcggggcggtcattggacggggtgcagttaaatacatgcccgggacccatgaagcatgcgcgacttccgggcctcggaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcgtgtctgcttgagctcctgcgtcgtacgtgccgcgggtgggggcgttaccatccctacatggacccagttgtcgtataattcccccccccccttctccgcatgggtgatgtcgggtccaaactcccgacaccaccagctggcatggtataaatcaccggtgcgccccccaaaccatgtccggcagggggatgggggggcgaatgcggagggcacccaacaacaccgggctaaccaggaaatccgtggccccggcccccaataaagatcgcggtagcccggccgtgtgacactatcgtccataccgaccacaccgacgaatcccctaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccgcactcaaaaagccccaaagcgcatacgcctcccccacatccgggaagacgaccagccgtcctcgcaccagcccttgttttactagatacccccccttaatgggtgcgggggggtcaggtctgcggggttgggatgggaccttaactccatataaagcgagtctggaaggggggaaaggcggacagtcgataagtcggtagcgggggacgcgcacctgttccgcctgtcgcacccacagctttttttgcgaaccgtcccgttccgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatggggtcgtagagctgtggcactaccccatgggacacaaatgcccacgggtcgtgcatgtcgtcacggtgaccgcgtgcccacgtcgccccgccgtggctttcgccctgtgtcgcgcgaccgacaacactcacagccccgcatatcccaccctggagctgaatctggcccaacagccgcttttgcgggtccggagggcgacgcgtgactatgccggggtgtacgtgttacgcgtatgggtcgtggacgcaccaaacgccagcctgtttgtcctggggatggccatagccgccgaagggactctggcgtacaacggctcggcccatggctcctgcgacccgaaactgcttccgtattcggccccgcgtctggccccggcgagcgtataccaacccgcccctaacccggcctccaccccctcgaccaccacctccaccccctcgaccaccacctccaccccctcgaccaccacctccaccccctcgaccaccacctccaccccctcgaccaccacctccaccccctcgaccaccacctccaccccctcgaccaccatccccgctccccaagcatcgaccacacccttccccacgggagacccaaaaccccaacctcacggggtcaaccacgaacccccatcgaatgccacgcgagcgacccgcgactcgcgatacgcgctaacggtgacccagataatccagatagccatccccgcgtccattatagccctggtgtttctggggagctgtatttgctttatacacagatgtcaacgccgctaccgacgctcccgccgcccgatttacaacccccagatacccacgggcatctcatgcgcggtgaacgaagcggccatggcccgcctcggagccgagctcaaatcgcatccgagcaccccccccaaatcccggcgccggtcgtcacgcacgccaatgccctccctgacggccatcgccgaagagtcggagcccgcgggggcggctgggcttccgacgccccccgtggaccccacgacatccaccccaacgcctcccctgttggtataggtccacggccactggccgggggcaccacataaccgaccgcagtcactgagttgggaataaaccggtattatttacctatatccgtgtatgtccatttctttcccccccccccccccccggaaacccaaagaaggaagcaaagaatggatgggaggagttcaggaagccggggagagggcccgcggcgcatttaaggcgttgttgtgttgactttggctcttctggcgggttggtgcggtgctgtttgttgggctcccattttacccgaagatcggctgctatccccgggacatggatcgcggggcggtggtggggtttcttctcggtgtttgtgttgtatcgtgcttggcgggaatgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacgaacggacttcgtgtggcaggagcgcgcggccgtggttaaccggagtctggttattcacggggtccgagagacggacagcggcctgtataccctgtccgtgggcgacataaaggacccggctcgccaagtggcctcggtggtcctggtggtgcaaccggccccagttccgaccccacccccgaccccagccgattacgacgaggatgacaatgacgagggcgagggcgaggacgaaagtctagccggcactcccgccagcgggaccccccggctcccgcctccccccgcccccccgaggtcttggcccagcgcccccgaagtctcacacgtgcgtggggtgaccgtgcgtatggagactccggaagctatcctgttttcccccggggaggcgtttagcacgaacgtctccatccatgccatcgcccacgacgaccagacctacaccatggacgtcgtctggttgaggttcgacgtgccgacctcgtgtgccgagatgcgaatatacgaatcgtgtctgtatcacccgcagctcccagagtgtctgtccccggccgacgctccgtgcgccgcgagtacgtggacgtctcgcctggccgtccgcagctacgcggggtgttccagaacaaaccccccgccgcgctgttcggccgaggctcacatggagcccttcccggggctggcgtggcaggcggcctccgtcaatctggagttccgggacgcgtccccacaacactccggcctgtatctgtgcgtggtgtacgtcaacgaccatattcacgcatggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcgctggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcttgtgtgggattgcgtggtatgtgacgtcaattgcccgaggcgcataaagggccggtggtccgcctagccgcagcaaattaaaaatcgtgagtcactgcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgtcgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttccccccatcaccccgcaatttgcacccagcctttaactacattaaattgggttcgattggcaatgttgtctcccggttgatttttgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtggcaaggaagaaacaagcccgaccaccagacagaaaatgtaaccatacccaaaccgactctgggggctgtttgtggggtcggaaccataggatgaacaaaccaccccgtacctcccgcacccttgggtgcgggtggctcatcggcatctgtccggtatgggttgttccccacccactcgcgttcggacgtcttagaatcatggcggtttctatgccgacatcggtttctcccccgcaataagacacgatgcgataaaatctgtttgtgaaatttattaagggtacaaattgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatgtgtcggtgggtttggacgcggggggccatggtgggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcggaagaaggcccggtggtccgcgggcaacatcaccaggtgcgcaagcgcccgggcgtccagagggtagagccctgagtcatccgaggttggctcatcgcccgggtcatgccgcaagtgcgtgtgggttgggcttccggtgggcgggacgcgaaccgcggtgtggagcccgacgcgggcccgagcgtacgctccatcttgtggggagaaggggtctgggctcgccaggggggcatacttgcccgggctatacagacccgcgagccgtacgtggttcgcggggggtgcgtggggtccggggctcccggggaggccggggctcccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccctgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagccacggggggaaccaccctccgcccagaaacttgggcgatggtcgtacccgggactcaacgggttaccggattacggggactgtcggtcacggtcccgccggttcttcgatgtgccacacccaaggatgcgttgggggcgattttgggcagcagcccgggagagcgcagcagaggacgctccgggtcgtgcacggcggttctggccgcctcccggtcctcacgcccccttttattgatctcatcgcgtacgtcggcgtacgtcctgggcccaacccgcatgttgtccaggaaggtgtccgccatttccagggcccacgacatgctcccccccccccccccgacgagcaggaagcggtccacgcaacggtcgccgccggtcgccccgacgagcaggaagcggtccacgcaacggtcgccgccggtcgccccgacgagcaggaagcggtccacgcaacggtcgccgccggtcgcctcgacgaggacgttcctcctgcgggaaggcacgaacgcgggtgagccccctcctccgcccccgcgtcccccctcctccgcccccgcgtcccccctcctccgcccccgcgtcccccctcctccgcccccgcgtcccccctcctccgccctcctccgcccacccaaggtgcttacccgtgcaaaaaggcggaccggtgggtttctgtcgtcggaggcccccggggtgcgtcccctgtgtttcgtgggtggggtgggtgggtctttccgcgtgtccctttccgatgcgatcccgatcccgagccggggcgtcgcgatgccgacgccgtccgctccgacggccctctgcgagtcccgctcccggtccgcgtgctccgcagcagctcccgtcgttcgtggccggcgccgtctgcgggcgtcggtcgcgccgggcctttatgtgcgccggagagacccgccccccgccgcccgggcccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccggcgtccggcgggcgggaccgggggcccggggacggccaacgggcgcgcggggctcgtatctcattaccgccgaaccgggaagtcggggcccgggccccgccccctgcccgttcctcgttagcatgcggaacggaagcggaaaccgccggatcgggcggtaatgagatgccatgcggggcggggcgcgggcccacccgccctagcgccccgcccatggcagatggcgcggatgggcggggccgggggttcgaccaacgggccgcggccacgggcccccggcgtgccggcgtcggggcggggtcgtgcataatggaattccgttcggggcgggcccgccgtgggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccatcgagagtccgtaggtgacctaccgtgctacgtccgccgtcgcagtcgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggccctgctggcctccatggtagaggaggccgtccggacgatcccgacgcccgaccccgcggcctcgccgccccggacccccgcctttcgagccgacgacgatgacggggacgagtacgacgacgcagccgacgccgccggcgaccgggccccggcccggggccgcgcacgggaggccccgctacgcggcgcgtatccggaccccacggaccgcctgtcgccgcgcccgccggcccagccgccgcagagacgtcgtcacggccggcggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggaagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcggcctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgccgtcgtcgccgccgccgccgcaccgggcgagcgcgcggtgcccgccgggtacggcgccgcggggatcctcgccgccctggggcggctgtccgccgcgcccgcctcccccgcggggggcgacgaccccgacgccgcccgccacgccgacgccgacgacgacgccgggcgccgcgcccaggccggccgcgtggccgtggagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggtcccgcgctgccgcgggacccgcgcctgccgagctccgcggccgccgccgccgcggacctgctgtttgagaaccagagcctgcgccccctgctggcggcgggtccgcgccgctcttcttcgtcttcgggggtcgcggccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccgcgcccccctccacgcccccggggcccgagcccgcccccgcccagcccgcggcgccccgggccgccgcggcgcaggcccgcccgcgccccgtggcgctgtcgcgccggcccgccgagggccccgaccccctgggcggctggcggcggcagcccccggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcggcgacgacgacgagaacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaactggaccggcgcccccgacgtgtcggcgctgggcgcgcagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgacctgctgcccgccgtgcagtgcgccgtgcgctggccggcggcgcgcgacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcaccgcgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacggcccgggggccctgccgccggcgccgcccgggattcgctgggcctcggccacgggccgcagcggcaccgtgctggcggcggcgggggccgtggaggtgctgggggcggaggcgggcttggccacgcccccgcgacgggacgttgtggactgggaaggcgcctgggacgaagacgacggcggcgcgttcgagggggacggggtgctgtaacgggccgggacggggcggggcgcttgtgaaacccgaagacgcaataaacggcaacgacctgattaagttttgcagtagcgttgtttattcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcggtggtggtgcgcgggcgcccccggagggtttggatctctgacctgagattggcggcactgaggtagagatgcccgaacccccccgagggagcgcgggacgcggctggggagggctggggctggggagggctggggctggggagggctggggctggggagggctggggctggggagggctggggctggggagggctggggctggggagggctggggctggggagggctggggctggggagggctggggctggggagggctggggctggggagggctggggctggggagggctggggctgtggtgtgtgacaggagcggcgtgttgcgctgggggacgtctggaggagcggggggtgcgcggtgacgtgtggatgaggaacaggagttgttgcgcggtgagttgtcgctgtgagttgtgttgttgggcaggtgtgttggatgacgtgacgtgtggatgaggaaccggagtcgccggtgcgccgtgctgttggtgttctgttggtgttgttacacctgtggcagcccgggccccccgcgcgcggggcggcgcgcaaaaaaggcgggcggcggtccgggcggcgtgcgcgcgcgcggcgggcgtggggggcggggccgcgggagcgggggaggagccccacccacagacggggaggagcgggggaggagcgggggaggagcgggggaggagccccacccacagacggggaggagcgggggaggagcggccagaccccaaaaacgggcccccccgaaacacaccccccgggggtcgcgcgcggccctttaaagcgcggcggcgggcagcccgggccccccgcgg

SEQ ID NO: 2 - protein, 1300 aa, unidentified
  source 1..1300: note = human herpesvirus 1 strain McKrae ICP4
MASENKQRPGSPGPTDGPPPTPSPDRDERGALGWGAETEEGGDDPDHDPDHPHDLDDARRDGRAPAAGTDAGEDAGDAVSPRQLALLASMVEEAVRTIPTPDPAASPPRTPAFRADDDDGDEYDDAADAAGDRAPARGRAREAPLRGAYPDPTDRLSPRPPAQPPQRRRHGRRRPSASSTSSDSGSSSSSSASSSSSSSDEDEDDDGNDAADHAREARAVGRGPSSAAPEAPGRTPPPPGPPPLSEAAPKPRAAARTPAASAGRIERRRARAAVAGRDATGRFTAGQPRRVELDADAASGAFYARYRDGYVSGEPWPGAGPPPPGRVLYGGLGDSRPGLWGAPEAEEARRRFEASGAPAAVWAPELGDAAQQYALITRLLYTPDAEAMGWLQNPRVVPGDVALDQACFRISGAARNSSSFITGSVARAVPHLGYAMAAGRFGWGLAHAAAAVAMSRRYDRAQKGFLLTSLRRAYAPLLARENAALTGAAGSPGAGADDEGVAAAVVAAAAAPGERAVPAGYGAAGILAALGRLSAAPASPAGGDDPDAARHADADDDAGRRAQAGRVAVECLAACRGILEALAEGFDGDLAAVPGLAGARPASPPRPEGPAGPASPPPPHADAPRLRAWLRELRFVRDALVLMRLRGDLRVAGGSEAAVAAVRAVSLVAGALGPALPRDPRLPSSAAAAAADLLFENQSLRPLLAAGPRRSSSSSGVAAAASAAPREGRKRKSPGPARPPGGGGPRPPKTKKSGADAPGSDARAPLPAPAPPSTPPGPEPAPAQPAAPRAAAAQARPRPVALSRRPAEGPDPLGGWRRQPPGPSHTAAPAAAALEAYCSPRAVAELTDHPLFPVPWRPALMFDPRALASIAARCAGPAPAAQAACGGGDDDENPHPHGAAGGRLFGPLRASGPLRRMAAWMRQIPDPEDVRVVVLYSPLPGEDLAGGGASGGPPEWSAERGGLSCLLAALANRLCGPDTAAWAGNWTGAPDVSALGAQGVLLLSTRDLAFAGAVEFLGLLASAGDRRLIVVNTVRACDWPADGPAVSRQHAYLACDLLPAVQCAVRWPAARDLRRTVLASGRVFGPGVFARVEAAHARLYPDAPPLRLCRGGNVRYRVRTRFGPDTPVPMSPREYRRAVLPALDGRAAASGTTDAMAPGAPDFCEEEAHSHRACARWGLGAPLRPVYVALGREAVRAGPARWRGPRRDFCARALLEPDDDAPPLVLRGDDDGPGALPPAPPGIRWASATGRSGTVLAAAGAVEVLGAEAGLATPPRRDVVDWEGAWDEDDGGAFEGDGVL

SEQ ID NO: 3 - protein, 420 aa, unidentified
  source 1..420: note = human herpesvirus 1 strain McKrae ICP22
MADISPGAFAPCVKARRPALRSPPLGTRKRKRPARPLSSESEVETDTALESEVESETASDSTESGDQEEAPRIGGRRAPRRLGGRFFLDMSAESTTGTETDTAVSDDPDDTSDWSYDDIPPRPKRARVNLRLTSSPDRRDGVIFPKMGRVRSTRETQPRAPTPSAPSPNAMLRRSVRQAQRRSSARWTPDLGYMRQCINQLFRVLRVARDPHGSANRLRHLIRDCYLMGYCRARLAPRTWCRLLQVSGGTWGMHLRNTIREVEARFDATAEPVCKLPCLEARRYGPECDLSNLEIHLSATSDDEISDATDLEAAGSDHTLASQSDTEDAPSPVTLETPEPRGSLAVRLEDEFGEFDWTPQEGSQPWLSAVVADTSSVERPGPSDSGAGRAAEDRKCLDGCRKMRFSTACPYPCSDTFLRP

SEQ ID NO: 4 - protein, 88 aa, unidentified
  source 1..88: note = human herpesvirus 1 strain McKrae ICP47
MSWALEMADTFLDNMRVGPRTYADVRDEINKRGREDREAARTAVHDPERPLLRSPGLLPKIAPNASLGVAHRRTGGTVTDSPRNPVTR

SEQ ID NO: 5 - DNA, 4020 bp, unidentified
  source 1..4020: mol_type = genomic DNA; note = human herpesvirus 1 strain McKrae ICP4
tttattgcgtcttcgggtttcacaagcgccccgccccgtcccggcccgttacagcaccccgtccccctcgaacgcgccgccgtcgtcttcgtcccaggcgccttcccagtccacaacgtcccgtcgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcgcggtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcgcgcgccgccggccagcgcacggcgcactgcacggcgggcagcaggtcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgcgcgcccagcgccgacacgtcgggggcgccggtccagttgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttctcgtcgtcgtcgccgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacagcgccacggggcgcgggcgggcctgcgccgcggcggcccggggcgccgcgggctgggcgggggcgggctcgggccccgggggcgtggaggggggcgcgggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggccgcgacccccgaagacgaagaagagcggcgcggacccgccgccagcagggggcgcaggctctggttctcaaacagcaggtccgcggcggcggcggccgcggagctcggcaggcgcgggtcccgcggcagcgcgggacccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactccacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcggcgacgacggcggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggccgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggcttccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctctgcggcggctgggccggcgggcgcggcgacaggcggtccgtggggtccggatacgcgccgcgtagcggggcctcccgtgcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagggccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacgactgcgacggcggacgtagcacggtaggtcacctacgg

SEQ ID NO: 6 - DNA, 1320 bp, unidentified
  source 1..1320: mol_type = genomic DNA; note = human herpesvirus 1 strain McKrae ICP22
ggtcctccgggacgttttctggatggccgacatttccccaggcgcttttgtgccttgtgtaaaagcgcggcgtcccgctctccgatccccgcccctgggcacgcgcaagcgcaagcgccctgcccgccccctctcatcggagtctgaggtcgaatccgagacagccttggagtctgaggtcgaatccgagacagcatcggattcgaccgagtctggggaccaggaggaagccccccgcatcggtggccgtagggccccccggaggcttggggggcggttttttctggacatgtcggcggaatccaccacggggacggaaacggatgcgtcggtgtcggacgaccccgacgacacgtccgactggtcttgtgacgacattcccccacgacccaagcgggcccgggtaaacctgcggctcactagctctcccgatcggcgggatggggttatttttcctaagatggggcgggtccggtctacccgggaaacgcagccccgggcccccaccccgtcggccccaagcccaaatgcaatgctccggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgcttgctgcaggtgtccggcggaacctggggcatgcacctgcgcaacaccatacgggaggtggaggctcgattcgacgccaccgcagaacccgtgtgcaagcttccttgtttggaggccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgcagcagaagaccgcaagtgtctggacggctgccggaaaatgcgcttctccaccgcctgcccctatccgtgcagcgacacgtttctccggccgtgagtccggtcgccccgacccccttgtatgtccccaaa

SEQ ID NO: 7 - DNA, 360 bp, unidentified
  source 1..360: mol_type = genomic DNA; note = human herpesvirus 1 strain McKrae ICP47
tccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatgttgtccaggaaggtgtccgccatttccagggcccacgacatgcttttccccccgacgagcaggaagcggtccacgcaacggtcgccgccggtcgcct

SEQ ID NO: 8 - DNA, 523 bp, Human cytomegalovirus
  source 1..523: mol_type = genomic DNA
gaagatctttggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaat

SEQ ID NO: 9 - DNA, 1199 bp, Homo sapiens
  source 1..1199: mol_type = genomic DNA
aatgggtttgggtgtgtgtaaatgagtgtgaccggaagcgagtgtgagcttgatctaggcagggaccacacagcactgtcacacctgcctgctctttagtagaggactgaagtgcgggggtgggggtacggggccggaatagaatgtctctgggacatcttggcaaacagcagccggaagcaaaggggcagctgtgcaaacggctcaggcaggtgatggatggcagggtaggaagggggaggtccagaggtctggatggaggcttccgcatctgtaccttgcaactcacccctcaggcccagcaggtcatcggccccctcctcacacatgtaatgacgtagaagagtaccccgggacagtccggggagatggagattcggaaagtatccatggagctcttacagaatcccctgtgcggaccaggaaactcttgtagatccctgcctatctgaggcccaggcgctgggctgtttctcacaatattccttcaagatgagattgtggtccccatttcaaagatgagtacactgagcctctgtgaagttacttgcccatgatcacacaaccaggaattgggccaactgtaattgaactcctgtctaacaaagttcttgctcccagctccgtctcttgtttcccacgagccctggccctctgtgggtaataccagctactggagtcagatttcttgggcccagaacccacccttaggggcattaacctttaaaatctcacttgggcaggggtctgggatcagagttggaagagtccctacaatcctggaccctttccgccaaatcgtgaaaccaggggtggagtggggcgagggttcaaaaccaggccggactgagaggtgaaattcaccatgacgtcaaactgccctcaaattcccgctcactttaagggcgttacttgttggtgcccccaccatcccccaccatttccatcaatgacctcaatgcaaatacaagtgggacggtcctgctgacgcctccaggttctggaagcatgagggtgacgcacccaggggcaaaggacccctccgcccattggttgctgtgcactggcggaactttcccgacccacagcggcgggaataagagcagtcgctggcgctgggaggcatcagagacactgcccagcccaagtgtcgccgccgcttccacagggctctggctggacgccgccgccgccgctgc

SEQ ID NO: 10 - DNA, 225 bp, synthetic construct
  misc_feature 1..225: note = bovine growth hormone polyadenylation signal
  source 1..225: mol_type = other DNA
ggatcccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagatcttc

SEQ ID NO: 11 - protein, 7 aa, unidentified
  source 1..7: note = human herpesvirus 1 strain McKrae
STPSTTT

SEQ ID NO: 12 - DNA, 16 bp, unidentified
  source 1..16: mol_type = genomic DNA; note = human herpesvirus 1 strain McKrae
gcacccccactcccac

SEQ ID NO: 13 - DNA, 16 bp, unidentified
  source 1..16: mol_type = genomic DNA; note = human herpesvirus 1 strain McKrae
ccccagccctccccag

SEQ ID NO: 14 - DNA, 17 bp, unidentified
  source 1..17: mol_type = genomic DNA; note = human herpesvirus 1 strain McKrae
cccctcgccccctcccg

SEQ ID NO: 15 - protein, 14 aa, unidentified
  source 1..14: note = human herpesvirus 1 strain KOS
AASAPDAADALAAA

SEQ ID NO: 16 - protein, 13 aa, unidentified
  source 1..13: note = human herpesvirus 1 strain McKrae
GPRRSSSSSGVAA

SEQ ID NO: 17-18 - skipped (2 entries)

SEQ ID NO: 19 - protein, 9 aa, unidentified
  source 1..9: note = human herpesvirus 1
TAATGARAT